(19) United States
(12) Patent Application Publication
(10) Pub. No.: US 2022/0348958 A1
(43) Pub. Date: Nov. 3, 2022

(54) Enhancing Production of Lentiviral Vectors

(71) Applicant: Oxford BioMedica (UK) Limited, Oxford (GB)

(72) Inventors: Daniel Farley, Oxford (GB); Jordan Wright, Oxford (GB)

(21) Appl. No.: 17/629,378

(22) PCT Filed: Jul. 23, 2020
(86) PCT No.: PCT/GB2020/051760

§ 371 Date: Jan. 22, 2022

(30) Foreign Application Priority Data
Jul. 23, 2019 (GB) 1910518.8
Feb. 13, 2020 (GB) 2001997.2

(51) Int. Cl. C12N 15/86; C12N 15/113

(52) U.S. Cl. CPC C12N 15/86; C12N 15/1132; C12N 2310/14; C12N 2310/531; C12N 2740/15022; C12N 2740/15062; C12N 2740/15043; C12N 2740/15052; C12N 2740/16043; C12N 2740/16052

(57) ABSTRACT

A modified U1 snRNA, wherein said modified U1 snRNA has been modified to bind to a nucleotide sequence within the packaging region of a lentiviral vector genome sequence.

### CROSS-REFERENCE TO RELATED APPLICATIONS

This application is the national stage under 35 U.S.C. 371 of International Application No. PCT/GB2020/051760, filed on Jul. 23, 2020, and claiming priority to GB Application No. 1910518.8, filed on Jul. 23, 2019 and to GB Application No. 2001997.2, filed on Feb. 13, 2020.

### FIELD OF THE INVENTION

The invention relates to the production of lentiviral vectors in eukaryotic cells. More specifically, the present invention relates to the design and co-expression of modified U1 snRNA during production of lentiviral vectors in order to increase output titres.

### BACKGROUND TO THE INVENTION

The development and manufacture of viral vectors towards vaccines and human gene therapy over the last several decades is well documented in scientific journals and in patents. The use of engineered viruses to deliver transgenes for therapeutic effect is wide-ranging. Contemporary gene therapy vectors based on RNA viruses such as γ-retroviruses and lentiviruses (Muhlebach, M. D. et al., 2010, Retroviruses: Molecular Biology, Genomics and Pathogenesis, 13:347-370; Antoniou, M. N., Skipper, K. A. & Anakok, O., 2013, Hum. Gene Ther., 24:363-374), and DNA viruses such as adenovirus (Capasso, C. et al., 2014, Viruses, 6:832-855) and adeno-associated virus (AAV) (Kotterman, M. A. & Schaffer, D. V., 2014, Nat. Rev. Genet., 15:445-451) have shown promise in a growing number of human disease indications. These include ex vivo modification of patient cells for hematological conditions (Morgan, R. A. & Kakarla, S., 2014, Cancer J., 20:145-150; Touzot, F. et al., 2014, Expert Opin. Biol. Ther., 14:789-798), and in vivo treatment of ophthalmic (Balaggan, K. S. & Ali, R. R., 2012, Gene Ther., 19:145-153), cardiovascular (Katz, M. G. et al., 2013, Hum. Gene Ther., 24:914-927), neurodegenerative diseases (Coune, P. G., Schneider, B. L. & Aebischer, P., 2012, Cold Spring Harb. Perspect. Med., 4:a009431) and tumor therapy (Pazarentzos, E. & Mazarakis, N. D., 2014, Adv. Exp. Med Biol., 818:255-280). As the successes of these approaches in clinical trials begin to build towards regulatory approval and commercialisation, attention has focused on the emerging bottleneck in mass production of good manufacturing practice (GMP) grade vector material (Van der Loo J C M, Wright J F., 2016, Human Molecular Genetics, 25(R1):R42-R52).

A way to overcome this challenge is to find new ways to maximise titre during viral vector production. Thus, there is a need in the art to provide alternative methods of producing viral vectors which help to address the known issues associated with the mass production of GMP grade vector material.

Common methods of viral vector manufacture include the transfection of primary cells or mammalian/insect cell lines with vector DNA components, followed by a limited incubation period and then harvest of crude vector from culture media and/or cells (Merten, O-W., Schweizer, M., Chahal, P., & Kamen, A. A., 2014, Pharmaceutical Bioprocessing, 2:183-203). In other cases, producer cell lines (PrCLs; where all of the necessary vector component expression cassettes are stably integrated into the production cell DNA) are used during transfection-independent approaches, which is advantageous at larger scales. The efficiency of lentiviral vector manufacturing is typically affected by several factors at the ‘upstream phase’, including [1] viral serotype/pseudotype employed, [2] transgenic sequence composition and size, [3] media composition/gassing/pH, [4] transfection reagent/process, [5] chemical induction and vector harvest timings, [6] cell fragility/viability, [7] bioreactor shear-forces and [8] impurities. Clearly there are other factors to consider during the ‘downstream’ purification/concentration phase (Merten, O-W. et al., 2014, Pharmaceutical Bioprocessing, 2:237-251).

One important aspect of optimisation is the relative abundance of lentiviral vector components—GagPol, envelope, rev and vector genome RNA (vRNA)—during the upstream phase of production. For approaches requiring transient transfection of plasmid DNA encoding these components, the optimal ‘mass’ ratio of these plasmids is usually identified during optimisation. The optimal ratio is also effectively solved by screening many PrCLs containing all of the components stably integrated into the host DNA; those PrCLs with greatest output being the clones that harbour the expression cassettes at loci that result in expression of each individual component at or close to the optimal component ratio. The present inventors note that often the genomic vRNA component can be the limiting factor in PrCLs; this is supported by the report that high-titre output by PrCL clones correlates with high copy number of the vector genome cassette (Sheridan, P. L. et al., 2000, Mol. Ther, 2(3):262-275). Typically the largest proportion of plasmid DNA in the optimal ratio of component plasmids during transient transfection approaches, even when size of plasmid is taken into consideration. This indicates that the production of genome vRNA during lentiviral vector manufacture is a key factor in generating an efficient lentiviral vector manufacturing approach.

Splicing and polyadenylation are key processes for mRNA maturation, particularly in higher eukaryotes where most protein-coding transcripts contain multiple introns. The elements within an mRNA that are required for splicing include the 5′ splice donor signal, the sequence surrounding the branch point and the 3′ splice acceptor signal. Interacting with these three elements is the spliceosome, which is formed by five small nuclear RNAs (snRNAs), including U1 snRNA, and associated nuclear proteins (snRNP). U1 snRNA is expressed by a polymerase II promoter and is present in most eukaryotic cells (Lund et al., 1984, J. Biol. Chem., 259:2013-2021). Human U1 snRNA (small nuclear RNA) is 164 nt long with a well-defined structure consisting of four stem-loops (West, S., 2012, Biochemical Society Transactions, 40:846-849). U1 snRNA contains a short sequence at its 5′-end that is broadly complementary to the 5′ splice donor sites at exon-intron junctions. U1 snRNA participates in splice-site selection and spliceosome assembly by base pairing to the 5′ splice donor site. One known function for U1 snRNA outside splicing is in the regulation of 3′-end mRNA processing: it suppresses premature polyadenylation (polyA) at early polyA signals (particularly within introns).

The repression of polyA sites within intronic sequences in cellular genes by recruitment of endogenous U1 snRNA has been well characterised (Kaida, D. et al., 2010, Nature, 468:664-8). Others have also used modified U1 snRNAs as research tools to dissect the mechanism of suppression of premature polyadenylation at the 5′LTR during HIV-1 replication (Ashe, M. P., Pearson, L. H. & Proudfoot N.J., 1997, EMBO J., 16:5752-63; Ashe, M. P., Furger, A. & Proudfoot, N.J., 2000, RNA, 6:170-7; Furger, A., Monks, J. & Proudfoot, N.J., 2001, J. Virol., 75:11735-46).

HIV-1 encodes an identical polyA signal within the R region of both 5′ and 3′LTRs; the 3′LTR polyA is active, and utilised to terminate all pre-mRNA transcription events. In order to avoid pre-mature termination at the polyA site in the 5′LTR (i.e. immediately after transcription initiation), the binding of endogenous U1 snRNA to the major splice donor (˜200 bases downstream) suppresses the activity of this polyA site, thus allowing transcription to continue to the end of the provirus cassette. It was shown that mutation of the major splice donor activated the polyA site in the 5′LTR but that suppression could be re-instated if a modified U1 snRNA (targeting a sequence adjacent to the major splice donor) was co-expressed. However, suppression of the polyA site was dependent on the close proximity of the U1 snRNA to the polyA site, and much of the work carried out was performed in artificial ‘mini-gene’ expression cassette that lack many other RNA sequences found in wild type HIV-1 or HIV-1 based lentiviral vectors.

The manipulation and use of U1 snRNAs is known in the art as ‘U1 interference’ or ‘U1i’, and has been used to develop an approach to suppress gene expression (Beckley, S. A. et al., 2001, Mol. Cell Biol., 21:2815-25; Fortes, P. et al., 2003, Proc. Nat. Acad. Sci. U.S.A., 100:8264-9). The mechanism by which U1i functions is by inhibiting the correct position and processing of polyadenylation of pre-mRNA, such that unstable mRNAs are produced, leading to reduced protein levels. Inhibition of polyadenylation requires localization of the modified U1 snRNP particle to the 3′-terminal exon of the target transcript. The nucleic acid sequence of a U1 snRNA gene is modified so that the U1 snRNA binds to a selected sequence instead of the 5′ splice donor site sequence that it uses to initiate splicing of the target gene. Inhibition of polyadenylation is accomplished by base pairing the 5′ end of the modified U1 snRNA in its associated RNP complex to the selected complementary region in the target pre-mRNA. It has been used as an antiviral approach (Bláquez, L. & Fortes, P., 2015, Adv. Exp. Med. Biol., 848:51-69), including HIV-1 (Sajic, R. et al., 2007, Nucleic Acids Res., 35:247-55; Knoepfel, S. A. et al., 2012, Antiviral Res., 94:208-16).

Others have shown that the binding of endogenous U1 snRNAs or modified U1 snRNAs to consensus or non-consensus 5′ splice sites in HIV-1 can alter viral RNA stability (Lützelberger, M. et al., 2006, J. Biol. Chem., 281:18644-51). However, the report suggests that this effect is specific to a short, novel intron found within the pol region of HIV-1; such a sequence is completely absent from the vector genome sequence based on HIV-1 lentiviral vectors.

The manipulation and use of U1 snRNAs to increase lentiviral vector titre during lentiviral vector production was unknown at the filing date of the present application.

### SUMMARY OF THE INVENTION

The present inventors have surprisingly found that the output titres of lentiviral vectors can be enhanced by co-expressing non-coding RNAs based on U1 snRNAs, which have been modified so that they no longer target the endogenous sequence (a splice donor site) but now target a sequence within the vRNA molecule. The present invention relates to such modified U1 snRNAs and a novel method to increase the production titres of lentiviral vectors. The approach consists of the co-expression of modified U1 snRNAs together with the other vector components during vector production. The modified U1 snRNAs are designed such that binding to the consensus splice donor site has been ablated by replacing it with a heterologous sequence that is complementary to a target sequence within the vector genome vRNA. The invention describes various modes of application and optimal characteristics of the modified U1 snRNAs, including target sequence and complementarity length, design and modes of expression.

In one aspect the invention provides a modified U1 snRNA, wherein said modified U1 snRNA has been modified to bind to a nucleotide sequence within the packaging region of a lentiviral vector genome sequence.

In one aspect the invention provides a modified U1 snRNA, wherein said modified U1 snRNA has been modified to be complimentary to a nucleotide sequence within the packaging region of a lentiviral vector genome sequence.

In some embodiments, the modified U1 snRNA is modified to introduce a heterologous sequence that is complementary to said nucleotide sequence.

In some embodiments, the modified U1 snRNA is modified at the 5′ end to introduce within the nine nucleotides at positions 3-to-11 said heterologous sequence.

In some embodiments, the modified U1 snRNA is modified at the 5′ end to introduce within the native splice donor annealing sequence said heterologous sequence.

In some embodiments, 1-9 nucleic acids of said native splice donor annealing sequence are replaced with said heterologous sequence. In one aspect nucleotides 1-11, encompassing the native splice donor annealing sequence, will be replaced with a heterologous sequence that is complementary to said nucleotide sequence.

In some embodiments, the modified U1 snRNA is modified at the 5′ end to replace a sequence encompassing the native splice donor annealing sequence with a heterologous sequence that is complementary to said nucleotide sequence.

In some embodiments, the heterologous sequence comprises at least 9 nucleotides of complementarity to said nucleotide sequence.

In some embodiments, the heterologous sequence comprises 15 nucleotides of complementarity to said nucleotide sequence.

In some embodiments, the packaging region of a lentiviral vector genome sequence is the beginning of the 5′ U5-domain to the terminus of the sequence derived from gag gene.

In some embodiments, the nucleotide sequence is located within the 5′ U5 domain, PBS element, SL1 element, SL2 element, SL3ψ element, SL4 element and/or the sequence derived from gag gene.

In some embodiments, the nucleotide sequence is located within the SL1, SL2 and/or SL3ψ element(s).

In some embodiments, the nucleotide sequence is located within the SL1 and/or SL2 element(s).

In some embodiments, the nucleotide sequence is located within the SL1 element.

In some embodiments, the modified U1 snRNA is a modified U1A snRNA or a modified U1A snRNA variant.

In some embodiments, the first two nucleotides at the 5′ end of the modified U1 snRNA are not AU.

In some embodiments, said lentiviral vector is derived from HIV-1, HIV-2, SIV, FIV, BIV, EIAV, CAEV or Visna lentivirus.

In some embodiments, said lentiviral vector is derived from HIV-1, HIV-2 or EIAV.

In some embodiments, said lentiviral vector is derived from HIV-1.

In some embodiments, said lentiviral vector is derived from SIV.

In a further aspect, the invention provides an expression cassette comprising a nucleotide sequence encoding a modified U1 snRNA of the invention.

In a further aspect, the invention provides a cell for producing lentiviral vectors comprising nucleotide sequences encoding vector components including gag, env, rev and the RNA genome of the lentiviral vector and at least one nucleotide sequence encoding a modified U1 snRNA of the invention.

In a further aspect, the invention provides a cell comprising a modified U1 snRNA according to the invention.

In some embodiments, the cell further comprises a nucleotide sequence encoding the RNA genome of the lentiviral vector.

In some embodiments, the cell further comprises a nucleotide sequence encoding a nucleotide of interest.

In some embodiments, said nucleotide of interest gives rise to a therapeutic effect.

In some embodiments, said nucleotide of interest encodes an enzyme, co-factor, cytokine, chemokine, hormone, antibody, anti-oxidant molecule, engineered immunoglobulin-like molecule, single chain antibody, fusion protein, immune co-stimulatory molecule, immunomodulatory molecule, chimeric antigen receptor, a transdomain negative mutant of a target protein, toxin, conditional toxin, antigen, transcription factor, structural protein, reporter protein, subcellular localization signal, tumour suppressor protein, growth factor, membrane protein, receptor, vasoactive protein or peptide, anti-viral protein or ribozyme, or a derivative thereof, or a micro-RNA.

In some embodiments, said nucleotide of interest encodes a molecule useful in the treatment of a disorder selected from the following:
(i) A disorder which responds to cytokine and cell proliferation/differentiation activity; immunosuppressant or immunostimulant activity (e.g. for treating immune deficiency, including infection with human immunodeficiency virus, regulation of lymphocyte growth; treating cancer and many autoimmune diseases, and to prevent transplant rejection or induce tumour immunity); regulation of haematopoiesis (e.g. treatment of myeloid or lymphoid diseases); promoting growth of bone, cartilage, tendon, ligament and nerve tissue (e.g. for healing wounds, treatment of burns, ulcers and periodontal disease and neurodegeneration); inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating, for example, septic shock or Crohn's disease); macrophage inhibitory and/or T cell inhibitory activity and thus, anti-inflammatory activity; anti-immune activity (i.e. inhibitory effects against a cellular and/or humoral immune response, including a response not associated with inflammation); inhibition of the ability of macrophages and T cells to adhere to extracellular matrix components and fibronectin, as well as up-regulated fas receptor expression in T cells;
(ii) Malignancy disorders, including cancer, leukaemia, benign and malignant tumour growth, invasion and spread, angiogenesis, metastases, ascites and malignant pleural effusion;
(iii) Autoimmune diseases including arthritis, including rheumatoid arthritis, hypersensitivity, allergic reactions, asthma, systemic lupus erythematosus, collagen diseases and other diseases;
(iv) Vascular diseases including arteriosclerosis, atherosclerotic heart disease, reperfusion injury, cardiac arrest, myocardial infarction, vascular inflammatory disorders, respiratory distress syndrome, cardiovascular effects, peripheral vascular disease, migraine and aspirin-dependent anti-thrombosis, stroke, cerebral ischaemia, ischaemic heart disease or other diseases;
(v) Diseases of the gastrointestinal tract including peptic ulcer, ulcerative colitis, Crohn's disease and other diseases;
(vi) Hepatic diseases including hepatic fibrosis, liver cirrhosis;
(vii) Inherited metabolic disorders including phenylketonuria PKU, Wilson disease, organic acidemias, urea cycle disorders, cholestasis, and other diseases;
(viii) Renal and urologic diseases including thyroiditis or other glandular diseases, glomerulonephritis or other diseases;
(ix) Ear, nose and throat disorders including otitis or other oto-rhino-laryngological diseases, dermatitis or other dermal diseases;
(x) Dental and oral disorders including periodontal diseases, periodontitis, gingivitis or other dental/oral diseases;
(xi) Testicular diseases including orchitis or epididimo-orchitis, infertility, orchidal trauma or other testicular diseases;
(xii) Gynaecological diseases including placental dysfunction, placental insufficiency, habitual abortion, eclampsia, pre-eclampsia, endometriosis and other gynaecological diseases;
(xiii) Ophthalmologic disorders such as Leber Congenital Amaurosis (LCA) including LCA10, posterior uveitis, intermediate uveitis, anterior uveitis, conjunctivitis, chorioretinitis, uveoretinitis, optic neuritis, glaucoma, including open angle glaucoma and juvenile congenital glaucoma, intraocular inflammation, e.g. retinitis or cystoid macular oedema, sympathetic ophthalmia, scleritis, retinitis pigmentosa, macular degeneration including age related macular degeneration (AMD) and juvenile macular degeneration including Best Disease, Best vitelliform macular degeneration, Stargardt's Disease, Usher's syndrome, Doyne's honeycomb retinal dystrophy, Sorby's Macular Dystrophy, Juvenile retinoschisis, Cone-Rod Dystrophy, Corneal Dystrophy, Fuch's Dystrophy, Leber's congenital amaurosis, Leber's hereditary optic neuropathy (LHON), Adie syndrome, Oguchi disease, degenerative fondus disease, ocular trauma, ocular inflammation caused by infection, proliferative vitreo-retinopathies, acute ischaemic optic neuropathy, excessive scarring, e.g. following glaucoma filtration operation, reaction against ocular implants, corneal transplant graft rejection, and other ophthalmic diseases, such as diabetic macular oedema, retinal vein occlusion, RLBP1-associated retinal dystrophy, choroideremia and achromatopsia;
(xiv) Neurological and neurodegenerative disorders including Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, strokes, post-polio syndrome, psychiatric disorders, myelitis, encephalitis, subacute sclerosing pan-encephalitis, encephalomyelitis, acute neuropathy, subacute neuropathy, chronic neuropathy, Fabry disease, Gaucher disease, Cystinosis, Pompe disease, metachromatic leukodystrophy, Wiscott Aldrich Syndrome, adrenoleukodystrophy, beta-thalassemia, sickle cell disease, Guillaim-Barre syndrome, Sydenham chorea, myasthenia gravis, pseudo-tumour cerebri, Down's Syndrome, Huntington's disease, CNS compression or CNS trauma or infections of the CNS, muscular atrophies and dystrophies, diseases, conditions or disorders of the central and peripheral nervous systems, motor neuron disease including amyotropic lateral sclerosis, spinal muscular atropy, spinal cord and avulsion injury; and
(xv) Cystic fibrosis, mucopolysaccharidosis including Sanfilipo syndrome A, Sanfilipo syndrome B, Sanfilipo syndrome C, Sanfilipo syndrome D, Hunter syndrome, Hurler-Scheie syndrome, Morquio syndrome, ADA-SCID, X-linked SCID, X-linked chronic granulomatous disease, porphyria, haemophilia A, haemophilia B, post-traumatic inflammation, haemorrhage, coagulation and acute phase response, cachexia, anorexia, acute infection, septic shock, infectious diseases, diabetes mellitus, complications or side effects of surgery, bone marrow transplantation or other transplantation complications and/or side effects, complications and side effects of gene therapy, e.g. due to infection with a viral carrier, or AIDS, to suppress or inhibit a humoral and/or cellular immune response, for the prevention and/or treatment of graft rejection in cases of transplantation of natural or artificial cells, tissue and organs such as cornea, bone marrow, organs, lenses, pacemakers, natural or artificial skin tissue.

In a further aspect, the invention provides a stable or transient production cell for producing lentiviral vectors comprising at least one nucleotide sequence encoding a modified U1 snRNA of the invention.

In a further aspect, the invention provides a method for producing a lentiviral vector as described herein, comprising the steps of:
a. introducing nucleotide sequences encoding vector components including gag, env, rev and the RNA genome of the lentiviral vector, and at least one nucleotide sequence encoding a modified U1 snRNA of the invention, into a cell;
b. optionally selecting for a cell which comprises said nucleotide sequences encoding vector components and at least one modified U1 snRNA;
c. culturing the cell under conditions in which said vector components are co-expressed with said modified U1 snRNA and the lentiviral vector is produced.

In a further aspect, the invention provides a lentiviral vector produced by the method of the invention.

In a further aspect, the invention provides the use of a modified U1 snRNA of the invention or an expression cassette of the invention for the production of a lentiviral vector.

In a further aspect, the invention provides a lentiviral vector produced in the presence of a modified U1 snRNA of the invention, wherein the lentiviral vector comprises an inactivated major splice donor site in the RNA genome of the lentiviral vector.

In some embodiments, said lentiviral vector is derived from HIV-1, HIV-2, SIV, FIV, BIV, EIAV, CAEV or Visna lentivirus.

In some embodiments, said lentiviral vector is derived from HIV-1, HIV-2 or EIAV.

In some embodiments, said lentiviral vector is derived from HIV-1.

In some embodiments, said lentiviral vector is derived from SIV.

In some embodiments as described herein, the major splice donor site in the RNA genome of the lentiviral vector is inactivated.

In some embodiments, the major splice donor site and the cryptic splice donor site 3′ to the major splice donor site in the RNA genome of the lentiviral vector are inactivated.

In some embodiments, said lentiviral vector is a third generation lentiviral vector.

In some embodiments, the major splice donor site in the RNA genome of the lentiviral vector is inactivated, and wherein the cryptic splice donor site 3′ to the major splice donor site is inactivated, wherein said nucleotide sequence is for use in a tat-independent lentiviral vector.

In some embodiments, the major splice donor site in the RNA genome of the lentiviral vector is inactivated, and wherein the cryptic splice donor site 3′ to the major splice donor site is inactivated, wherein said nucleotide sequence is produced in the absence of tat.

In some embodiments, the major splice donor site in the RNA genome of the lentiviral vector is inactivated, and wherein the cryptic splice donor site 3′ to the major splice donor site is inactivated, wherein said nucleotide sequence has been transcribed independently of tat.

In some embodiments, the major splice donor site in the RNA genome of the lentiviral vector is inactivated, and wherein the cryptic splice donor site 3′ to the major splice donor site is inactivated, wherein said nucleotide sequence is for use in a U3-independent lentiviral vector.

In some embodiments, the major splice donor site in the RNA genome of the lentiviral vector is inactivated, and wherein the cryptic splice donor site 3′ to the major splice donor site is inactivated, wherein said nucleotide sequence has been transcribed independently of the U3 promoter.

In some embodiments, the major splice donor site in the RNA genome of the lentiviral vector is inactivated, and wherein the cryptic splice donor site 3′ to the major splice donor site is inactivated, wherein said nucleotide sequence has been transcribed by a heterologous promoter.

In some embodiments, the cryptic splice donor site is the first cryptic splice donor site 3′ to the major splice donor site.

In some embodiments, said cryptic splice donor site is within 6 nucleotides of the major splice donor site.

In some embodiments, the major splice donor site and cryptic splice donor site are mutated or deleted.

In one aspect the invention provides a nucleotide sequence encoding the RNA genome of the lentiviral vector, wherein the nucleotide sequence prior to inactivation of the splice sites comprises a sequence as set forth in any of SEQ ID NOs: 1, 3, 4, 9, 10 and/or 13. The nucleotide sequence may comprise a sequence with a mutation or deletion relative to the sequence as set forth in any of SEQ ID NOs: 1, 3, 4, 9, 10 and/or 13. In one aspect the sequence comprises SEQ ID NO: 13.

In one aspect the nucleotide sequence comprises an inactivated major splice donor site which would otherwise have a cleavage site immediately upstream of nucleotide 1 of the major splice donor region (SEQ ID NO: 13).

In one aspect the nucleotide sequence comprises an inactivated major splice donor site and an inactivated cryptic splice donor site which would otherwise have a cleavage site immediately upstream of nucleotide 1, as well as between nucleotides 4 and 5 corresponding to nucleotides of the major splice donor region (SEQ ID NO: 13).

In some embodiments, the nucleotide sequence encoding the RNA genome of the lentiviral vector comprises an inactivated major splice donor site which would otherwise have a cleavage site between nucleotides corresponding to nucleotides 13 and 14 of SEQ ID NO: 1.

In some embodiments, the nucleotide sequence of the major splice donor site prior to inactivation comprises the sequence as set forth in SEQ ID NO: 4.

In some embodiments, the nucleotide sequence of the cryptic splice donor site prior to inactivation comprises the sequence as set forth in SEQ ID NO: 10.

In some embodiments, the nucleotide sequence encoding the RNA genome of the lentiviral vector comprises an inactivated cryptic splice donor site which would otherwise have a cleavage site between nucleotides corresponding to nucleotides 17 and 18 of SEQ ID NO: 1.

In some embodiments, the nucleotide sequence encoding the RNA genome of the lentiviral vector comprises a sequence as set forth in any of SEQ ID NOs: 2, 5, 6, 7, 8, 11, 12 and/or 14.

In a preferred aspect the nucleotide sequence comprises a sequence as set forth in SEQ ID NO: 14.

In some embodiments, the nucleotide sequence encoding the RNA genome of the lentiviral vector does not comprise a sequence as set forth in SEQ ID NO:9.

In some embodiments, the splicing activity from the major splice donor site and cryptic splice donor site of the RNA genome of the lentiviral vector is suppressed or ablated.

In some embodiments, the splicing activity from the major splice donor site and cryptic splice donor site of the RNA genome of the lentiviral vector is suppressed or ablated in transfected cells or in transduced cells.

In some embodiments, the nucleotide sequence encoding the RNA genome of the lentiviral vector is operably linked to the nucleotide sequence encoding the modified U1 snRNA.

In one aspect the nucleotide sequence encoding a modified U1 snRNA is on a different nucleotide sequence, for example on a different plasmid, to the nucleotide sequence encoding the RNA genome of the lentiviral vector.

In one aspect the nucleotide sequence according to the invention is for use in a tat-independent lentiviral vector system. In one aspect the lentiviral vector system may be a 3^{rd} generation lentiviral vector system.

In one aspect nucleotide sequence may be suitable for use in a lentiviral vector in a tat-independent system for vector production. As described herein, 3^{rd} generation lentiviral vectors are tat-independent, and the nucleotide sequences according to the present invention may be used in the context of a 3^{rd} generation lentiviral vector. For clarity it is understood that the term ‘tat-independent’ means that the HIV-1 U3 promoter used to drive transcription of the vector genome cassette is replaced by a heterologous promoter. In one aspect of the invention tat is not provided in the lentiviral vector production system, for example tat is not provided in trans. In one aspect the cell or vector or vector production system as described herein does not comprise the tat protein.

### BRIEF DESCRIPTION OF THE FIGURES

FIG. 1: A schematic of a U1 snRNA molecule and an example of how to modify the targeting sequence for use in the invention. The endogenous non-coding RNA, U1 snRNA binds to the consensus splice donor site (5′-MAGGURR-3′) via the 5′-(AC)UUACCUG-3′ (grey highlighted) native splice donor targeting sequence during early steps of intron splicing. Stem loop I binds to U1A-70K protein that has been shown to be important for polyA suppression. Stem loop II binds to U1A protein, and the 5′-AUUUGUGG-3′ sequence binds to Sm proteins, which together with Stem loop IV, is important for U1 snRNA processing. In the invention, the modified U1 snRNA is modified to introduce a heterologous sequence that is complementary to a target sequence within the vector genome vRNA molecule at the site of the native splice donor targeting sequence; in this figure the example given directs the modified U1 snRNA to 15 nucleotides (256-270 relative to the first nucleotide of the vector genome molecule, 256U1) of a standard HIV-1 lentiviral vector genome (located in the SL1 loop if the packaging signal).

FIG. 2: The increase in lentiviral vector titres mediated by modified U1 snRNAs is independent of polyA site suppression in the 5′LTR of the vector genome. [A] A GFP-polyA-GLuciferase reporter cassette was designed to assess the impact of two polyA signal mutants (pAM1=AAUAAA>AACAAA; pAKO=deletion of AAUAAA) and the wild type polyA signal (wt pA=AAUAAA) on transcriptional read-through the HIV-1 polyA site. Read-through the HIV-1 polyA signal was measurable by luciferase activity, which was normalized by GFP expression. [B] A standard lentiviral vector genome (STD-LV), and two lentiviral vector genomes containing different 5′LTR polyA signal mutants (Δ5′ pA-LV pAM1 or pAM2), were used to make vector particles either in the absence (NegCtrl) or presence of a modified U1 snRNA (256_U1, supplied in parallel during production), and then titrated.

FIG. 3: The increase in lentiviral vector titres mediated by modified U1 snRNAs does not require functional U1A-70K or U1A protein binding loops. [A] U1 snRNAs were modified to target the 256-270 region of an LV genome or two sites in a lacZ sequence (negative control). Modified U1A snRNAs were made with mutations known to disrupt U1A-70K protein, U1A protein or sm protein binding (sequences displayed). Modified U1A5, U1A6 and U1A7 snRNA variants were also made. [B] Impact on LV-GFP titres by the various modified U1 snRNAs supplied in trans during production.

FIG. 4: Effect of changing the length and the targeting sequence with modified U1 snRNAs. A standard lentiviral vector encoding GFP was produced in the absence (black bar) or the presence of modified U1 snRNAs with targeting sequence to sites along the length of the 5′end of the vector genome vRNA molecule comprising either 15 nucleotide (dark grey bar) or 9 nucleotide (light grey bar) targeting lengths of complementarity. Modified U1 snRNAs are named according to the first nucleotide of the targeting sequence site along the length of the 5′end of the vector genome vRNA molecule. The data bars for each modified U1 snRNA are aligned underneath the approximate labelled position of each known functional sequence within the 5′end of the vector genome vRNA (not to scale).

FIG. 5: A standard lentiviral vector encoding GFP was produced in the absence (black bar) or the presence of modified U1 snRNAs with targeting sequence to sites along the length of the 5′end of the vector genome vRNA molecule comprising 15 nucleotide (dark grey bar) targeting lengths of complementarity. Modified U1 snRNAs are named according to the first nucleotide of the targeting sequence site along the length of the 5′end of the vector genome vRNA molecule. The data bars for each modified U1 snRNA are aligned underneath the approximate labelled position of each known functional sequence within the 5′end of the vector genome vRNA (not to scale).

FIG. 6: Increasing lentiviral vector titres encoding different transgenes using modified U1 snRNAs. Standard lentiviral vectors encoding GFP (pHIV-EF1a-GFP) or a chimeric antigen receptor to CD19 (pHIV-EF1a-CD19) were produced in the absence (−) or the presence of modified U1 snRNAs targeting either sites within the lentiviral vector packaging region (256U1 or 305U1) or a LacZ control (LacZU1). Modified U1 snRNAs are named according to the first nucleotide of the targeting sequence site within the lentiviral vector packaging region. The modified U1 snRNA expression constructs were supplied at two different doses: 1× or 4×.

FIG. 7: Implications of aberrant splicing from the major splice donor site (MSD) within HIV-1 based lentiviral vectors. A. A schematic to show the typical configuration of a third generation (Self-inactivating (SIN)) lentiviral vector expression cassette, containing a functional major splice donor embedded within stem loop (SL2) of the packaging signal, and the types of mRNA generated during lentiviral vector production. The types of mRNA generated from a ‘standard’ lentiviral vector (LV) DNA cassette and a lentiviral vector DNA cassette with (a) functional mutation(s) in the MSD region (‘MSD-KO LV DNA cassette’) that suppress or ablate the promiscuous activity from the MSD are shown. For both cassettes, the full-length (‘Unspliced’) vector RNA (vRNA) results from the co-expression of rev, which binds to the rev response element (RRE), and is generally believed to repress splicing from the MSD to splice acceptor 7 (sa7) included with the RRE sequence. For a standard lentiviral vector DNA cassette, in the absence of rev, it is generally believed that splicing-out of all introns occurs efficiently (′Spliced). However, ‘aberrant’ splice products can be made during lentiviral vector production wherein the MSD highly efficiently splices to splice acceptor sites or cryptic splice acceptor sites (‘‘Aberrant’ spliced’), typically ‘over-looking’ the RRE-containing intron such that rev has minimal impact on this activity of the MSD. Lentiviral vector production can also be performed with co-expression of modified U1 snRNAs redirected to the packaging region of MSD-mutated lentiviral vector DNA cassettes. (Key: Pro, promoter; region from 5′R to gag contains the packaging element {ψ}; msd, major splice donor; cppt, central polypurine tract; Int, intron; sd/sa, splice donor/acceptor; GOI, gene of interest; grey arrow indicate position of forward {f} and reverse {r} primers to assess the proportion of Unspliced vRNA produced during 3^{rd} generation lentiviral vector production. Post-transcriptional regulatory element {PRE} not shown for clarity). B. Standard 3^{rd} generation lentiviral vector production was performed +/−rev in HEK293T cells and total RNA extracted from post-production cells. Total RNA was subjected to qPCR (SYBR green) using two primer sets (position marked in A): f+rT amplified total transcripts generated from the lentiviral vector expression cassette, and f+rUS amplified Unspliced transcripts; therefore the proportion of Unspliced-to-Total vRNA transcripts were calculated and plotted. The data indicates that the proportion of Unspliced vRNA relative to total during standard 3^{rd} generation lentiviral vector production is modest and varies according to the internal transgene cassette (in this case containing different promoters and the GFP gene); moreover, this proportion is only minimally increased by the action of rev.

FIG. 8: HIV-1 lentiviral vector genomes containing three different promoter-GFP expression cassettes (EF1a, EFS and CMV) were modified to functionally mutate the MSD resulting in the ‘MSD-2KO’ lentiviral vector genomes or back-bones (see FIG. 15A for description of mutations). Vectors were produced in HEK293T cells under standard protocol and titrated. The data shows that the functional mutation of the MSD (‘MSD-2KO’) results in up to 100-fold reduction in lentiviral vector titres.

FIG. 9: A A schematic to show the configuration of standard or MSD-mutated lentiviral vector expression cassettes encoding an EF1a-GFP internal expression cassette, and the types of mRNA generated during lentiviral vector production. (Key: Pro, promoter; region from 5′R to gag contains the packaging element {ψ}; msd, major splice donor; cppt, central polypurine tract; Int, intron; sd/sa, splice donor/acceptor; GOI, gene of interest; grey arrow indicate position of forward {f} and reverse {r} primers to assess the proportion of Unspliced vRNA produced during 3^{rd} generation lentiviral vector production. Post-transcriptional regulatory element {PRE} not shown for clarity). B i The standard lentiviral vectors or MSD-2KO lentiviral vectors were produced in HEK293T cells+/−tat, or 179U1, or 305U1, and titrated. ii Total cytoplasmic mRNA was extracted from post-production cells and analysed by RT-PCR/gel electrophoreses using primers (f+rG) that could detect the main ‘aberrant’ splice product from the SL2 splicing region SL2 splicing region to the EF1a splice acceptor. The data show that modified U1 snRNAs redirected to the 5′ packaging region of MSD-2KO lentiviral vector genome (vRNA) were able to increase titres of both standard and MSD-2KO lentiviral vectors in a manner similar to tat. The MSD-2KO mutation abolished detection of the ‘aberrant’ splice product, which is from the SL2 splicing region to the EF1a splice acceptor (see FIG. 9A). Importantly, the increase in titres by the modified U1 snRNAs was accompanied by maintenance of virtually undetectable ‘aberrant’ spliced product, in contrast to the use of tat.

FIG. 10: Standard lentiviral vectors or MSD-mutated lentiviral vectors encoding a GFP internal cassette driven by EF1a, EFS or CMV promoters were produced in HEK293T cells+/−256U1, and titrated. Enhanced lentiviral vector titres by the use of modified U1 snRNAs redirected to the 5′ packaging region are independent of promoter employed within the transgene cassette. The data shows that, in large part, the attenuating phenotype of the MSD-2KO mutation is rescued by the co-expression of modified U1 snRNA, and therefore this surprisingly boosts titre of MSD-mutated lentiviral vector genomes disproportionately compared to standard lentiviral vector genomes.

FIG. 11: Enhancement of MSD-mutated lentiviral vector titres by the use of modified U1 snRNAs redirected to the 5′ packaging region is not linked to suppression of potential activity of the 5′polyA signal within the 5′LTR. Previous reports show that mutation of the MSD can activate the polyA signal within the 5′R sequence of the 5′LTR of HIV-1 provirus and ‘mini-reporter’ cassettes leading to premature termination of transcription; the binding of endogenous U1 snRNA and even redirected U1 snRNAs could block this polyA activity. A A GFP-polyA-GLuciferase reporter cassette was designed to assess the impact of two polyA signal mutants (pAM1=AAUAAA>AACAAA; pAKO=deletion of AAUAAA) and the wild type polyA signal (wt pA=AAUAAA) on transcriptional read-through the HIV-1 polyA site. Read-through the HIV-1 polyA signal was measurable by luciferase activity, which was normalized by GFP expression. B To test if modified U1 snRNAs were acting in a similar manner, a functional polyA mutation (pAm1) in the 5′polyA signal was introduced into MSD-mutated lentiviral vector genomes harbouring EF1a-GFP or CMV-GFP expression cassettes. Standard and MSD-mutated lentiviral vector genomes harbouring EF1a-GFP or CMV-GFP expression cassettes were also used. Lentiviral vectors were produced in HEK293T cells+/−305U1, and titrated. The data indicate that functional ablation of the 5′polyA signal only led to a very modest increase in lentiviral vector titres, and therefore the observed increase in lentiviral vector titres afforded by the modified U1 snRNA—especially that of the MSD-2KO/polyA-mutated lentiviral vector genome—could not be attributed to suppression of 5′polyA activity.

FIG. 12: Several mutations were introduced into 305U1 and 256U1 modified U1 snRNAs, that are known to ablate U1-70K protein binding to SL1, U1A protein binding to SL2 or Sm protein binding to/near SL4 of the vector genome. Standard or MSD-2KO lentiviral vectors encoding an EF1a-GFP internal cassette were produced in the presence of these mutated modified U1 snRNAs and titrated, and titre values normalized to standard lentiviral vectors produced without modified U1 snRNAs. The data show that the enhancement in MSD-2KO lentiviral vector titres by modified U1 snRNAs is not dependent on U1-70K protein or U1A protein binding but is dependent on the Sm protein binding site. Thus, enhancement of MSD-2KO lentiviral vector titres by the use of modified U1 snRNAs redirected to the 5′ packaging region is not linked to any known function of U1 snRNA.

FIG. 13: Enhancement of MSD-mutated lentiviral vector titres by the use of modified U1 snRNAs containing targeting sequence of varying lengths. MSD-2KO lentiviral vectors containing the EF1a-GFP cassette were produced in HEK293T cells in the presence of modified U1 snRNAs targeting the ‘305’ region, wherein each modified U1 snRNA comprised a re-targeting sequence of different length in complementarity. The titre increase was observed when using modified U1 snRNAs containing complementarity lengths of 7-to-15 nucleotides, with maximal effect observed at 10 nucleotides or more.

FIG. 14: Maximal titre recovery/boost of an MSD-mutated lentiviral vector is observed when targeting modified U1 snRNAs to the packaging region of the vector genome RNA. An MSD-2KO lentiviral vector containing the EF1-GFP cassette was produced in the presence of modified U1 snRNAs with targeting sequence to sites along the length of the 5′end of the vector genome vRNA molecule comprising 15 nucleotide lengths of complementarity (or 9 nucleotides where indicated). Modified U1 snRNAs are named according to the first nucleotide of the targeting sequence site along the length of the 5′end of the vector genome vRNA molecule. The data bars for each modified U1 snRNA are aligned underneath the approximate labelled position of each known functional sequence within the 5′end of the vector genome vRNA (not to scale).

FIG. 15: A description of functional major splice donor mutations, their impact on lentiviral vector titres, and recovery by modified U1 snRNA. A The sequence of the stem loop 2 (SL2) region of ‘wild type’ HIV-1 (NL4-3; the ‘standard’ sequence within current lentiviral vector genomes) is shown at the top. The sequence comprises the major splice donor site (MSD: consensus=CTGGT) and a cryptic splice donor site (that is utilized when the MSD site is mutated on its own (crSD: consensus=TGAGT). The nucleotides at the position of splicing when the splice donor site is used are identified in bold and by arrows. Four functional MSD mutations that ablate both the MSD and the crSD site splicing activities are described: MSD-2KO, which mutates the two ‘GT’ motifs from the MSD and the crSD sites (and is used widely in most Examples); MSD-2KOv2, which also comprises mutations that ablate both the MSD and crSD sites; MSD-2KOm5, which introduces an entirely new stem-loop structure lacking any splice donor sites; and ΔSL2, which deletes the SL2 sequence entirely. The substitutions introduced to the SL2 sequence in the MSD-2KO,MSD-2KOv2 and MSD-2KOm5 mutations are shown in lowercase italics. B The four lentiviral vector genome variants comprising functional MSD mutations (described in FIG. 15A) were cloned with EFS-GFP internal cassettes, and MSD-2KO or MSD-2KOm5 variants additionally cloned with EF1a-, CMV- or huPGK-GFP internal cassettes. Standard and MSD-mutated LVs were produced in HEK293T cells+/−256U1, and titrated. The data indicates that the degree of attenuation of lentiviral vector titre can vary according to the specific mutation, and that the MSD-2KOm5 variant generally produced a less attenuated phenotype. The modified U1 snRNA was capable of increasing lentiviral vector titres for the four lentiviral vector genome variants comprising functional MSD mutations when co-expressed during production. Titre increases were greatest when the 256U1 was expressed with MSD-mutated LV genomes harbouring the MSD-2KOm5 sequence.

FIG. 16: The modified U1 snRNA expression cassette can be located to the lentiviral vector genome plasmid backbone for ease of use in transient transfection protocols. Many of the Examples use a separate modified U1 snRNA expressing plasmid in co-transfection with lentiviral vector component plasmids in production of lentiviral vectors. To identify ‘permissive’ sites on the lentiviral vector genome plasmid backbone in order to be able to provide the modified U1 snRNA cassette in cis during transient transfection, three variants were cloned. A A schematic of the lentiviral vector genome variants providing the modified U1 snRNA cassette in cis during transient transfection. Version 1 (‘[cis] ver’) and version 3 (′[cis] ver3′) placed the modified U1 snRNA cassette between the resistance marker and the origin of replication such that the modified U1 snRNA cassette was inverted relative to the lentiviral vector genome cassette (the resistance marker orientation differed between ver1 and ver3), and version 2 (‘[cis] ver2’) placed the modified U1 snRNA cassette upstream and in the same orientation of the lentiviral vector genome cassette. (Key: Pro, promoter; region from 5′R to gag contains the packaging element {ψ}; msd, major splice donor {here shown as MSD-2KO}; RRE, rev response element; cppt, central polypurine tract; Transgenic, heterologous sequence comprising therapeutic payload; U1-Pro, U1 promoter; Term[3′box], U1 transcriptional terminator). B The three ‘cis’ versions of the MSD-2KO lentiviral vector genome plasmid containing an EF1a-GFP cassette were used to produce lentiviral vectors in HEK293T cells in parallel to the ‘trans’ approach, where the same MSD-2KO lentiviral vector genome (without inserted modified U1 snRNA cassette in the backbone) was produced+/−modified U1 snRNA supplied by co-transfection with a separate plasmid. The data shows that similarly to co-transfection of a separate modified U1 snRNA encoding plasmid, MSD-2KO lentiviral vector titre could be increased by use of the ‘cis’ lentiviral vector genomes.

FIG. 17: Further demonstration of use of modified U1 snRNA to increase titres of standard lentiviral vectors encoding therapeutic transgenes. Standard lentiviral vector vectors containing EF1a-driven transgene cassettes encoding codon-optimised or wild type human alpha1-antitrypsin fused to GFP via a T2A peptide, or a chimeric antigen receptor (CAR) to the cancer antigen 5T4, where produced in serum-free, suspension HEK293T cells+/−modified U1 snRNA (256U1), and titrated by Integration assay and GFP-FACS assay (where denoted). The data shows a ˜3-fold increase in vector titres when the modified U1 snRNA is supplied during production.

FIG. 18: Successful isolation of HEK293T cells stably expressing modified U1 snRNA that enables increase of standard or MSD-2KO lentiviral vectors demonstrates that the modified U1 snRNA cassettes can be introduced into lentiviral vector packaging and producer cell lines. Standard or MSD-2KO lentiviral vector genomes containing EFS-GFP cassettes were produced in HEK293T or HEK293T.305U1 (9nt variant) cells+/−additional 305U1 plasmid. The data indicate that stable cassettes expressing modified U1 snRNA can be introduced into cells without toxicity.

FIG. 19: Aberrantly spliced mRNA expressing transgene during lentiviral vector production is abolished in MSD-2KO lentiviral vectors, reducing the amount of transgene mRNA required to be targeted by TRAP when utilising the TRiP system. A A schematic of a ‘TRiP’ lentiviral vector genome encoding an EF1a-GFP transgene cassette, wherein the TRAP binding site (tbs) is positioned within the 5′UTR of the cassette (supply of TRAP during vector production reduces transgene expression levels). During production of MSD-2KO lentiviral vectors, the full length, unspliced packagable vRNA and transgene mRNA are main forms of RNA produced from the lentiviral vector cassette (i) (when the transgene promoter is active during production). However, the promiscuous activity of the MSD in standard lentiviral vector genomes leads to additional ‘aberrant’ splice products that may encode the transgene (ii); this could occur independently of the internal transgene promoter i.e. a tissue specific promoter. (Key: Pro, promoter; region from 5′R to gag contains the packaging element {ψ}; msd, major splice donor; cppt, central polypurine tract; Int, intron; sd/sa, splice donor/acceptor; GOI, gene of interest; grey arrow indicate position of forward {f} and reverse {r} primers to assess the proportion of Unspliced vRNA produced during 3^{rd} generation lentiviral vector production. Post-transcriptional regulatory element {PRE} not shown for clarity). B Standard or MSD-2KO lentiviral vector genome plasmid containing an EF1a-GFP cassette were used to produce lentiviral vectors in HEK293T cells, and GFP expression scores generated (% GFP×MFI). Relative to the total amount of GFP produced in cultures during standard lentiviral vector production, the MSD-2KO had the substantial effect of reducing the amount of GFP produced even in the absence of TRAP. Accordingly, the repressive effects of TRAP were augmented by use of the MSD-2KO lentiviral vector genome, leading to much lower levels of GFP in cultures.

FIG. 20: The enhancement of lentiviral vector titres by use of modified U1 snRNAs is correlated with increased vector RNA packaging into virions. Total RNA was extracted from RNAse-treated crude vector supernatant of vectors produced in Example 5 (see FIG. 6). Purified RNA was subjected to RT-qPCR using primers against the HIV-1 packaging signal in the vector genome RNA. The increase in vRNA signal within crude vector harvest produced in the presence of modified U1 snRNA directed against the vRNA was of a similar magnitude to the increase in vector titres reported in FIG. 6.

FIG. 21: Enhancement of α1-anti-trypsin (α1AT) encoding VSVG-pseudotyped lentiviral vector titres by use of modified U1 snRNAs, and accompanying suppression of transgene expression during production. HIV-1 based LVs encoding codon-optimised (co) or wild type (wt) α1AT proteins translationally linked to GFP expression (by T2A peptide) were produced in HEK293T serum-free, suspension cells in the absence or presence of 256U1. [A] Clarified vector harvests were titrated by transduction of adherent HEK293 Ts and flow cytometry. [B] Post-production cell lysates were immunoblotted for transgene proteins and β-actin. A modest but observable reduction in transgene expression was apparent in cells co-transfected with 256U1 expression plasmid.

FIG. 22: Enhancement of α1-anti-trypsin (α1AT) encoding Sendai virus (SeV) envelope-pseudotyped lentiviral vector titres by use of modified U1 snRNAs, and accompanying suppression of transgene expression during production. HIV-1 based LVs encoding α1AT protein translationally linked to GFP expression (by T2A peptide) were produced in adherent or suspension (serum-free) HEK293T cells in the absence or presence of 256U1. SeV-F/HN pseudotyped LVs required activation by tryspin-treatment prior to transduction. [A] Clarified vector harvests were titrated by transduction of adherent HEK293 Ts and flow cytometry. [B] Post-production cell lysates were immunoblotted for transgene proteins and β-actin. A modest but observable reduction in transgene expression was apparent in cells co-transfected with 256U1 expression plasmid. The increase in LV titre mediated by 256U1 was more profound for SeV F/HN-pseudotyped vector compared to VSVG-pseudotyped vector (FIG. 21) due to the reduction in α1-anti-trypsin expressed during production. The SeV F protein requires activation by trypsin prior to transduction, and so the presence of a1AT within crude vector presumably inhibits this activation step.

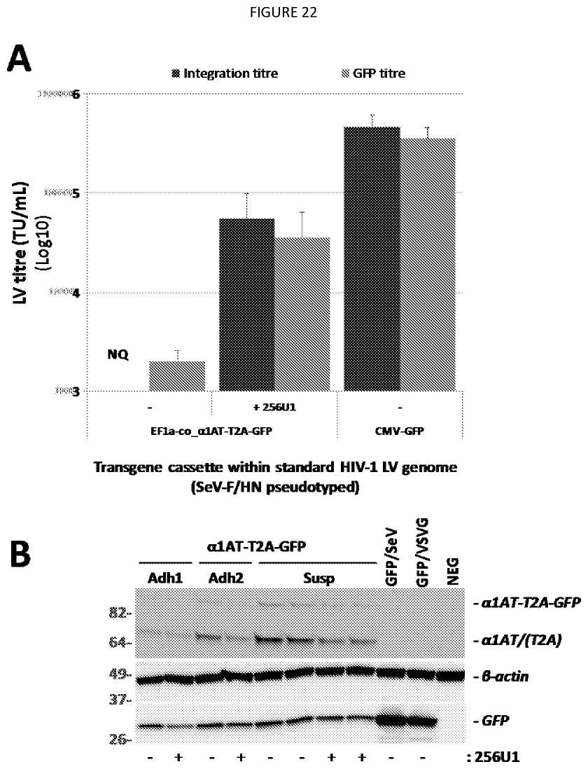

FIG. 23: Use of modified U1 snRNAs to increase titres of a lentiviral vector containing inverted transgene cassette. [A] A schematic of an LV genome expression cassette encoding an inverted β-globin gene (containing the required exons/introns for efficient expression in primary cells), driven by an LCR-β-globin promoter. [B] LV preps were produced in suspension (serum-free) HEK293T cells in the absence or presence of the stated modified U1 snRNAs. Clarified vector harvests were titrated by transduction of adherent HEK293 Ts followed by integration assay.

FIG. 24: The increase in titres of a lentiviral vector by use of modified U1 snRNA is observed in concentrated vector material as well as in crude vector material. An HIV-1 based lentiviral vector encoding an EF1a promoter-driven firefly luciferase/GFP double reporter cassette was produced in suspension (serum-free) HEK293T cells in the absence or presence of 256U1. The bulk of clarified vector harvest was concentrated by centrifugation, achieving a ˜20-fold concentration factor. Clarified vector harvest and concentrated vector was titrated by transduction of adherent HEK293T cells followed by flow cytometry to measure GFP-positive cells.

FIG. 25: Development of a Taqman-based RT-qPCR assay for the detection of modified U1 snRNA to assess expression level and residuals. [A] A schematic to show the 256U1 snRNA molecule and the binding positions of the forward/reverse primers and the FAM/TAMRA conjugated probe. The primary difference between endogenous U1 snRNA and the modified U1 snRNAs described in the invention is the 5′end of the molecule; the native splice donor annealing sequence is replaced with a targeting sequence enabling the modified U1 snRNA to anneal to the vector genome RNA. Due to the necessary requirement to perform the cDNA synthesis step (using reverse transcriptase) using a reverse primer, both endogenous and modified U1 snRNAs will contribute to the pool of cDNA generated during cDNA synthesis of cellular RNA and (potentially) RNA extracted from vector particles. Therefore, to differentiate between the endogenous and modified U1 snRNAs, the forward primer is designed to anneal to the vRNA targeting sequence at the 5′end. [B] Plotted Ct-threshold values resulting from Taqman qPCR (+/−RT step) of total RNA extracted from cell lysates resulting from transfections with or without p256U1. The standard curve (black circles) was generated from p256U1 samples (2×10⁷ copies per reaction, serially 10-fold diluted), resulting in good range and linearity. The ˜2-fold cycle difference between non-transfected cell sample and p256U1-transfected cells (no RT step), indicates the likely level of residual p256U1 DNA associated with samples, despite samples being treated with DNAse post-RT step. The average difference in Cts between +RT and −RT treated samples derived from p256U1-transfected cells was 19.4 cycles, and at 80-fold dilution of samples (i.e. approximately 10⁵ copies per reaction) this difference was 20 cycles.

FIG. 26: The dose response of addition of modified U1 snRNA expression plasmid to the transfection mix during the transient transfection method of lentiviral vector production. An HIV-1 based LV-CAR vector (encoding an EF1a promoter-driven cassette expressing a CAR targeting to CD19) was produced in suspension (serum-free) HEK293T cells by transient transfection in the absence or presence of increasing amounts of co-transfected p256U1. [A] Post-production cells were subjected to total RNA extraction and the levels of vector genome RNA (vRNA) or 256U1 snRNA were quantified by RT-qPCR. All data were adjusted based on a control RT-qPCR performed against an endogenous transcript (RPH1). [B] Clarified LV-CAR vector supernatant was titrated by transduction of adherent HEK293T cells, followed by integration assay (qPCR of extracted host cell DNA using vRNA primers). The data indicate a correlation between 256U1 snRNA and vRNA levels within the production cells, leading to similar increases in output vector titres.

FIG. 27: An example of application of multi-variance modelling (by ‘Design of Experiment’ [DoE]) to optimise ratios of modified U1 snRNA and LV component plasmids for production of lentiviral vectors encoding a therapeutic transgene. HIV-EF1a-5T4CAR vector was produced at 40 mL shake flask scale in suspension (serum-free) HEK293T cells. GagPol and rev input levels were fixed, and genome, VSVG and 256U1 plasmid levels altered. Clarified crude harvest vector was titrated by transduction of adherent HEK293T cells followed by immune-flow cytometry using an anti-CAR antibody (light grey bars; ‘Test’ T1-28). The titre values generated by the DoE experiment enabled prediction of an optimal level of p256U1 input, and then further vector preps were produced with either this input level (‘DoE-predicted’ D1-3) or without p256U1 (‘Platform’ P1-3). This optimisation experiment enabled p256U1 to be applied resulting in ˜10-fold increase in HIV-EF1a-5T4CAR output titres.

FIG. 28: Production and concentration of HIV-EF1a-5T4CAR vector+/−256U1 to generate concentrated vector for the transduction of primary Tcells and residual 256U1 snRNA analysis. HIV-EF1a-5T4CAR (‘LV-CAR’) vector was produced by transient transfection of suspension (serum-free) HEK293T cells at 250 mL shake flask scale and subjected to ion-exchange chromatography, DNAse treatment by salt active nuclease (SAN), followed by low speed centrifugation. Vector preps were titrated by transduction of HEK293T cells followed by integration assay.

FIG. 29 Detection and quantification of residual 265U1 snRNA within vector preparations. Vector sample from production of HIV-EF1a-5T4CAR (‘LV-CAR’) produced+/−256U1 (see FIG. 28) were subjected to total RNA extraction before RT-qPCR analysis of vector-associated RNA to quantify vRNA and 256U1 residual DNA levels/ratios. The data demonstrate that the initially high levels of 256U1 snRNA detected in clarified vector harvest was mainly due to ‘free’ 256U1 snRNA that could be cleared by the treatment with Benzonase (Benzonase was not used to treat clarified vector harvest during purification). This treatment reduced the ratio of 256U1-to-vRNA to 1:20, and this was further reduced to 1:32 after salt active nuclease (SAN) treatment during downstream processing/concentration.

FIG. 30: Comparative protein analysis of concentrated/purified preparations of HIV-EF1a-5T4CAR produced+/−256U1 by mass spectrometry. The concentrated/purified LV-CAR preparations (see FIGS. 28 & 29) produced to transduce primary T-cells (see Table IV) were analysed by mass spectrometry to evaluate any major differences in protein content that might be caused by expression of the 256U1 modified U1 snRNA molecule during LV production. The top 400 protein hits for vector produced in the presence of 256U1 (LV-CAR[+256U1]) were ranked 1-400 based on relative abundance as a percentage total, and the relative abundances of these hits within LV-CAR or LV-CAR[+256U1] plotted on the y axis. Of the 400 proteins, the top 100 constituted ˜70% and the top 10 constituted ˜30% of the total protein abundance. The top two most abundant hits were Gag and VSV-G for both LV preps, and other cellular factors known to be incorporated at high levels in HIV-1 virions included Basigin, HSPc-71K, Agrin and Cyclophilin A, the latter specifically binds to the Capsid. The comparison demonstrated very little difference between the protein make-up of the two LV preps. The ratio of abundance of peptides mapping to Gag versus Pol was ˜16 for both LV preps, consistent with the expected ratio of ˜20 for HIV-1, demonstrating that the data was of good quality.

FIG. 31: Generation of CAR-T cells using HIV-EF1a-5T4CAR vector produced+/−256U1. Approximately 1.5×10⁶ peripheral blood mononuclear cells (PBMCs) from three healthy donors were cultured with CD3/CD28 T Cell Expander beads and incubated with IL-2. Activated T-cells were transduced with concentrated vector samples IV-CAR′ and IV-CAR[+256U1] (see FIGS. 28-30 & Table IV) at MOI 1.25, and additionally at MOI 0.3 for LV-CAR[+256U1]. [A] Total viable cell counts were monitored to day 13 post-transduction (D13) before frozen viable cell banks (1×10⁷ vc/vial) were made. CAR-Tcells were revived and expanded for 5 days (R+5) ready for cell-killing and cytokine release assays (see FIGS. 32 & 33). [B] Percentage transduction was measured at day8 (D8) post-transduction and upon revival from frozen stocks.

FIG. 32: Evaluation of functionality of CAR-T cells generated using LV-CAR vectors produced with or without 256U1; cytokine release. Revived CAR-T cells were expanded for a further 5 days and viabilities were all >97%. Approximately 1×10⁵ CAR-T cells were co-cultured with an equal number of target cell lines: THP-1, Kasumi-1 and SKOV-3 (all 5T4-positive), and AML-193, a 5T4 negative cell line. After 24 hours, culture supernatant was analysed using cytometric beads for Granzyme-B [A] and Interferon-v activity [B].

FIG. 33: Evaluation of functionality of CAR-T cells generated using LV-CAR vectors produced with or without 256U1; target cell killing. Revived CAR-T cells were expanded for a further 5 days and viabilities were all >97%. Approximately 1×10⁵ CAR-T cells were co-cultured with an equal number of target cell lines: THP-1, Kasumi-1 and SKOV-3 (all 5T4-positive), and AML-193, a 5T4 negative cell line. Target cells were labelled with a fluorescent cell tracing dye to allow subsequent identification by flow cytometry. After 40 hours cells were harvested and stained with a fluorescent viability dye. The percentage of non-viable target cells in each experimental well was measured by flow cytometry, and compared to the viabilities of the target cell-only cultures (No CAR-T added).

FIG. 34: Analysis of residual, vector-associated RNA in CAR-T expansion cultures after transduction with HIV-EF1a-5T4CAR produced in the presence of 256U1. During CAR-T cell expansion after transduction at two different MOIs with HIV-EF1a-5T4CAR (+256U1; see FIG. 31), cell pellets were harvested at day 8 and day 13. Total RNA was extracted and RT-qPCR was performed against RPH1 mRNA, vRNA (Psi) and 256U1 snRNA. The difference in abundances of the endogenous RPH1 transcripts and residual 256U1 snRNA was calculated by delta-Ct method.

FIG. 35: Production of HIV-EF1a-CAR(CD19) vectors by transient transfection of a lentiviral vector packaging cell line with or without p256U1 in shake flasks and bioreactors. Either HIV-EF1a-CAR_CD19 or HIV-EF1a-CAR_CD19-T2A-GFP genome plasmids were transfected into a suspension, serum-free adapted lentiviral vector packaging cell line (PAC) with or without p256U1. Production was performed either in 40 mL shake flasks or in 250 mL bioreactors. Suspension, serum-free adapted HEK293T cells were used as controls, wherein all vector component plasmid DNAs were also co-transfected.

FIG. 36: Enhancement of lentiviral vector production from a suspension (serum-free) adapted HEK293T cell line stably transfected with a 256U1 snRNA expression cassette. The suspension (serum-free) adapted HEK293T cell line ‘256U1c39’ was isolated from HEK293T cells stably transfected with a 256U1 snRNA expression cassette operably linked to a Hygromycin-B resistance marker cassette. The enhancement of HIV-EF1a-5T4CAR titres compared to the parental HEK293T cell line was assessed over 10 weeks, with or without selection pressure. The data demonstrate that the 256U1c39 clone stably produced 256U1 snRNA to levels close to or at the same levels at transiently transfected HEK293T parental cells.

FIG. 37: Testing retargeting sequence length of modified U1 snRNAs. HIV-EF1a-GFP, HIV-EF1a-CARCD19 or HIV-EF1a-5T4CAR vectors were produced in suspension (serum-free) HEK293T cells in the presence of modified U1 snRNAs targeted to position 305 within the LV packaging region of the vRNA. Each 305U1 variant tested contained a targeting sequence of varying length: 5, 7, 9-15 nucleotides and was compared to no modified U1 and 256U1 (15nt). Clarified vector supernatants were titrated by transduction of adherent HEK293T cells followed by flow cytometry [A] or integration assay [B].

FIG. 38: Enhancement of lentiviral vector titres by modified U1 snRNA appears to be independent of reported ability of ‘AU’ dinucleotide dependency of U1 snRNA in generating splicing commitment complexes. HIV-EF1a-GFP vector was produced in suspension (serum-free) HEK293T cells with the stated dinucleotide variants of 256U1 snRNA (see Table V), and titrated on adherent HEK293T cells. Relative titres (to no 256U1) were plotted and ranked from highest to lowest impact on titres (13nt variants; light grey bars), as well as predicted CBP20 binding scores, according to Yeh et al (2017). The dotted line indicates the titre increase by the 256U1_13_aT control variant. There appeared to be no correlation between the predicted ability of each dinucleotide variants' CAP binding score, and the enhancement in vector titre.

FIG. 39: Fine-tuning of a modified U1 targeting site. Modified U1 snRNAs containing 13 nucleotide target-annealing lengths were designed (Table VI) based on the apparent ‘hotspot’ identified by the 256U1 snRNA (see FIGS. 4 & 5). These were designed so that the target site was shifted up or downstream of the nt256 target site in approximately 2 nt increments in the HIV-1 based LV genome. HIV-EF1a-GFP vectors were produced in suspension (serum-free) HEK293T cells by transient transfection in the absence or presence of each of the denoted variant modified U1 snRNAs. Clarified vector supernatants were titrated by transduction of adherent HEK293T cells, followed by analysis by flow cytometry.

FIG. 40: Enhancement of EIAV-based lentiviral vector titres by co-transfection of production cells with modified U1 snRNAs directed to the 5′packaging region of the EIAV vRNA. Suspension (serum-free) HEK293T cells were transfected with either pEIAV-CMV-GFP or pEIAV-EF1a-GFP genome plasmids, and pGagPol, pRev and pVSVG, and with or without the indicated modified U1 snRNA expression plasmids (see Table VI). Clarified vector supernatants were titrated by transduction of adherent HEK293T cells followed by flow cytometry. Relative titres were plotted compared to vectors produced in the absence of modified U1 snRNA expression plasmid (striped bars).

FIG. 41: Enhancement of SIVagm-based lentiviral vector titres by co-transfection of production cells with modified U1 snRNAs directed to the 5′packaging region of the SIVagm vRNA. Suspension (serum-free) HEK293T cells were transfected with SIV vector components, and with or without the indicated modified U1 snRNA expression plasmids (see Table VIII). Clarified vector supernatants were titrated by transduction of adherent HEK293T cells followed by integration assay.

### DETAILED DESCRIPTION OF THE INVENTION

### General Definitions

The practice of the present invention will employ, unless otherwise indicated, conventional techniques of chemistry, molecular biology, microbiology and immunology, which are within the capabilities of a person of ordinary skill in the art. Such techniques are explained in the literature. See, for example, J. Sambrook, E. F. Fritsch, and T. Maniatis (1989) Molecular Cloning: A Laboratory Manual, Second Edition, Books 1-3, Cold Spring Harbor Laboratory Press; Ausubel, F. M. et al. (1995 and periodic supplements) Current Protocols in Molecular Biology, Ch. 9, 13, and 16, John Wiley & Sons, New York, N.Y.; B. Roe, J. Crabtree, and A. Kahn (1996) DNA Isolation and Sequencing: Essential Techniques, John Wiley & Sons; J. M. Polak and James O′D. McGee (1990) In Situ Hybridization: Principles and Practice; Oxford University Press; M. J. Gait (ed.) (1984) Oligonucleotide Synthesis: A Practical Approach, IRL Press; and, D. M. J. Lilley and J. E. Dahlberg (1992) Methods of Enzymology: DNA Structure Part A: Synthesis and Physical Analysis of DNA Methods in Enzymology, Academic Press. Each of these general texts is herein incorporated by reference.

Unless defined otherwise, all technical and scientific terms used herein have the same meaning as is commonly understood by one of skill in the art to which this invention belongs. Unless otherwise specified, “rev” and “gag-pol” refer to the proteins and/or genes of lentiviral vectors.

The term “protein”, as used herein, includes proteins, polypeptides, and peptides. As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

As used herein, the term “nucleotide sequence” is synonymous with the term “polynucleotide” and/or the term “nucleic acid sequence”.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Modified U1 snRNA

The present inventors have surprisingly found that the output titres of lentiviral vectors can be enhanced by co-expressing non-coding RNAs based on U1 snRNAs, which have been modified so that they no longer target the endogenous sequence (a splice donor site) but now target a sequence within the vRNA molecule. The present invention relates to such modified U1 snRNAs and a novel method to increase the production titres of lentiviral vectors. The approach consists of the co-expression of modified U1 snRNAs together with the other vector components during vector production. The modified U1 snRNAs are designed such that binding to the consensus splice donor site has been ablated by replacing it with a heterologous sequence that is complementary to a target sequence within the vector genome vRNA. The invention describes various modes of application and optimal characteristics of the modified U1 snRNAs, including target sequence and complementarity length, design and modes of expression.

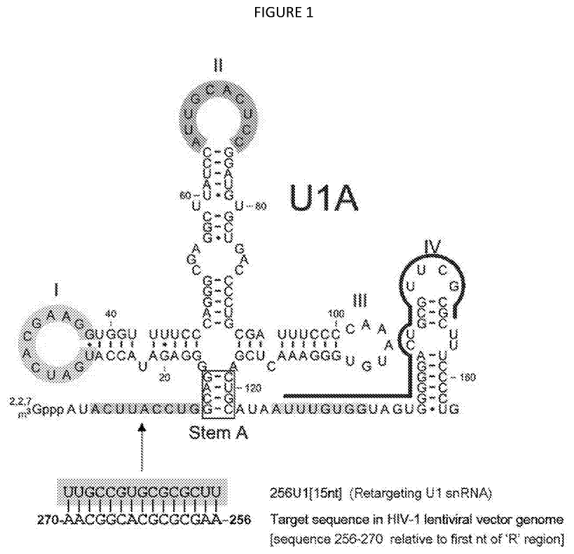

Human U1 snRNA (small nuclear RNA) is 164 nt long with a well-defined structure consisting of four stem—loops (see FIG. 1). The endogenous non-coding RNA, U1 snRNA, binds to the consensus 5′ splice donor site (e.g. 5′-MAGGURR-3′ wherein M is A or C and R is A or G) via the native splice donor annealing sequence (e.g. 5′-ACUUACCUG-3′) during early steps of intron splicing. Stem loop I binds to U1A-70K protein that has been shown to be important for polyA suppression. Stem loop II binds to U1A protein, and the 5′-AUUUGUGG-3′ sequence binds to Sm proteins, which together with Stem loop IV, is important for U1 snRNA processing. In the invention, the modified U1 snRNA is modified to introduce a heterologous sequence that is complementary to a target sequence within the vector genome vRNA molecule at the site of the native splice donor targeting sequence (see FIG. 1).

As used herein, the terms “modified U1 snRNA”, “re-directed U1 snRNA”, “re-targeted U1 snRNA”, “re-purposed U1 snRNA” and “mutant U1 snRNA”, mean a U1 snRNA that has been modified so that it no longer binds the consensus 5′ splice donor site sequence (e.g. 5′-MAGGURR-3′) that it uses to initiate the splicing process of a target gene. Thus, a modified U1 snRNA is a U1 snRNA which has been modified so that it no longer binds to the splice donor site sequence (e.g. 5′-MAGGURR-3′) based on complementarity of the donor site sequence with the native splice donor annealing sequence at the 5′ end of the U1 snRNA. Instead, the modified U1 snRNA is designed so that it binds a nucleotide sequence having a unique RNA sequence within the packaging region of the lentiviral vector genome molecule (target site), i.e. a sequence that is unrelated to splicing of the gene. The nucleotide sequence within the packaging region of the lentiviral vector genome molecule can be preselected. Thus, the modified U1 snRNA is a U1 snRNA which has been modified so that its 5′ end binds a nucleotide sequence within the packaging region of the lentiviral vector genome molecule. As a result, the modified U1 snRNA binds to the target site sequence based on complementarity of the target site sequence with the short sequence at the 5′ end of the modified U1 snRNA.

The 5′ packaging region of the lentiviral vector may have a sequence known in the art. For example, the 5′ packaging region of the lentiviral vector may be any one of the following:

HIV-1 (HxB2) [GenBank: K03455.1]:
SEQ ID NO: 67
gggtctctctggttagaccagatctgagcctgggagctctctggctaactagggaacccactgcttaagcctcaataaagcttg
ccttgagtgcttcaagtagtgtgtgcccgtctgttgtgtgactctggtaactagagatccctcagacccttttagtcagtgtgg
aaaatctctagcagtggcgcccgaacagggacctgaaagcgaaagggaaaccagaggagctctctcgacgcaggactcggcttg
ctgaagcgcgcacggcaagaggcgaggggcggcgactggtgagtacgccaaaaattttgactagcggaggctagaaggagagag
atgggtgcgagagcgtcagtattaagcgggggagaattagatcgatgggaaaaaattcggttaaggccagggggaaagaaaaaa
tataaattaaaacatatagtatgggcaagcagggagctagaacgattcgcagttaatcctggcctgttagaaacatcagaaggc
tgtagacaaatactgggacagctacaaccatcccttcagacaggatcagaagaacttagatcattatataatacagtagcaacc
ctctattgtgtgcatcaaaggatagagataaaagacaccaaggaagctttagacaagatagaggaagagcaaaacaaaagtaag
a
HIV-2 [NCBI Reference Sequence: NC_001722.1]:
SEQ ID NO: 68
cagtcgctctgcggagaggctggcagatcgagccctgagaggttctctccagcactagcaggtagagcctgggtgttccctgct
ggactctcaccagtacttggccggtactgggcagacggctccacgcttgcttgcttaaagacctcttcaataaagctgccagtt
agaagcaagttaagtgtgtgttcccatctctcctagtcgccgcctggtcattcggtgttcatctgagtaacaagaccctggtct
gttaggacccttctcgctttgggaatccaaggcaggaaaatccctagcaggttggcgcccgaacagggacttgaagaggactga
gaagccctggaactcggctgagtgaaggcagtaagggcggcaggaacaaaccacgacggagtgctcctagaaaggcgcgggccg
aggtaccaaaggcggcgtgtggagcgggagtgaaagaggcctccgggtgaaggtaagtacctacaccaaaaactgtagccagaa
aaggcttgttatcctacctttagacaggtagaagattgtgggagatgggcgcgagaaactccgtcttgagagggaaaaaagcag
acgaattagaaaaagttaggttacggcccggcggaaagaaaaagtacaggttaaaacatattgtgtgggcagcgaatgaattgg
ataaattcggattggcagagagcctgttggagtcaaaagaaggttgccaaaagattctcagagttttagatccattagtaccaa
cagggtcagaaaatttaaaaagcctttttaataccgtctgcgtcatttggtgcttgcacgcagaagagaaagtgaaagatactg
aggaagcaaagaaactagcacagagacatctagtggcagaaactggaactgcagagaaaatgccaaatacaagtagaccaacag
caccacctagtgggaaaagaggaaactaccccgtgcaacaagcgggtggcaactatgtccatgtgccactga
EIAV (SPEIAV-19 strain) [GenBank: U01866.1]:
SEQ ID NO: 69
gggactcagattctgcggtctgagtcccttctctgctgggctgaaaaggcctttgtaataaatataattctctactcagtccct
gtctctagtttgtctgttcgagatcctacagttggcgcccgaacagggacctgagaggggcgcagaccctacctgttgaacctg
gctgatcgtaggatccccgggacagcagaggagaacttacagaagtcttctggaggtgttcctggccagaacacaggaggacag
gtaagatgggagaccctttgacatggagcaaggcgctcaagaagttagagaaggtgacggtacaagggtctcagaaattaacta
ctggtaactgtaattgggcgctaagtctagtagacttatttcatgataccaactttgtaaaagaaaaggactggcagctgaggg
atgtcattccattgctggaagatgtaactcagacgctgtcaggacaagaaagagaggcctttgaaagaacatggtgggcaattt
ctgctgtaaagatgggcctccagattaataatgtagtagatggaaaggcatcattccagctcctaagagcgaaatatgaaaaga
agactgctaataaaaagcagtctgagccctctgaagaatatc
SIVagm (TYO-1 strain) [GenBank: AB253736.1]:
SEQ ID NO: 70
cagtctcttactaggagaccagcttgagcctgggtgttcgctggttagcctaacctggttggccaccaggggtaaggactcctt
ggcttagaaagctaataaacttgcctgcattagagcttatctgagtcaagtgtcctcattgacgcctcactctcttgaacggga
atcttccttactgggttctctctctgacccaggcgagagaaactccagcagtggcgcccgaacagggacttgagtgagagtgta
ggcacgtacagctgagaaggcgtcggacgcgaaggaagcgcggggtgcgacgcgaccaagaaggagacttggtgagtaggcttc
tcgagtgccgggaaaaagctcgagcctagttagaggactaggagaggccgtagccgtaactactctgggcaagtagggcaggcg
gtgggtacgcaatgggggcggctacctcagcactaaataggagacaattagaccaatttgagaaaatacgacttcgcccgaacg
gaaagaaaaagtaccaaattaaacatttaatatgggcaggcaaggagatggagcgcttcggcctccatgagaggttgttggaga
cagaggaggggtgtaaaagaatcatagaagtcctctaccccctagaaccaacaggatcggagggcttaaaaagtctgttcaatc
ttgtgtgcgtactatattgcttgcacaaggaacagaaagtgaaagacacagaggaagcagtagcaacagtaagacaacactgcc
atctagtggaaaaagaaaaaagtgc
SIVmac (Mm251 strain) [GenBank: M19499.1]:
SEQ ID NO: 71
cagtcgctctgcggagaggctggcagattgagccctgggaggttctctccagcactagcaggtagagcctgggtgttccctgct
agactctcaccagcacttggccagtgctgggcagagtggctccacgcttgcttgcttaaagacctcttcaataaagctgccatt
ttagaagtaagccagtgtgtgttcccatctctcctagtcgccgcctggtcaactcggtactcggtaataagaagaccctggtct
gttaggaccctttctgctttgagaaaccgaagcaggaaaatccctagcagattggcgcccgaacaggacttgaaggagagtgag
agactcctgagtacggctgagtgaaggcagtaagggcggcaggaaccaaccacgacggagtgctcctataaaggcgcgggtcgg
taccagacggcgtgaggagcgggagaggaggaggcctccggttgcaggtaagtgcaacacaaaaaagaaatagctgtcttgtta
tccaggaagggataataagatagagtgggagatgggcgcgagaaactccgtcttgtcagggaagaaagcagatgaattagaaaa
aattaggctacgacccggcggaaagaaaaagtacatgttgaagcatgtagtatgggcagcaaatgaattagatagatttggatt
agcagaaagcctgttggagaacaaagaaggatgtcaaaaaatactttcggtcttagctccattagtgccaacaggctcagaaaa
tttaaaaagcctttataatactgtctgcgtcatctggtgcattcacgcagaagagaaagtgaaacacactgaggaagcaaaaca
gatagtgcagagacacctagtggtg
FIV (Petaluma strain, clone: 34TF10) [GenBank: M25381.1]:
SEQ ID NO: 72
gagtctctttgttgaggacttttgagttctcccttgaggctcccacagatacaataaatatttgagattgaaccctgtcgagta
tctgtgtaatcttttttacctgtgaggtctcggaatccgggccgagaacttcgcagttggcgcccgaacagggacttgattgag
agtgattgaggaagtgaagctagagcaatagaaagctgttaagcagaactcctgctgacctaaatagggaagcagtagcagacg
ctgctaacagtgagtatctctagtgaagcggactcgagctcataatcaagtcattgtttaaaggcccagataaattacatctgg
tgactcttcgcggaccttcaagccaggagattcgccgagggacagtcaacaaggtaggagagattctacagcaacatggggaat
ggacaggggcgagattggaaaatggccattaagagatgtagtaatgttgctgtaggagtaggggggaagagtaaaaaatttgga
gaagggaatttcagatgggccattagaatggctaatgtatctacaggacgagaacctggtgatataccagagactttagatcaa
ctaaggttggttatttgcgatttacaagaaagaagagaaaaatttggatctagcaaagaaattgatatggcaattgtgacatta
aaagtctttgcggtagcaggacttttaaatatgacggtgtctactgctgctgcagctgaaaatatgtattctcaaatgggatta
gacactag

A suitable modified U1 snRNA may be designed to bind a packaging region of a particular vector type. For example, the use of modified U1 snRNA to increase the output titres of lentiviral vectors can be achieved by observing the following general procedure:
1. The lentiviral vector genome sequence should be obtained, and a set of target sequences within the 5′ packaging sequence region of the vector genome RNA molecule identified. The broad 5′ packaging sequence region is from the first nucleotide of the vector genome RNA molecule to the 3′ nucleotide of the remaining wild type gag sequence that is typically retained as part of the packaging sequence.
2. It is recommended that a panel of target sequences of 15 nucleotides in length be identified for the initial target screen, and that 15-20 different (non-overlapping) sequences be identified first. These should be equally distributed over the packaging sequence from the first nucleotide of the vRNA to the ˜50th nucleotide of the gag region, with fewer sequences identified within the retained gag region.
3. These target sequences present within the vector genome RNA (5′-3′) should be reverse-complemented to give the 15 nucleotide target-annealing sequence that will be encoded within the first nucleotides of the modified U1 snRNA molecule (5′-3′).
4. The target-annealing sequence will be inserted into a U1 snRNA expression cassette (containing the U1 promoter and termination region), replacing native U1 snRNA nucleotides 3-to-11, i.e. the ‘AT’ dinucleotide (nucleotides 1 and 2 of native U1 snRNA [′AU′ in the snRNA molecule]) should be retained upstream of the target-annealing sequence, with the ‘A’ being the transcription start site. This can be achieved by standard molecular cloning/gene synthesis techniques.
5. The panel of modified U1 snRNA expression constructs should then be screened by production of the lentiviral vector encoding the transgenic sequence of interest, wherein each modified U1 snRNA expression construct is individually expressed with vector components. This is most easily performed by transient co-transfection with vector components but could also be performed in a packaging or producer cell line. The output vector supernatant is then titrated to empirically determine the primary target region(s) that results in the greatest titre increase.
6. The modified U1 snRNA may be further improved by generating variants that contain target-annealing sequences that incrementally target the vector genome RNA upstream and downstream of the initial, empirically identified target site. The incremental scanning can be achieved by moving the target-annealing sequence stepwise by every one, two, three or four or more nucleotides per variant, up or downstream of the of the initial, empirically identified target site, possibly continuing to a target position tested in a previous (initial) screen. This may identify the optimal target site within the vector genome RNA
7. The modified U1 snRNA may be further improved by generating variants containing target-annealing sequences that vary in length. It is recommended to take a modified U1 snRNA identified from a previous screen (this may have a target-annealing sequence of 15 nucleotides) and design variants in which the target-annealing sequence is reduced or increased incrementally so that a new panel of variants is produced, wherein the target-annealing sequence of each variant may be 9, 10, 11, 12, 13, 14, 15, 16, 17, 18, 19, 20, 21, 22, 23, 24, 25 or more nucleotides in length. This new panel of modified U1 snRNA variants can then be screened as before.
8. The modified U1 snRNA may be further improved by generating variants containing alternative nucleotides at position 1 and 2. Specifically, the ‘AT’ dinucleotide (‘AU’ within the snRNA molecule) may be altered away from this consensus, for example to ‘GA’. This new panel of modified U1 snRNA variant can then be screened as before.
9. The modified U1 snRNA expression construct may be encoded within a DNA molecule (e.g. plasmid DNA) separate to lentiviral vector components or may be operably linked to DNA encoding the vector genome or gagpol or other DNA components co-transfected with the vector components (e.g. a TRAP-expressing plasmid).
10. The modified U1 snRNA expression construct may be stably transfected to generate a cell line, which may be used to generate lentiviral vectors by transient transfection. For example, the cell line may also be stably transfected with a transcriptional repressor such as tetR and/or a translational repressor such as TRAP, or both of these types of expression control proteins.
11. The modified U1 snRNA expression construct may be stably transfected with other lentiviral vector components to generate a packaging or producer cell line
12. An alternative way of obtaining stable cell lines as described above is to insert the modified U1 snRNA expression cassette within a self-inactivating retroviral or lentiviral vector, produce vector virions encoding said modified U1 snRNA expression cassette, and transduce cells at a controlled multiplicity of infection (MOI) in order to isolate stable cell lines more likely to contain a target copy-number of said modified U1 snRNA expression cassette. Said self-inactivating retroviral or lentiviral vector may also contain a selection marker cassette.
13. In order to evaluate RNA or DNA copy-numbers of the modified U1 snRNA sequence within lentiviral vector production cells or lentiviral vector product, an RT-qPCR assay method can be developed in which the forward primer anneals to the specific target-annealing sequence of the modified U1 snRNA in order to unambiguously detect modified U1 snRNA over endogenous/native U1 snRNA.

As used herein, the terms “native splice donor annealing sequence” and “native splice donor targeting sequence” mean the short sequence at the 5′-end of the endogenous U1 snRNA that is broadly complementary to the consensus 5′ splice donor site of introns. The native splice donor annealing sequence may be 5′-ACUUACCUG-3′.

As used herein, the term “consensus 5′ splice donor site” means the consensus RNA sequence at the 5′ end of introns used in splice-site selection, e.g. having the sequence 5′-MAGGURR-3′.

As used herein, the terms “nucleotide sequence within the packaging region of the lentiviral vector genome sequence”, “target sequence” and “target site” mean a site having a particular RNA sequence within the packaging region of the lentiviral vector genome molecule which has been preselected as the target site for binding the modified U1 snRNA.

As used herein, the terms “packaging region of a lentiviral vector genome molecule” and “packaging region of a lentiviral vector genome sequence” means the region at the 5′ end of a lentiviral vector genome from the beginning of the 5′ U5 domain to the terminus of the sequence derived from gag gene. Thus, the packaging region of a lentiviral vector genome molecule includes the 5′ U5 domain, PBS element, stem loop (SL) 1 element, SL2 element, SL3ψ element, SL4 element and the sequence derived from the gag gene. It is common in the art to provide the complete gag gene in trans to the genome during lentiviral vector production to enable the production of replication-defective viral vector particle. The nucleotide sequence of the gag gene provided in trans need not be encoded by wild type nucleotides but may be codon-optimised; importantly the chief attribute of the gag gene provided in trans is that it encodes and directs expression of the gag and gagpol proteins. Accordingly, it will be understood by the person skilled in the art that, if the complete gag gene is to be provided in trans during lentiviral vector production, the term “packaging region of a lentiviral vector genome molecule” may mean the region at the 5′ end of the lentiviral vector genome molecule from the beginning of the 5′ U5 domain through to the ‘core’ packaging signal at the SL3 ψ element, and the native gag nucleotide sequence from the ATG codon (present within SL4) to the end of the remaining gag nucleotide sequence present on the vector genome.

As used herein, the term “sequence derived from gag gene” means, any native sequence of the gag gene derived from the ATG codon to nucleotide 688 (Kharytonchyk, S. et. al., 2018, J. Mol. Biol., 430:2066-79) that may be present, e.g. remain, in the vector genome.

As used herein, the terms “to introduce within the first 11 nucleotides of the U1 snRNA, which encompasses the native splice donor annealing sequence, a heterologous sequence”, “to introduce within the nine nucleotides at positions 3-to-11 said heterologous sequence” and “to introduce within the first 11 nucleotides at the 5′ end of the U1 snRNA a heterologous sequence” include to replace the first 11 nucleotides, or the nine nucleotides at positons 3-to-11, of the U1 snRNA all or in part with said heterologous sequence or to modify the first 11 nucleotides, or the nine nucleotides at positons 3-to-11, of the U1 snRNA to have the same sequence as said heterologous sequence.

As used herein, the terms “to introduce within the native splice donor annealing sequence a heterologous sequence” and “to introduce within the native splice donor annealing sequence at the 5′ end of the U1 snRNA a heterologous sequence” include to replace the native splice donor annealing sequence all or in part with said heterologous sequence or to modify the native splice donor annealing sequence to have the same sequence as said heterologous sequence.

As used herein, the term “enhances lentiviral vector titres” includes “increases lentiviral vector titres” and “improves lentiviral vector titres”.

Accordingly, in one aspect, the present invention provides a modified U1 snRNA which has been modified to bind to a nucleotide sequence within the packaging region of a lentiviral vector genome sequence.

In some embodiments, the modified U1 snRNA is modified at the 5′ end relative to the endogenous U1 snRNA to introduce a heterologous sequence that is complementary to a nucleotide sequence within the packaging region of a lentiviral vector genome sequence.

In some embodiments, the modified U1 snRNA is modified at the 5′ end relative to the endogenous U1 snRNA to introduce within the native splice donor annealing sequence a heterologous sequence that is complementary to a nucleotide sequence within the packaging region of a lentiviral vector genome sequence.

The modified U1 snRNA may be modified at the 5′ end relative to the endogenous U1 snRNA to replace a sequence encompassing the native splice donor annealing sequence with a heterologous sequence that is complementary to said nucleotide sequence.

The modified U1 snRNA may be a modified U1 snRNA variant. The U1 snRNA variant which is modified in accordance with the invention may be a naturally occurring U1 snRNA variant, a U1 snRNA variant containing a mutation within the stem loop I region ablating U1-70K protein binding, or a U1 snRNA variant containing a mutation in the stem loop II region ablating U1A protein binding. The U1 snRNA variant containing a mutation within the stem loop I region ablating U1-70K protein binding may be U1_m1 or U1_m2, preferably U1A_m1 or U1A_m2.

In some embodiments, the modified U1 snRNA of the invention comprises a nucleotide sequence having at least 70% identity (suitable at least 75%, at least 80%, 85%, 90%, 91%, 92%, 93%, 94%, 95%, 96%, 97%, 98%, 99% identity) with the main U1 snRNA sequence [clover leaf] (nt 410-562) of the U1_256 sequence as described herein. In some embodiments, the modified U1 snRNA of the invention comprises the main U1 snRNA sequence [clover leaf] (nt 410-562) of the U1_256 sequence as described herein. The main U1 snRNA sequence [clover leaf] (nt 410-562) of the U1_256 sequence (SEQ ID NO:15) is as follows:

(SEQ ID NO: 66)
GCAGGGGAGATACCATGATCACGAAGGTGGTTTTCCCAGGGCGAGGCTTA
TCCATTGCACTCCGGATGTGCTGACCCCTGCGATTTCCCCAAATGTGGGA
AACTCGACTGCATAATTTGTGGTAGTGGGGGACTGCGTTCGCGCTTTCCC
CTG.

In some embodiments, the modified U1 snRNA of the invention comprises a nucleotide sequence as follows:

(N[0-2])XGCAGGGGAGATACCATGATCACGAAGGTGGTTTTCCCAGGG
CGAGGCTTATCCATTGCACTCCGGATGTGCTGACCCCTGCGATTTCCCCA
AATGTGGGAAACTCGACTGCATAATTTGTGGTAGTGGGGGACTGCGTTCG
CGCTTTCCCCTG (where X is the target-annealing
sequence of undefined length; N is any of A, C G
or T; N[0-2] is a nucleotide sequence of 0-2
nucleotides in length; and the main U1 snRNA
sequence [cloverleaf] (SEQ ID NO: 66) is shown
underlined).

In some embodiments, the modified U1 snRNA of the invention comprises a nucleotide sequence as follows:

ATXGCAGGGGAGATACCATGATCACGAAGGTGGTTTTCCCAGGGCGAGGC
TTATCCATTGCACTCCGGATGTGCTGACCCCTGCGATTTCCCCAAATGTG
GGAAACTCGACTGCATAATTTGTGGTAGTGGGGGACTGCGTTCGCGCTTT
CCCCTG (where X is the target-annealing sequence
of undefined length and the main U1 snRNA sequence
[clover leaf] (SEQ ID NO: 66) is shown underlined)

In some embodiments, the modified U1 snRNA of the invention comprises a nucleotide sequence as follows:

AT(N[9-15])GCAGGGGAGATACCATGATCACGAAGGTGGTTTTCCCAG
GGCGAGGCTTATCCATTGCACTCCGGATGTGCTGACCCCTGCGATTTCCC
CAAATGTGGGAAACTCGACTGCATAATTTGTGGTAGTGGGGGACTGCGTT
CGCGCTTTCCCCTG (where N is any of A, C, G or T;
N[9-15] is the target annealing sequence of 9-15
nucleotides in length; and the main U1 snRNA
sequence [cloverleaf] (SEQ ID NO: 66) is shown
underlined).

In some preferred embodiments, the first 11 nucleotides of the U1 snRNA, which encompasses the native splice donor annealing sequence, may be all or in part replaced with a heterologous sequence that is complementary to a nucleotide sequence within the packaging region of a lentiviral vector genome sequence. Suitably, 1-11 (suitably 2-11, 3-11, 1, 2, 3, 4, 5, 6, 7, 8, 9, 10 or 11), nucleic acids of the first 11 nucleotides of the U1 snRNA are replaced with a heterologous sequence that is complementary to a nucleotide sequence within the packaging region of a lentiviral vector genome sequence.

In some embodiments, the native splice donor annealing sequence, may be all or in part replaced with a heterologous sequence that is complementary to a nucleotide sequence within the packaging region of a lentiviral vector genome sequence. Suitably, 1-11 (suitably 2-11, 3-11, 5-11, 1, 2, 3, 4, 5, 6, 7, 8, 9, 10 or 11), nucleic acids of the native splice donor annealing sequence are replaced with a heterologous sequence that is complementary to a nucleotide sequence within the packaging region of a lentiviral vector genome sequence. In a preferred embodiment, the entire native splice donor annealing sequence is replaced with a heterologous sequence that is complementary to a nucleotide sequence within the packaging region of a lentiviral vector genome sequence, i.e. the native splice donor annealing sequence (e.g. 5′-ACUUACCUG-3′) is fully replaced with a heterologous sequence in accordance with the invention.

In some embodiments, the modified U1 snRNA comprising a heterologous sequence that is complementary to a nucleotide sequence within the packaging region of a lentiviral vector genome sequence will encode an A at the first nucleotide at the 5′ end of said heterologous sequence, irrespective of whether the A partakes in annealing to the target sequence.

In some embodiments, the modified U1 snRNA comprising a heterologous sequence that is complementary to a nucleotide sequence within the packaging region of a lentiviral vector genome sequence will encode an AU at the first two nucleotides at the 5′ end of said heterologous sequence, irrespective of whether the A or the U partakes in annealing to the target sequence.

In some embodiments, the modified U1 snRNA comprising a heterologous sequence that is complementary to a nucleotide sequence within the packaging region of a lentiviral vector genome sequence will not encode AU at the first two nucleotides at the 5′ end of said heterologous sequence, and the first nucleotide may or may not partake in annealing to the target sequence.

In some embodiments, a heterologous sequence that is complementary to a nucleotide sequence within the packaging region of a lentiviral vector genome sequence comprises at least 7 nucleotides of complementarity to said nucleotide sequence. In some embodiments, a heterologous sequence that is complementary to a nucleotide sequence within the packaging region of a lentiviral vector genome sequence comprises at least 9 nucleotides of complementarity to said nucleotide sequence. Preferably, a heterologous sequence for use in the present invention comprises 15 nucleotides of complementarity to said nucleotide sequence.

Suitably, a heterologous sequence for use in the present invention may comprise 7-25 (suitably 7-20, 7-15, 9-15, 7, 8, 9, 10, 11, 12, 13, 14, 15, 16, 17, 18, 19, 20, 21, 22, 23, 24 or 25) nucleotides.

Suitably, a heterologous sequence for use in the present invention may comprise 7 nucleotides.

Suitably, a heterologous sequence for use in the present invention may comprise 8 nucleotides.

Suitably, a heterologous sequence for use in the present invention may comprise 9 nucleotides.

Suitably, a heterologous sequence for use in the present invention may comprise 10 nucleotides.

Suitably, a heterologous sequence for use in the present invention may comprise 11 nucleotides.

Suitably, a heterologous sequence for use in the present invention may comprise 12 nucleotides.

Suitably, a heterologous sequence for use in the present invention may comprise 13 nucleotides.

Suitably, a heterologous sequence for use in the present invention may comprise 14 nucleotides.

Suitably, a heterologous sequence for use in the present invention may comprise 15 nucleotides.

Suitably, a heterologous sequence for use in the present invention may comprise 16 nucleotides.

Suitably, a heterologous sequence for use in the present invention may comprise 17 nucleotides.

Suitably, a heterologous sequence for use in the present invention may comprise 18 nucleotides.

Suitably, a heterologous sequence for use in the present invention may comprise 19 nucleotides.

Suitably, a heterologous sequence for use in the present invention may comprise 20 nucleotides.

Suitably, a heterologous sequence for use in the present invention may comprise 21 nucleotides.

Suitably, a heterologous sequence for use in the present invention may comprise 22 nucleotides.

Suitably, a heterologous sequence for use in the present invention may comprise 23 nucleotides.

Suitably, a heterologous sequence for use in the present invention may comprise 24 nucleotides.

Suitably, a heterologous sequence for use in the present invention may comprise 25 nucleotides.

In some embodiments, the nucleotide sequence within the packaging region of a lentiviral vector genome sequence is located within the 5′ U5 domain, PBS element, SL1 element, SL2 element, SL3ψ element, SL4 element and/or the sequence derived from gag gene. Suitably, the nucleotide sequence within the packaging region of a lentiviral vector genome sequence is located within the SL1, SL2 and/or SL3ψ element(s). In some preferred embodiments, the nucleotide sequence within the packaging region of a lentiviral vector genome sequence is located within the SL1 and/or SL2 element(s). In some particularly preferred embodiments, the nucleotide sequence within the packaging region of a lentiviral vector genome sequence is located within the SL1 element.

In some embodiments, a nucleotide sequence within the packaging region of a lentiviral vector genome sequence comprises at least 7 nucleotides. In some embodiments, a nucleotide sequence within the packaging region of a lentiviral vector genome sequence comprises at least 9 nucleotides. Suitably, a nucleotide sequence within the packaging region of a lentiviral vector genome sequence comprises 7-25 (suitably 7-20, 7-15, 9-15, 7, 8, 9, 10, 11, 12, 13, 14, or 15) nucleotides.

Suitably, a nucleotide sequence within the packaging region of a lentiviral vector genome sequence comprises 7 nucleotides.

Suitably, a nucleotide sequence within the packaging region of a lentiviral vector genome sequence comprises 8 nucleotides.

Suitably, a nucleotide sequence within the packaging region of a lentiviral vector genome sequence comprises 9 nucleotides.

Suitably, a nucleotide sequence within the packaging region of a lentiviral vector genome sequence comprises 10 nucleotides.

Suitably, a nucleotide sequence within the packaging region of a lentiviral vector genome sequence comprises 11 nucleotides.

Suitably, a nucleotide sequence within the packaging region of a lentiviral vector genome sequence comprises 12 nucleotides.

Suitably, a nucleotide sequence within the packaging region of a lentiviral vector genome sequence comprises 13 nucleotides.

Suitably, a nucleotide sequence within the packaging region of a lentiviral vector genome sequence comprises 14 nucleotides.

Suitably, a nucleotide sequence within the packaging region of a lentiviral vector genome sequence comprises 15 nucleotides.

Suitably, a nucleotide sequence within the packaging region of a lentiviral vector genome sequence comprises 16 nucleotides.

Suitably, a nucleotide sequence within the packaging region of a lentiviral vector genome sequence comprises 17 nucleotides.

Suitably, a nucleotide sequence within the packaging region of a lentiviral vector genome sequence comprises 18 nucleotides.

Suitably, a nucleotide sequence within the packaging region of a lentiviral vector genome sequence comprises 19 nucleotides.

Suitably, a nucleotide sequence within the packaging region of a lentiviral vector genome sequence comprises 20 nucleotides.

Suitably, a nucleotide sequence within the packaging region of a lentiviral vector genome sequence comprises 21 nucleotides.

Suitably, a nucleotide sequence within the packaging region of a lentiviral vector genome sequence comprises 22 nucleotides.

Suitably, a nucleotide sequence within the packaging region of a lentiviral vector genome sequence comprises 23 nucleotides.

Suitably, a nucleotide sequence within the packaging region of a lentiviral vector genome sequence comprises 24 nucleotides.

Suitably, a nucleotide sequence within the packaging region of a lentiviral vector genome sequence comprises 25 nucleotides.

Preferably, a nucleotide sequence within the packaging region of a lentiviral vector genome sequence comprises 15 nucleotides.

The binding of a modified U1 snRNA of the invention to the nucleotide sequence within the packaging region of a lentiviral vector genome sequence may enhance lentiviral vector titre during lentiviral vector production relative to lentiviral vector production in the absence of a modified U1 snRNA of the invention. Thus, production of a lentiviral vector in the presence of a modified U1 snRNA of the invention enhances lentiviral vector titre relative to lentiviral vector production in the absence of a modified U1 snRNA of the invention. A suitable assay for the measurement of lentiviral vector titre is as described herein. Suitably, the lentiviral vector production involves co-expression of said modified U1 snRNA with vector components including gag, env, rev and the RNA genome of the lentiviral vector. In some embodiments, the enhancement of lentiviral vector titre occurs in the presence or absence of a functional 5′LTR polyA site. In some embodiments, the enhancement of lentiviral vector titres mediated by a modified U1 snRNA of the invention is independent of polyA site suppression in the 5′LTR of the vector genome.

In some embodiments, the binding of a modified U1 snRNA of the invention to the nucleotide sequence within the packaging region of a lentiviral vector genome sequence may increase lentiviral vector titre during lentiviral vector production by at least 30% relative to lentiviral vector production in the absence of a modified U1 snRNA of the invention. Suitably, the binding of a modified U1 snRNA of the invention to the nucleotide sequence within the packaging region of a lentiviral vector genome sequence may increase lentiviral vector titre during lentiviral vector production by at least 35% (suitably at least 40%, 45%, 50%, 60%, 70%, 100%, 150%, 200%, 250%, 300%, 350%, 400%, 450%, 500%, 550%, 600%, 650%, 700%, 750%, 800%, 850%, 900%, 950% or 1000%) relative to lentiviral vector production in the absence of a modified U1 snRNA of the invention.

The modified U1 snRNAs of the present invention may be designed by (a) selecting a target site in the packaging region of a lentiviral vector genome for binding the modified U1 snRNA (the preselected nucleotide site); and (b) introducing within the native splice donor annealing sequence (e.g. 5′-ACUUACCUG-3′) at the 5′ end of the U1 snRNA a heterologous sequence that is complementary to the preselected nucleotide site selected in step (a).

The introduction of a heterologous sequence that is complementary to the target site within, or in place of, the native splice donor annealing sequence (e.g. 5′-ACUUACCUG-3′) at the 5′ end of the endogenous U1 snRNA using conventional techniques in molecular biology is within the capabilities of a person of ordinary skill in the art. Generally speaking, suitable routine methods include directed mutagenesis or replacement via homologous recombination.

The modification of the native splice donor annealing sequence (e.g. 5′-ACUUACCUG-3′) at the 5′ end of the endogenous U1 snRNA to have the same sequence as a heterologous sequence that is complementary to the target site using conventional techniques in molecular biology is within the capabilities of a person of ordinary skill in the art. For example, suitable methods include directed mutagenesis or random mutagenesis followed by selection for mutations which provide a modified U1 snRNA in accordance with the invention.

The modified U1 snRNAs of the present invention can be manufactured according to methods generally known in the art. For example, the modified U1 snRNAs can be manufactured by chemical synthesis or recombinant DNA/RNA technology.

The introduction of a nucleotide sequence encoding a modified U1 snRNA of the present invention into a cell using conventional molecular and cell biology techniques is within the capabilities of a person of ordinary skill in the art. For example, an expression cassette could be used as described below.

Accordingly, in a further aspect, the present invention provides a cell comprising a modified U1 snRNA of the invention. Suitable cells are described below.

In a further aspect, as demonstrated in the present Examples, the modified U1 according to the present invention as described herein may beneficially result in repression of transgene expression. As such, the invention also encompasses a method for transgene repression using modified U1, or methods or uses thereof, as described herein.

### Nucleotide Sequence

In a further aspect, the present invention provides a nucleotide sequence encoding a modified U1 snRNA of the invention.

The term “nucleotide sequence” in relation to the present invention can be a double stranded or single stranded molecule and includes genomic DNA, cDNA, synthetic DNA, RNA and a chimeric DNA/RNA molecule. Preferably it means DNA, more preferably cDNA sequence coding for a modified U1 snRNA of the present invention.

Typically, the nucleotide sequence encompassed by the scope of the present invention is prepared using recombinant DNA techniques (i.e. recombinant DNA), as described herein.

In a preferred embodiment, the nucleotide sequence encoding a modified U1 snRNA of the invention is an expression cassette.

The presence/abundance of modified U1 snRNA molecule can be quantified within vector production cell extracts or vector virions by extraction of total RNA followed by RT-PCR or RT-qPCR (quantitative) using DNA primers. Importantly, the forward primer is designed such that is has complementarity to the targeting sequence of the modified U1 snRNA molecule so that only the modified U1 snRNA is amplified during qPCR and not endogenous U1 snRNA.

In one aspect the present invention provides a vector virion comprising a modified U1 according to the present invention as described herein.

### Vector/Expression Cassette

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into and expressed by a target cell. The vector may facilitate the integration of the nucleotide sequence encoding the modified U1 snRNA of the invention (or a viral vector component) to maintain the nucleotide sequence encoding the modified U1 snRNA of the invention (or the viral vector component) and its expression within the target cell.

The vector may be or may include an expression cassette (also termed an expression construct). Expression cassettes as described herein comprise regions of nucleic acid containing sequences capable of being transcribed. Thus, sequences encoding mRNA, tRNA and rRNA are included within this definition.

The vector may contain one or more selectable marker genes (e.g. a neomycin resistance gene) and/or traceable marker gene(s) (e.g. a gene encoding green fluorescent protein (GFP)). Vectors may be used, for example, to infect and/or transduce a target cell. The vector may further comprise a nucleotide sequence enabling the vector to replicate in the host cell in question.

The term “cassette”—which is synonymous with terms such as “conjugate”, “construct” and “hybrid”—includes a polynucleotide sequence directly or indirectly attached to a promoter. The expression cassettes of the invention comprise a promoter for the expression of the nucleotide sequence encoding the modified U1 snRNA of the invention and optionally a regulator of the nucleotide sequence encoding the modified U1 snRNA of the invention. Expression cassettes for use in the invention comprise a promoter for the expression of the nucleotide sequence encoding a viral vector component and optionally a regulator of the nucleotide sequence encoding the viral vector component. Preferably the cassette comprises at least a polynucleotide sequence operably linked to a promoter.

The expression cassette may be used to replicate the nucleotide sequence encoding the modified U1 snRNA of the invention in a compatible target cell in vitro. Thus, the invention provides a method of making modified U1 snRNAs in vitro by introducing an expression cassette of the invention into a compatible target cell in vitro and growing the target cell under conditions which result in expression of the modified U1 snRNAs. The modified U1 snRNAs may be recovered from the target cell by methods well known in the art. Suitable target cells include mammalian cell lines and other eukaryotic cell lines.

The choice of expression cassette, e.g. plasmid, cosmid, virus or phage vector, will often depend on the host cell into which it is to be introduced. The expression cassette can be a DNA plasmid (supercoiled, nicked or linearised), minicircle DNA (linear or supercoiled), plasmid DNA containing just the regions of interest by removal of the plasmid backbone by restriction enzyme digestion and purification, DNA generated using an enzymatic DNA amplification platform e.g. doggybone DNA (dbDNA™) where the final DNA used is in a closed ligated form or where it has been prepared (e.g. restriction enzyme digestion) to have open cut ends.

Accordingly, in one aspect the present invention provides an expression cassette comprising a nucleotide sequence encoding a modified U1 snRNA of the invention.

In one aspect of the invention the modified U1 expression cassette as described herein may be delivered to a cell as described herein via a lentiviral vector or a retroviral vector.

The introduction of an expression cassette of the invention into a cell using conventional molecular and cell biology techniques is within the capabilities of a person of ordinary skill in the art.

In a further aspect, the present invention provides a cell comprising an expression cassette of the invention. Suitable cells are described below.

### Lentiviral Vector Production Systems and Cells

A lentiviral vector production system comprises a set of nucleotide sequences encoding the components required for production of the lentiviral vector. Accordingly, a vector production system comprises a set of nucleotide sequences which encode the viral vector components necessary to generate lentiviral vector particles.

“Viral vector production system” or “vector production system” or “production system” is to be understood as a system comprising the necessary components for lentiviral vector production.

In one aspect nucleotide sequence may be suitable for use in a lentiviral vector in a tat-independent system for vector production. As described herein, 3rd generation lentiviral vectors are U3-dependent (and employ a heterologous promoter to drive transcription), and the nucleotide sequences according to the present invention may be used in the context of a 3^{rd} generation lentiviral vector. In one aspect of the invention tat is not provided in the lentiviral vector production system, for example tat is not provided in trans. In one aspect the cell or vector or vector production system as described herein does not comprise the tat protein.

In one aspect the invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated, wherein said nucleotide sequence is for use in a tat-independent lentiviral vector.

In one aspect the invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated, wherein said nucleotide sequence is produced in the absence of tat.

In one aspect the invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated, wherein said nucleotide sequence has been transcribed independently of tat.

In one aspect the invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated, wherein said nucleotide sequence is for use in a U3-independent lentiviral vector.

In one aspect the invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated, wherein said nucleotide sequence has been transcribed independently of the U3 promoter.

In one aspect the invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated, wherein said nucleotide sequence has been transcribed by a heterologous promoter.

In one aspect, transcription of the nucleotide sequence as described herein is not dependent on the presence of U3. The nucleotide sequence may be derived from a U3-independent transcription event. The nucleotide sequence may be derived from a heterologous promoter. A nucleotide sequence as described herein may not comprise a native U3 promoter.

In one embodiment, the viral vector production system comprises nucleotide sequences encoding Gag and Gag/Pol proteins, and Env protein and the vector genome sequence. The production system may optionally comprise a nucleotide sequence encoding the Rev protein, or functional substitute thereof.

In one embodiment of the invention at least one transgene component may be inverted or in the reverse orientation.

In one embodiment, at least one transgene component may be inverted or in the reverse orientation relative to the 5′-3′ directionality of the vector genome RNA. Lentiviral vector genomes wherein the transgene cassette is inverted, i.e. the transcription unit is opposed to the promoter driving the vector genome cassette, may be utilised. In addition, there may be instances where one component of the transgene cassette may be in reverse and another in the forward orientation, for example use of bi-directional transgene cassettes, or multiple separate cassettes.

In one embodiment, the viral vector production system comprises modular nucleic acid constructs (modular constructs). A modular construct is a DNA expression construct comprising two or more nucleic acids used in the production of lentiviral vectors. A modular construct can be a DNA plasmid comprising two or more nucleic acids used in the production of lentiviral vectors. The plasmid may be a bacterial plasmid. The nucleic acids can encode for example, gag-pol, rev, env, vector genome. In addition, modular constructs designed for generation of packaging and producer cell lines may additionally need to encode transcriptional regulatory proteins (e.g. TetR, CymR) and/or translational repression proteins (e.g. TRAP) and selectable markers (e.g Zeocin™, hygromycin, blasticidin, puromycin, neomycin resistance genes). Suitable modular constructs for use in the present invention are described in EP 3502260, which is hereby incorporated by reference in its entirety.

As the modular constructs for use in accordance with the present invention contain nucleic acid sequences encoding two or more of the retroviral components on one construct, the safety profile of these modular constructs has been considered and additional safety features directly engineered into the constructs. These features include the use of insulators for multiple open reading frames of retroviral vector components and/or the specific orientation and arrangement of the retroviral genes in the modular constructs. It is believed that by using these features the direct read-through to generate replication-competent viral particles will be prevented.

The nucleic acid sequences encoding the viral vector components may be in reverse and/or alternating transcriptional orientations in the modular construct. Thus, the nucleic acid sequences encoding the viral vector components are not presented in the same 5′ to 3′ orientation, such that the viral vector components cannot be produced from the same mRNA molecule. The reverse orientation may mean that at least two coding sequences for different vector components are presented in the ‘head-to-head’ and ‘tail-to-tail’ transcriptional orientations. This may be achieved by providing the coding sequence for one vector component, e.g. env, on one strand and the coding sequence for another vector component, e.g. rev, on the opposing strand of the modular construct. Preferably, when coding sequences for more than two vector components are present in the modular construct, at least two of the coding sequences are present in the reverse transcriptional orientation. Accordingly, when coding sequences for more than two vector components are present in the modular construct, each component may be orientated such that it is present in the opposite 5′ to 3′ orientation to all of the adjacent coding sequence(s) for other vector components to which it is adjacent, i.e. alternating 5′ to 3′ (or transcriptional) orientations for each coding sequence may be employed.

The modular construct for use according to the present invention may comprise nucleic acid sequences encoding two or more of the following vector components: gag-pol, rev, env, vector genome. The modular construct may comprise nucleic acid sequences encoding any combination of the vector components. In one embodiment, the modular construct may comprise nucleic acid sequences encoding:
i) the RNA genome of the retroviral vector and rev, or a functional substitute thereof;
ii) the RNA genome of the retroviral vector and gag-pol;
iii) the RNA genome of the retroviral vector and env;
iv) gag-pol and rev, or a functional substitute thereof;
v) gag-pol and env;
vi) env and rev, or a functional substitute thereof;
vii) the RNA genome of the retroviral vector, rev, or a functional substitute thereof, and gag-pol;
viii) the RNA genome of the retroviral vector, rev, or a functional substitute thereof, and env;
ix) the RNA genome of the retroviral vector, gag-pol and env; or
x) gag-pol, rev, or a functional substitute thereof, and env,
   wherein the nucleic acid sequences are in reverse and/or alternating orientations.

In one embodiment, a cell for producing retroviral vectors may comprise nucleic acid sequences encoding any one of the combinations i) to x) above, wherein the nucleic acid sequences are located at the same genetic locus and are in reverse and/or alternating orientations. The same genetic locus may refer to a single extrachromosomal locus in the cell, e.g. a single plasmid, or a single locus (i.e. a single insertion site) in the genome of the cell. The cell may be a stable or transient cell for producing retroviral vectors, e.g. lentiviral vectors.

The DNA expression construct can be a DNA plasmid (supercoiled, nicked or linearised), minicircle DNA (linear or supercoiled), plasmid DNA containing just the regions of interest by removal of the plasmid backbone by restriction enzyme digestion and purification, DNA generated using an enzymatic DNA amplification platform e.g. doggybone DNA (dbDNA™) where the final DNA used is in a closed ligated form or where it has been prepared (e.g restriction enzyme digestion) to have open cut ends.

In one embodiment, the lentiviral vector is derived from HIV-1, HIV-2, SIV, FIV, BIV, EIAV, CAEV or Visna lentivirus.

A “viral vector production cell”, “vector production cell”, or “production cell” is to be understood as a cell that is capable of producing a lentiviral vector or lentiviral vector particle. Lentiviral vector production cells may be “producer cells” or “packaging cells”. One or more DNA constructs of the viral vector system may be either stably integrated or episomally maintained within the viral vector production cell. Alternatively, all the DNA components of the viral vector system may be transiently transfected into the viral vector production cell. In yet another alternative, a production cell stably expressing some of the components may be transiently transfected with the remaining components required for vector production.

In one aspect of the invention as described herein, the U1 expression cassette is stably integrated into a cell according to the invention as described herein.

As used herein, the term “packaging cell” refers to a cell which contains the elements necessary for production of lentiviral vector particles but which lacks the vector genome. Optionally, such packaging cells contain one or more expression cassettes which are capable of expressing viral structural proteins (such as gag, gag/pol and env) and typically rev.

Producer cells/packaging cells can be of any suitable cell type. Producer cells are generally mammalian cells but can be, for example, insect cells.

As used herein, the term “producer cell” or “vector producing/producer cell” refers to a cell which contains all the elements necessary for production of lentiviral vector particles. The producer cell may be either a stable producer cell line or derived transiently or may be a stable packaging cell wherein the retroviral genome is transiently expressed.

The vector production cells may be cells cultured in vitro such as a tissue culture cell line. Suitable cell lines include, but are not limited to, mammalian cells such as murine fibroblast derived cell lines or human cell lines. Preferably the vector production cells are derived from a human cell line.

### Cells and Production Methods

Another aspect of the invention relates to a method for producing lentiviral vectors comprising introducing the nucleotide sequences described herein into a cell (e.g. a production cell) and culturing the cell under conditions suitable for the production of the lentiviral vectors.

Therefore, in one aspect the present invention provides a method for producing a lentiviral vector, comprising the steps of:
a) introducing nucleotide sequences encoding vector components and at least one nucleotide sequence encoding a modified U1 snRNA of the invention, into a cell;
b) optionally selecting for a cell which comprises said nucleotide sequences encoding vector components and at least one nucleotide sequence encoding a modified U1 snRNA of the invention;
c) further culturing the cell under conditions in which said vector components are co-expressed with said modified U1 snRNA and the lentiviral vector is produced; and
d) optionally isolating the lentiviral vector.

In a further aspect, the present invention provides a method for producing a lentiviral vector, comprising the steps of:
a) introducing nucleotide sequences encoding vector components and at least one nucleotide sequence encoding a modified U1 snRNA of the invention, into a cell;
b) selecting for a cell which comprises said nucleotide sequences encoding vector components and at least one nucleotide sequence encoding a modified U1 snRNA of the invention;
c) optionally introducing a nucleic acid vector which is different to the nucleotide sequence encoding a modified U1 snRNA of the invention into the selected cell;
d) further culturing the cell under conditions in which the lentiviral vector is produced; and
e) optionally isolating the lentiviral vector.

In the methods of the invention, the vector components may include gag, env, rev and/or the RNA genome of the lentiviral vector. The nucleotide sequences encoding vector components and at least one nucleotide sequence encoding a modified U1 snRNA of the invention may be introduced into the cell either simultaneously or sequentially in any order. The nucleotide sequences encoding vector components may be introduced into the cell prior to at least one nucleotide sequence encoding a modified U1 snRNA of the invention. The at least one nucleotide sequence encoding a modified U1 snRNA of the invention may be introduced into the cell prior to nucleotide sequences encoding vector components.

In some embodiments, the lentiviral vector may be replication-defective.

In a further aspect, the present invention provides a lentiviral vector produced by any method of the invention.

In a further aspect, the present invention provides the use of a modified U1 snRNA of the invention or nucleotide sequence encoding a modified U1 snRNA of the invention or a production cell of the invention for the production of a lentiviral vector.

The lentiviral vector production may involve co-expression of a modified U1 snRNA of the invention with vector components in a suitable production cell as described herein.

In a further aspect the present invention provides a method for generating a production cell for producing lentiviral vectors, comprising the steps of:
a) introducing nucleotide sequences encoding vector components including gag, env, rev and the RNA genome of the lentiviral vector, and at least one nucleotide sequence encoding a modified U1 snRNA of the invention, into a cell; and
b) optionally selecting for a cell which comprises said nucleotide sequences encoding vector components and at least one nucleotide sequence encoding a modified U1 snRNA of the invention.

In a further aspect the present invention provides a method for generating a stable production cell for producing lentiviral vectors, comprising the steps of:
a) introducing nucleotide sequences encoding vector components including gag, env, rev and the RNA genome of the lentiviral vector, and at least one nucleotide sequence encoding a modified U1 snRNA of the invention, into a cell; and
b) selecting for a cell which comprises said nucleotide sequences encoding vector components or at least one nucleotide sequence encoding at least one modified U1 snRNA of the invention.

In a further aspect, the invention provides a method for generating a transient production cell for producing lentiviral vectors, comprising introducing nucleotide sequences encoding vector components including gag, env, rev and the RNA genome of the lentiviral vector, and at least one nucleotide sequence encoding a modified U1 snRNA of the invention, into a cell.

In a further aspect, the invention provides a cell for producing lentiviral vectors produced by any method of the invention.

In a further aspect, the invention provides a stable production cell for producing lentiviral vectors produced by any method of the invention.

In a further aspect, the invention provides a transient production cell for producing lentiviral vectors produced by any method of the invention.

In a further aspect, the invention provides a cell for producing lentiviral vectors comprising at least one nucleotide sequence encoding a modified U1 snRNA of the invention.

In a further aspect, the present invention provides a stable production cell for producing lentiviral vectors comprising at least one nucleotide sequence encoding a modified U1 snRNA of the invention.

In a further aspect, the invention provides a transient production cell for producing lentiviral vectors comprising at least one nucleotide sequence encoding a modified U1 snRNA of the invention.

In some embodiments, the stable or transient production cell for producing lentiviral vectors comprises 1, 5, 10, 15, 20 or 30 stably integrated nucleotide sequences encoding modified U1 snRNAs of the invention.

In some embodiments of the methods and uses of the invention, binding of a modified U1 snRNA of the invention to the nucleotide sequence within the packaging region of a lentiviral vector genome sequence enhances lentiviral vector titre during lentiviral vector production relative to lentiviral vector production in the absence of a modified U1 snRNA of the invention. Thus, production of a lentiviral vector in the presence of a modified U1 snRNA of the invention enhances lentiviral vector titre relative to lentiviral vector production in the absence of a modified U1 snRNA of the invention. A suitable assay for the measurement of lentiviral vector titre is as described herein. Suitably, the lentiviral vector production involves co-expression of said modified U1 snRNA with vector components including gag, env, rev and the RNA genome of the lentiviral vector. In some embodiments, the enhancement of lentiviral vector titre occurs in the presence or absence of a functional 5′LTR polyA site. In some embodiments, the enhancement of lentiviral vector titres mediated by a modified U1 snRNA of the invention is independent of polyA site suppression in the 5′LTR of the vector genome.

In some embodiments of the methods and uses of the invention, the binding of a modified U1 snRNA of the invention to the nucleotide sequence within the packaging region of a lentiviral vector genome sequence may increase lentiviral vector titre during lentiviral vector production by at least 30% relative to lentiviral vector production in the absence of a modified U1 snRNA of the invention. Suitably, the binding of a modified U1 snRNA of the invention to the nucleotide sequence within the packaging region of a lentiviral vector genome sequence may increase lentiviral vector titre during lentiviral vector production by at least 35% (suitably at least 40%, 45%, 50%, 60%, 70%, 100%, 150%, 200%, 250%, 300%, 350%, 400%, 450%, 500%, 550%, 600%, 650%, 700%, 750%, 800%, 850%, 900%, 950% or 1000%) relative to lentiviral vector production in the absence of a modified U1 snRNA of the invention.

In some embodiments of the methods and uses of the invention, suitable production cells or cells for producing a lentiviral vector are those cells which are capable of producing viral vectors or viral vector particles when cultured under appropriate conditions. Thus, the cells typically comprise nucleotide sequences encoding vector components, which may include gag, env, rev and the RNA genome of the lentiviral vector. Suitable cell lines include, but are not limited to, mammalian cells such as murine fibroblast derived cell lines or human cell lines. They are generally mammalian, including human cells, for example HEK293T, HEK293, CAP, CAP-T or CHO cells, but can be, for example, insect cells such as SF9 cells. Preferably, the vector production cells are derived from a human cell line. Accordingly, such suitable production cells may be employed in any of the methods or uses of the present invention.

Methods for introducing nucleotide sequences into cells are well known in the art and have been described previously. Thus, the introduction into a cell of nucleotide sequences encoding vector components including gag, env, rev and the RNA genome of the lentiviral vector, and at least one nucleotide sequence encoding a modified U1 snRNA of the invention or at least one expression cassette of the invention, using conventional techniques in molecular and cell biology is within the capabilities of a person skilled in the art.

Stable production cells may be packaging or producer cells. To generate producer cells from packaging cells the vector genome DNA construct may be introduced stably or transiently. Packaging/producer cells can be generated by transducing a suitable cell line with a retroviral vector which expresses one of the components of the vector, i.e. a genome, the gag-pol components and an envelope as described in WO 2004/022761.

Alternatively, the nucleotide sequence can be transfected into cells and then integration into the production cell genome occurs infrequently and randomly. The transfection methods may be performed using methods well known in the art. For example, a stable transfection process may employ constructs which have been engineered to aid concatemerisation. In another example, the transfection process may be performed using calcium phosphate or commercially available formulations such as Lipofectamine™ 2000CD (Invitrogen, Calif.), FuGENE® HD or polyethylenimine (PEI). Alternatively nucleotide sequences may be introduced into the production cell via electroporation. The skilled person will be aware of methods to encourage integration of the nucleotide sequences into production cells. For example, linearising a nucleic acid construct can help if it is naturally circular. Less random integration methodologies may involve the nucleic acid construct comprising of areas of shared homology with the endogenous chromosomes of the mammalian host cell to guide integration to a selected site within the endogenous genome. Furthermore, if recombination sites are present on the construct then these can be used for targeted recombination. For example, the nucleic acid construct may contain a loxP site which allows for targeted integration when combined with Cre recombinase (i.e. using the Cre/lox system derived from P1 bacteriophage). Alternatively or additionally, the recombination site is an att site (e.g. from A phage), wherein the att site permits site-directed integration in the presence of a lambda integrase. This would allow the lentiviral genes to be targeted to a locus within the host cellular genome which allows for high and/or stable expression.

Other methods of targeted integration are well known in the art. For example, methods of inducing targeted cleavage of genomic DNA can be used to encourage targeted recombination at a selected chromosomal locus. These methods often involve the use of methods or systems to induce a double strand break (DSB) e.g. a nick in the endogenous genome to induce repair of the break by physiological mechanisms such as non-homologous end joining (NHEJ). Cleavage can occur through the use of specific nucleases such as engineered zinc finger nucleases (ZFN), transcription-activator like effector nucleases (TALENs), using CRISPR/Cas9 systems with an engineered crRNA/tracr RNA (′single guide RNA′) to guide specific cleavage, and/or using nucleases based on the Argonaute system (e.g., from T. thermophilus).

Packaging/producer cell lines can be generated by integration of nucleotide sequences using methods of just lentiviral transduction or just nucleic acid transfection, or a combination of both can be used.

Methods for generating retroviral vectors from production cells and in particular the processing of retroviral vectors are described in WO 2009/153563.

In one embodiment, the production cell may comprise the RNA-binding protein (e.g. tryptophan RNA-binding attenuation protein, TRAP) and/or the Tet Repressor (TetR) protein or alternative regulatory proteins (e.g. CymR).

Production of lentiviral vector from production cells can be via transfection methods, from production from stable cell lines which can include induction steps (e.g. doxycycline induction) or via a combination of both. The transfection methods may be performed using methods well known in the art, and examples have been described previously.

Production cells, either packaging or producer cell lines or those transiently transfected with the lentiviral vector encoding components are cultured to increase cell and virus numbers and/or virus titres. Culturing a cell is performed to enable it to metabolize, and/or grow and/or divide and/or produce viral vectors of interest according to the invention. This can be accomplished by methods well known to persons skilled in the art, and includes but is not limited to providing nutrients for the cell, for instance in the appropriate culture media. The methods may comprise growth adhering to surfaces, growth in suspension, or combinations thereof. Culturing can be done for instance in tissue culture flasks, tissue culture multiwell plates, dishes, roller bottles, wave bags or in bioreactors, using batch, fed-batch, continuous systems and the like. In order to achieve large scale production of viral vector through cell culture it is preferred in the art to have cells capable of growing in suspension. Suitable conditions for culturing cells are known (see e.g. Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9).

Preferably cells are initially ‘bulked up’ in tissue culture flasks or bioreactors and subsequently grown in multi-layered culture vessels or large bioreactors (greater than 50 L) to generate the vector producing cells of the present invention.

Preferably cells are grown in an adherent mode to generate the vector producing cells of the present invention.

Preferably cells are grown in a suspension mode to generate the vector producing cells of the present invention.

### Major Splice Donor

Mutation of the major splice donor site in the packaging region of the RNA genome of a viral vector has been shown to be detrimental to vector production titres, and additionally activate a cryptic splice donor (crSD) immediately adjacent to the MSD. Aberrant splicing from the MSD or CrSD leads to production of spliced RNA that cannot be packaged into vector virions. Splicing from the MSD to cellular transcripts from transcription read-through products derived from integrated vectors in transduced cells has also been reported, raising safety concerns. The present inventors describe novel mutations within the MSD splicing region that lead to less pronounced reduction in vector titres (in the absence of modified U1 snRNA) leading to further increases in titres in the presence of modified U1 snRNA. s. Such a mutation or deletion of the major splice donor site may have additional improved effects on vector titre to those described herein, and may be used in combination with any other aspect of the invention as described herein.

RNA splicing is catalysed by a large RNA-protein complex called the spliceosome, which is comprised of five small nuclear ribonucleoproteins (snRNPs). The borders between introns and exons are marked by specific nucleotide sequences within a pre-mRNA, which delineate where splicing will occur. Such boundaries are referred to as “splice sites.” The term “splice site” refers to polynucleotides that are capable of being recognized by the splicing machinery of a eukaryotic cell as suitable for being cut and/or ligated to another splice site.

Splice sites allow for the excision of introns present in a pre-mRNA transcript. Typically, the 5′ splice boundary is referred to as the “splice donor site” or the “5′ splice site,” and the 3′ splice boundary is referred to as the “splice acceptor site” or the “3′ splice site.” Splice sites include, for example, naturally occurring splice sites, engineered or synthetic splice sites, canonical or consensus splice sites, and/or non-canonical splice sites, for example, cryptic splice sites.

Splice acceptor sites generally consist of three separate sequence elements: the branch point or branch site, a polypyrimidine tract and the acceptor consensus sequence. The branch point consensus sequence in eukaryotes is YNYTRAC (where Y is a pyrimidine, N is any nucleotide, and R is a purine). The 3′ acceptor splice site consensus sequence is YAG (where Y is a pyrimidine) (see, e.g., Griffiths et al., eds., Modern Genetic Analysis, 2nd edition, W.H. Freeman and Company, New York (2002)). The 3′ splice acceptor site typically is located at the 3′ end of an intron.

As such, the major splice donor site may be inactivated in the nucleotide sequence encoding the RNA genome of the lentiviral vector for use in the present invention.

In one aspect the invention also provides a cell according to the invention as described herein, wherein the major splice donor site in the RNA genome of the lentiviral vector is inactivated, for example is mutated or deleted.

In one aspect is provided a nucleotide sequence encoding the RNA genome of a lentiviral vector, wherein the major splice donor site in the RNA genome of said lentiviral vector is inactivated, for example is mutated or deleted.

The terms “canonical splice site” or “consensus splice site” may be used interchangeably and refer to splice sites that are conserved across species.

Consensus sequences for the 5′ donor splice site and the 3′ acceptor splice site used in eukaryotic RNA splicing are well known in the art. These consensus sequences include nearly invariant dinucleotides at each end of the intron: GT at the 5′ end of the intron, and AG at the 3′ end of an intron.

The canonical splice donor site consensus sequence may be (for DNA) AG/GTRAGT (where A is adenosine, T is thymine, G is guanine, C is cytosine, R is a purine and “I” indicates the cleavage site). This conforms to the more general splice donor consensus sequence MAGGURR described herein. It is well known in the art that a splice donor sequence may deviate from this consensus, especially in viral genomes where other constraints bear on the same sequence, such as secondary structure for example within a vRNA packaging region. Non-canonical splice sites are also well known in the art, albeit they occur rarely compared to the canonical splice donor consensus sequence.

By “major splice donor site” is meant the first (dominant) splice donor site in the viral vector genome, encoded and embedded within the native viral RNA packaging sequence typically located in the 5′ region of the viral vector nucleotide sequence.

In one aspect the nucleotide sequence does not contain an active major splice donor site, that is splicing does not occur from the major splice donor site in said nucleotide sequence, and splicing activity from the major splice donor site is ablated.

The major splice donor site is located in the 5′ packaging region of a lentiviral genome.

In the case of the HIV-1 virus, the major splice donor consensus sequence is (for DNA) TG/GTRAGT (where A is adenosine, T is thymine, G is guanine, C is cytosine, R is a purine and “I” indicates the cleavage site).

In one aspect of the invention, the splice donor region, i.e. the region of the vector genome which comprises the major splice donor site prior to mutation may have the following sequence:

| (SEQ ID NO: 1) | |
|---|---|
| | GGGGCGGCGACTGGTGAGTACGCCAAAAAT |

In one aspect of the invention the mutated splice donor region may comprise the sequence:

| | (MSD-2KO) |
|---|---|
| SEQ ID NO: 2 | |
| | GGGGCGGCGACTGCAGACAACGCCAAAAAT |

In one aspect of the invention the mutated splice donor region may comprise the sequence:

| | (MSD-2KOv2) |
|---|---|
| SEQ ID NO: 11 | |
| | GGGGCGGCGAGTGGAGACTACGCCAAAAAT |

In one aspect of the invention the mutated splice donor region may comprise the sequence:

| | (MSD-2KOm5) |
|---|---|
| SEQ ID NO: 12 | |
| | GGGGAAGGCAACAGATAAATATGCCTTAAAAT |

In one aspect of the invention prior to modification the splice donor region may comprise the sequence:

| (SEQ ID NO: 9) | |
|---|---|
| | GGCGACTGGTGAGTACGCC |

This sequence is also referred to herein as the “stem loop 2” region (SL2). This sequence may form a stem loop structure in the splice donor region of the vector genome. In one aspect of the invention this sequence (SL2) may have been deleted from the nucleotide sequence according to the invention as described herein.

As such, the invention encompasses a nucleotide sequence that does not comprise SL2. The invention encompasses a nucleotide sequence that does not comprise a sequence according to SEQ ID NO:9.

In one aspect of the invention the major splice donor site may have the following consensus sequence, wherein R is a purine and “I” is the cleavage site:

(SEQ ID NO: 3)
TG/GTRAGT

In one aspect, R may be guanine (G).

In one aspect of the invention, the major splice donor and cryptic splice donor region may have the following core sequence, wherein “/” are the cleavage sites at the major splice donor and cryptic splice donor sites:

(SEQ ID NO: 13)
/GTGA/GTA

In one aspect of the invention the MSD-mutated vector genome may have at least two mutations in the major splice donor and cryptic splice donor ‘region’ (SEQ ID NO:13), wherein the first and second ‘GT’ nucleotides are the immediately 3′ of the major splice donor and cryptic splice donor nucleotides respectively

In one aspect of the invention the major splice donor consensus sequence is CTGGT (SEQ ID NO:4). The major splice donor site may contain the sequence CTGGT.

In one aspect the nucleotide sequence, prior to inactivation of the splice sites, comprises the sequence as set forth in any of SEQ ID NOs: 1, 3, 4, 9, 10 and/or 13.

In one aspect the nucleotide sequence comprises an inactivated major splice donor site which would otherwise have a cleavage site between nucleotides corresponding to nucleotides 13 and 14 of SEQ ID NO:1.

According to the invention as described herein, the nucleotide sequence also contains an inactive cryptic splice donor site. In one aspect the nucleotide sequence does not contain an active cryptic splice donor site adjacent to (3′ of) the major splice donor site, that is to say that splicing does not occur from the adjacent cryptic splice donor site, and splicing from the cryptic splice donor site is ablated.

The term “cryptic splice donor site” refers to a nucleic acid sequence which does not normally function as a splice donor site or is utilised less efficiently as a splice donor site due to the adjacent sequence context (e.g. the presence of a nearby ‘preferred’ splice donor), but can be activated to become a more efficient functioning splice donor site by mutation of the adjacent sequence (e.g. mutation of the nearby ‘preferred’ splice donor).

In one aspect the cryptic splice donor site is the first cryptic splice donor site 3′ of the major splice donor.

In one aspect the cryptic splice donor site is within 6 nucleotides of the major splice donor site on the 3′ side of the major splice donor site. Preferably the cryptic splice donor site is within 4 or 5, preferably 4, nucleotides of the major splice donor cleavage site.

In one aspect of the invention the cryptic splice donor site has the consensus sequence

(SEQ ID NO: 10)
TGAGT

In one aspect the nucleotide sequence comprises an inactivated cryptic splice donor site which would otherwise have a cleavage site between nucleotides corresponding to nucleotides 17 and 18 of SEQ ID NO:1.

In one aspect of the invention the major splice donor site and/or adjacent cryptic splice donor site contain a “GT” motif. In one aspect of the invention both the major splice donor site and adjacent cryptic splice donor site contain a “GT” motif which is mutated. The mutated GT motifs may inactivate splice activity from both the major splice donor site and adjacent cryptic splice donor site. An example of such a mutation is referred to herein as “MSD-2KO”.

In one aspect the splice donor region may comprise the following sequence:

(SEQ ID NO: 5)
CAGACA

For example, in one aspect the mutated splice donor region may comprise the following sequence:

(SEQ ID NO: 6)
GGCGACTGCAGACAACGCC

A further example of an inactivating mutation is referred to herein as “MSD-2KOv2”.

In one aspect the mutated splice donor region may comprise the following sequence:

(SEQ ID NO: 7)
GTGGAGACT

For example, in one aspect the mutated splice donor region may comprise the following sequence:

(SEQ ID NO: 8)
GGCGAGTGGAGACTACGCC

For example, in one aspect the mutated splice donor region may comprise the following sequence:

(SEQ ID NO: 14)
AAGGCAACAGATAAATATGCCTT

In one aspect the stem loop 2 region as described above may be deleted from the splice donor region, resulting in inactivation of both the major splice donor site and the adjacent cryptic splice donor site. Such a deletion is referred to herein as “ΔSL2”.

A variety of different types of mutations can be introduced into the nucleic acid sequence in order to inactivate the major and adjacent cryptic splice donor sites.

In one aspect the mutation is a functional mutation to ablate or suppress splicing activity in the splice region. The nucleotide sequence as described herein may contain a mutation or deletion in any of the nucleotides in any of SEQ ID NOs: 1, 3, 4, 9, 10 and/or 13. Suitable mutations will be known to one skilled in the art, and are described herein.

For example, a point mutation can be introduced into the nucleic acid sequence. The term “point mutation,” as used herein, refers to any change to a single nucleotide. Point mutations include, for example, deletions, transitions, and transversions; these can be classified as nonsense mutations, missense mutations, or silent mutations when present within protein coding sequence. A “nonsense” mutation produces a stop codon. A “missense” mutation produces a codon that encodes a different amino acid. A “silent” mutation produces a codon that encodes either the same amino acid or a different amino acid that does not alter the function of the protein. One or more point mutations can be introduced into the nucleic acid sequence comprising the cryptic splice donor site. For example, the nucleic acid sequence comprising the cryptic splice site can be mutated by introducing two or more point mutations therein.

At least two point mutations can be introduced in several locations within the nucleic acid sequence comprising the major splice donor and cryptic splice donor sites to achieve attenuation of splicing from the splice donor region. In one aspect the mutations may be within the four nucleotides at the splice donor cleavage site; in the canonical splice donor consensus sequence this is A¹G²/G³T⁴, wherein “/” is the cleavage site. It is well known in the art that a splice donor cleavage site may deviate from this consensus, especially in viral genomes where other constraints bear on the same sequence, such as secondary structure for example within a vRNA packaging region. It is well known that the G³T⁴ dinucleotide is generally the least variable sequence within the canonical splice donor consensus sequence, and mutations to the G³ and or T⁴ will most likely achieve the greatest attenuating effect. For example, for the major splice donor site in HIV-1 viral vector genomes this can be T¹G²/G³T⁴, wherein “I” is the cleavage site. For example, for the cryptic splice donor site in HIV-1 viral vector genomes this can be G¹A²/G³T⁴, wherein “/” is the cleavage site. Additionally, the point mutation(s) can be introduced adjacent to a splice donor site. For example, the point mutation can be introduced upstream or downstream of a splice donor site. In embodiments where the nucleic acid sequence comprising a major and/or cryptic splice donor site is mutated by introducing multiple point mutations therein, the point mutations can be introduced upstream and/or downstream of the cryptic splice donor site.

### Construction of Splice Site Mutants

Splice site mutants of the present invention may be constructed using a variety of techniques. For example, mutations may be introduced at particular loci by synthesising oligonucleotides containing a mutant sequence, flanked by restriction sites enabling ligation to fragments of the native sequence. Following ligation, the resulting reconstructed sequence comprises a derivative having the desired nucleotide insertion, substitution, or deletion.

Other known techniques allowing alterations of DNA sequence include recombination approaches such as Gibson assembly, Golden-gate cloning and In-fusion.

Alternatively, oligonucleotide-directed site-specific (or segment specific) mutagenesis procedures may be employed to provide an altered sequence according to the substitution, deletion, or insertion required. Deletion or truncation derivatives of splice site mutants may also be constructed by utilising convenient restriction endonuclease sites adjacent to the desired deletion.

Subsequent to restriction, overhangs may be filled in, and the DNA religated.

Exemplary methods of making the alterations set forth above are disclosed by Sambrook et al. (Molecular cloning: A Laboratory Manual, 2d Ed., Cold Spring Harbor Laboratory Press, 1989).

Splice site mutants may also be constructed utilising techniques of PCR mutagenesis, chemical mutagenesis, chemical mutagenesis (Drinkwater and Klinedinst, 1986) by forced nucleotide misincorporation (e.g., Liao and Wise, 1990), or by use of randomly mutagenised oligonucleotides (Horwitz et al., 1989).

The present invention also provides a method for producing a lentiviral vector nucleotide sequence, comprising the steps of:
(i) providing a nucleotide sequence encoding the RNA genome of a lentiviral vector as described herein; and
(ii) mutating the major splice donor site and cryptic splice donor site as described herein in said nucleotide sequence.

### Lentiviral Vectors

Lentiviruses are part of a larger group of retroviruses. A detailed list of lentiviruses may be found in Coffin et al (1997) “Retroviruses” Cold Spring Harbour Laboratory Press Eds: J M Coffin, S M Hughes, H E Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), feline immunodeficiency virus (FIV), Maedi visna virus (MVV) and bovine immunodeficiency virus (BIV). In one embodiment, the lentiviral vector is derived from HIV-1, HIV-2, SIV, FIV, BIV, EIAV, CAEV or Visna lentivirus.

In one embodiment the lentiviral vector is derived from HIV-1.

In one embodiment the lentiviral vector is derived from HIV-2.

In one embodiment the lentiviral vector is derived from EIAV.

In one embodiment the lentiviral vector is derived from SIV.

In one embodiment the lentiviral vector is derived from FIV.

In one embodiment the lentiviral vector is derived from BIV.

In one embodiment the lentiviral vector is derived from CAEV.

In one embodiment the lentiviral vector is derived from Visna lentivirus.

The lentivirus family differs from retroviruses in that lentiviruses have the capability to infect both dividing and non-dividing cells (Lewis et al (1992) EMBO J 11(8):3053-3058 and Lewis and Emerman (1994) J Virol 68 (1):510-516). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects or transduces target cells and expresses NOI.

The lentiviral vector may be used to replicate the NOI in a compatible target cell in vitro. Thus, described herein is a method of making proteins in vitro by introducing a vector of the invention into a compatible target cell in vitro and growing the target cell under conditions which result in expression of the NOI. Protein and NOI may be recovered from the target cell by methods well known in the art. Suitable target cells include mammalian cell lines and other eukaryotic cell lines.

In some aspects the vectors may have “insulators”—genetic sequences that block the interaction between promoters and enhancers, and act as a barrier reducing read-through from an adjacent gene.

In one embodiment the insulator is present between one or more of the lentiviral nucleic acid sequences to prevent promoter interference and read-thorough from adjacent genes. If the insulators are present in the vector between one or more of the lentiviral nucleic acid sequences, then each of these insulated genes may be arranged as individual expression units.

The basic structure of retroviral and lentiviral genomes share many common features such as a 5′ LTR and a 3′ LTR, between or within which are located a packaging signal to enable the genome to be packaged, a primer binding site, integration sites to enable integration into a target cell genome and gag/pol and env genes encoding the packaging components—these are polypeptides required for the assembly of viral particles. Lentiviruses have additional features, such as the rev gene and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a typical lentiviral vector as described herein, at least part of one or more protein coding regions essential for replication may be removed from the virus; for example, gag/pol and env may be absent or not functional. This makes the viral vector replication-defective.

The lentiviral vector may be derived from either a primate lentivirus (e.g. HIV-1) or a non-primate lentivirus (e.g. EIAV).

In general terms, a typical retroviral vector production system involves the separation of the viral genome from the essential viral packaging functions. These components are normally provided to the production cells on separate DNA expression cassettes (alternatively known as plasmids, expression plasmids, DNA constructs or expression constructs).

The vector genome comprises the NOI. Vector genomes typically require a packaging signal (ψ), the internal expression cassette harbouring the NOI, (optionally) a post-transcriptional element (PRE), typically a central polypurine tract (cppt), the 3′-ppu and a self-inactivating (SIN) LTR. The R-U5 regions are required for correct polyadenylation of both the vector genome RNA and NOI mRNA, as well as the process of reverse transcription. The vector genome may optionally include an open reading frame, as described in WO 2003/064665, which allows for vector production in the absence of rev.

The packaging functions include the gag/pol and env genes. These are required for the production of vector particles by the production cell. Providing these functions in trans to the genome facilitates the production of replication-defective viral vectors.

Production systems for gamma-retroviral vectors are typically 3-component systems requiring genome, gag/pol and env expression constructs. Production systems for HIV-1-based lentiviral vectors may additionally require the accessory gene rev to be provided and for the vector genome to include the rev-responsive element (RRE). EIAV-based lentiviral vectors do not require rev to be provided in trans if an open-reading frame (ORF) is present within the genome (see WO 2003/064665).

Usually both the “external” promoter (which drives the vector genome cassette) and “internal” promoter (which drives the NOI cassette) encoded within the vector genome cassette are strong eukaryotic or virus promoters, as are those driving the other vector system components. Examples of such promoters include CMV, EF1α, PGK, CAG, TK, SV40 and Ubiquitin promoters. Strong ‘synthetic’ promoters, such as those generated by DNA libraries (e.g. JeT promoter) may also be used to drive transcription. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VM D2), Tyrosine hydroxylase, neuronal-specific neuronal-specific enolase (NSE) promoter, astrocyte-specific glial fibrillary acidic protein (GFAP) promoter, human α1-antitrypsin (hAAT) promoter, phosphoenolpyruvate carboxykinase (PEPCK), liver fatty acid binding protein promoter, Flt-1 promoter, INF-β promoter, Mb promoter, SP-B promoter, SYN1 promoter, WASP promoter, SV40/hAIb promoter, SV40/CD43, SV40/CD45, NSE/RU5′ promoter, ICAM-2 promoter, GPIIb promoter, GFAP promoter, Fibronectin promoter, Endoglin promoter, Elastase-1 promoter, Desmin promoter, CD68 promoter, CD14 promoter and B29 promoter may be used to drive transcription.

Production of retroviral vectors involves either the transient co-transfection of the production cells with these DNA components or use of stable production cell lines wherein all the components are stably integrated within the production cell genome (e.g. Stewart H J, Fong-Wong L, Strickland I, Chipchase D, Kelleher M, Stevenson L, Thoree V, McCarthy J, Ralph G S, Mitrophanous K A and Radcliffe P A. (2011). Hum Gene Ther. March; 22 (3):357-69). An alternative approach is to use a stable packaging cell (into which the packaging components are stably integrated) and then transiently transfect in the vector genome plasmid as required (e.g. Stewart, H. J., M. A. Leroux-Carlucci, C. J. Sion, K. A. Mitrophanous and P. A. Radcliffe (2009). Gene Ther. June; 16 (6):805-14). It is also feasible that alternative, not complete, packaging cell lines could be generated (just one or two packaging components are stably integrated into the cell lines) and to generate vector the missing components are transiently transfected. The production cell may also express regulatory proteins such as a member of the tet repressor (TetR) protein group of transcription regulators (e.g.T-Rex, Tet-On, and Tet-Off), a member of the cumate inducible switch system group of transcription regulators (e.g. cumate repressor (CymR) protein), or an RNA-binding protein (e.g. TRAP—tryptophan-activated RNA-binding protein).

In one embodiment of the present invention, the viral vector is derived from EIAV. EIAV has the simplest genomic structure of the lentiviruses and is particularly preferred for use in the present invention. In addition to the gag/pol and env genes, EIAV encodes three other genes: tat, rev, and S2. Tat acts as a transcriptional activator of the viral LTR (Derse and Newbold (1993) Virology 194(2):530-536 and Maury et al (1994) Virology 200(2):632-642) and rev regulates and coordinates the expression of viral genes through rev-response elements (RRE) (Martarano et al. (1994) J Virol 68(5):3102-3111). The mechanisms of action of these two proteins are thought to be broadly similar to the analogous mechanisms in the primate viruses (Martarano et al. (1994) J Virol 68(5):3102-3111). The function of S2 is unknown. In addition, an EIAV protein, Ttm, has been identified that is encoded by the first exon of tat spliced to the env coding sequence at the start of the transmembrane protein. In an alternative embodiment of the present invention the viral vector is derived from HIV: HIV differs from EIAV in that it does not encode S2 but unlike EIAV it encodes vif, vpr, vpu and nef.

The term “recombinant retroviral or lentiviral vector” (RRV) refers to a vector with sufficient retroviral genetic information to allow packaging of an RNA genome, in the presence of packaging components, into a viral particle capable of transducing a target cell. Transduction of the target cell may include reverse transcription and integration into the target cell genome. The RRV carries non-viral coding sequences which are to be delivered by the vector to the target cell. A RRV is incapable of independent replication to produce infectious retroviral particles within the target cell. Usually the RRV lacks a functional gag/pol and/or env gene, and/or other genes essential for replication.

Preferably the RRV vector of the present invention has a minimal viral genome.

As used herein, the term “minimal viral genome” means that the viral vector has been manipulated so as to remove the non-essential elements whilst retaining the elements essential to provide the required functionality to infect, transduce and deliver a NOI to a target cell. Further details of this strategy can be found in WO 1998/17815 and WO 99/32646. A minimal EIAV vector lacks tat, S2 genes, and optionally rev, and neither are these genes provided in trans in the production system. A minimal HIV vector lacks vif, vpr, vpu, tat and nef.

The expression plasmid used to produce the vector genome within a production cell may include transcriptional regulatory control sequences operably linked to the retroviral genome to direct transcription of the genome in a production cell/packaging cell. All 3^{rd} generation lentiviral vectors are deleted in the 5′ U3 enhancer-promoter region, and transcription of the vector genome RNA is driven by heterologous promoter such as another viral promoter, for example the CMV promoter, as discussed below. This feature enables vector production independently of tat. Some lentiviral vector genomes require additional sequences for efficient virus production. For example, particularly in the case of HIV, RRE sequences may be included. However the requirement for RRE on the (separate) GagPol cassette (and dependence on rev which is provided in trans) may be reduced or eliminated by codon optimisation of the GagPol ORF. Further details of this strategy can be found in WO 2001/79518.

Alternative sequences which perform the same function as the rev/RRE system are also known. For example, a functional analogue of the rev/RRE system is found in the Mason Pfizer monkey virus. This is known as the constitutive transport element (CTE) and comprises an RRE-type sequence in the genome which is believed to interact with a factor in the infected cell. The cellular factor can be thought of as a rev analogue. Thus, CTE may be used as an alternative to the rev/RRE system. Any other functional equivalents of the Rev protein which are known or become available may be relevant to the invention. For example, it is also known that the Rex protein of HTLV-I can functionally replace the Rev protein of HIV-1. Rev and RRE may be absent or non-functional in the vector for use in the methods of the present invention; in the alternative rev and RRE, or functionally equivalent system, may be present.

As used herein, the term “functional substitute” means a protein or sequence having an alternative sequence which performs the same function as another protein or sequence. The term “functional substitute” is used interchangeably with “functional equivalent” and “functional analogue” herein with the same meaning.

### SIN Vectors

The lentiviral vectors as described herein may be used in a self-inactivating (SIN) configuration in which the viral enhancer and promoter sequences have been deleted. SIN vectors can be generated and transduce non-dividing target cells in vivo, ex vivo or in vitro with an efficacy similar to that of non-SIN vectors. The transcriptional inactivation of the long terminal repeat (LTR) in the SIN provirus should prevent mobilization of vRNA, and is a feature that further diminishes the likelihood of formation of replication-competent virus. This should also enable the regulated expression of genes from internal promoters by eliminating any cis-acting effects of the LTR.

By way of example, self-inactivating retroviral vector systems have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive ‘provirus’. However, any promoter(s) internal to the LTRs in such vectors will still be transcriptionally active. This strategy has been employed to eliminate effects of the enhancers and promoters in the viral LTRs on transcription from internally placed genes. Such effects include increased transcription or suppression of transcription. This strategy can also be used to eliminate downstream transcription from the 3′ LTR into genomic DNA. This is of particular concern in human gene therapy where it is important to prevent the adventitious activation of any endogenous oncogene. Yu et al., (1986) PNAS 83: 3194-98; Marty et al., (1990) Biochimie 72: 885-7; Naviaux et al., (1996) J. Virol. 70: 5701-5; Iwakuma et al., (1999) Virol. 261: 120-32; Deglon et al., (2000) Human Gene Therapy 11: 179-90. SIN lentiviral vectors are described in U.S. Pat. Nos. 6,924,123 and 7,056,699.

### Replication-Defective Lentiviral Vectors

In the genome of a replication-defective lentiviral vector the sequences of gag/pol and/or env may be mutated and/or not functional.

In a typical lentiviral vector as described herein, at least part of one or more coding regions for proteins essential for virus replication may be removed from the vector. This makes the viral vector replication-defective. Portions of the viral genome may also be replaced by a NOI in order to generate a vector comprising an NOI which is capable of transducing a non-dividing target cell and/or integrating its genome into the target cell genome.

In one embodiment the lentiviral vectors are non-integrating vectors as described in WO 2006/010834 and WO 2007/071994.

In a further embodiment the vectors have the ability to deliver a sequence which is devoid of or lacking viral RNA. In a further embodiment a heterologous binding domain (heterologous to gag) located on the RNA to be delivered and a cognate binding domain on Gag or GagPol can be used to ensure packaging of the RNA to be delivered. Both of these vectors are described in WO 2007/072056.

### NOI and Polynucleotides

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing PCR under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

### Common Retroviral Vector Elements

### Promoters and Enhancers

Expression of a NOI and polynucleotide may be controlled using control sequences for example transcription regulation elements or translation repression elements, which include promoters, enhancers and other expression regulation signals (e.g. tet repressor (TetR) system) or the Transgene Repression In vector Production cell system (TRIP) or other regulators of NOIs described herein.

Prokaryotic promoters and promoters functional in eukaryotic cells may be used. Tissue-specific or stimuli-specific promoters may be used. Chimeric promoters may also be used comprising sequence elements from two or more different promoters.

Suitable promoting sequences are strong promoters including those derived from the genomes of viruses, such as polyoma virus, adenovirus, fowlpox virus, bovine papilloma virus, avian sarcoma virus, cytomegalovirus (CMV), retrovirus and Simian Virus 40 (SV40), or from heterologous mammalian promoters, such as the actin promoter, EF1a, CAG, TK, SV40, ubiquitin, PGK or ribosomal protein promoter. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VMD2), Tyrosine hydroxylase, neuronal-specific neuronal-specific enolase (NSE) promoter, astrocyte-specific glial fibrillary acidic protein (GFAP) promoter, human α1-antitrypsin (hAAT) promoter, phosphoenolpyruvate carboxykinase (PEPCK), liver fatty acid binding protein promoter, Flt-1 promoter, INF-β promoter, Mb promoter, SP-B promoter, SYN1 promoter, WASP promoter, SV40/hAIb promoter, SV40/CD43, SV40/CD45, NSE/RU5′ promoter, ICAM-2 promoter, GPIIb promoter, GFAP promoter, Fibronectin promoter, Endoglin promoter, Elastase-1 promoter, Desmin promoter, CD68 promoter, CD14 promoter and B29 promoter may be used to drive transcription.

Transcription of a NOI may be increased further by inserting an enhancer sequence into the vector. Enhancers are relatively orientation- and position-independent; however, one may employ an enhancer from a eukaryotic cell virus, such as the SV40 enhancer and the CMV early promoter enhancer. The enhancer may be spliced into the vector at a position 5′ or 3′ to the promoter, but is preferably located at a site 5′ from the promoter.

The promoter can additionally include features to ensure or to increase expression in a suitable target cell. For example, the features can be conserved regions e.g. a Pribnow Box or a TATA box. The promoter may contain other sequences to affect (such as to maintain, enhance or decrease) the levels of expression of a nucleotide sequence. Suitable other sequences include the Sh1-intron or an ADH intron. Other sequences include inducible elements, such as temperature, chemical, light or stress inducible elements. Also, suitable elements to enhance transcription or translation may be present.

### Regulators of NOIs

A complicating factor in the generation of retroviral packaging/producer cell lines and retroviral vector production is that constitutive expression of certain retroviral vector components and NOIs are cytotoxic leading to death of cells expressing these components and therefore inability to produce vector. Therefore, the expression of these components (e.g. gag-pol and envelope proteins such as VSV-G) can be regulated. The expression of other non-cytotoxic vector components, e.g. rev, can also be regulated to minimise the metabolic burden on the cell. Thus the modular constructs or nucleotide sequences encoding vector components and/or cells as described herein may comprise cytotoxic and/or non-cytotoxic vector components associated with at least one regulatory element. As used herein, the term “regulatory element” refers to any element capable of affecting, either increasing or decreasing, the expression of an associated gene or protein. A regulatory element includes a gene switch system, transcription regulation element and translation repression element

A number of prokaryotic regulator systems have been adapted to generate gene switches in mammalian cells. Many retroviral packaging and producer cell lines have been controlled using gene switch systems (e.g. tetracycline and cumate inducible switch systems) thus enabling expression of one or more of the retroviral vector components to be switched on at the time of vector production. Gene switch systems include those of the (TetR) protein group of transcription regulators (e.g. T-Rex, Tet-On, and Tet-Off), those of the cumate inducible switch system group of transcription regulators (e.g. CymR protein) and those involving an RNA-binding protein (e.g. TRAP).

One such tetracycline-inducible system is the tetracycline repressor (TetR) system based on the T-REx™ system. By way of example, in such a system tetracycline operators (TetO₂) are placed in a position such that the first nucleotide is 10 bp from the 3′ end of the last nucleotide of the TATATAA element of the human cytomegalovirus major immediate early promoter (hCMVp) then TetR alone is capable of acting as a repressor (Yao F, Svensjo T, Winkler T, Lu M, Eriksson C, Eriksson E., 1998, Hum Gene Ther 9: 1939-1950). In such a system the expression of the NOI can be controlled by a CMV promoter into which two copies of the TetO₂ sequence have been inserted in tandem. TetR homodimers, in the absence of an inducing agent (tetracycline or its analogue doxycycline [dox]), bind to the TetO₂ sequences and physically block transcription from the upstream CMV promoter. When present, the inducing agent binds to the TetR homodimers, causing allosteric changes such that it can no longer bind to the TetO₂ sequences, resulting in gene expression. The TetR gene may be codon optimised as this was found to improve translation efficiency resulting in tighter control of TetO₂ controlled gene expression.

The TRIP system is described in WO 2015/092440 and provides another way of repressing expression of the NOI in the production cells during vector production. The TRAP-binding sequence (e.g. TRAP-tbs) interaction forms the basis for a transgene protein repression system for the production of retroviral vectors, when a constitutive and/or strong promoter, including a tissue-specific promoter, driving the transgene is desirable and particularly when expression of the transgene protein in production cells leads to reduction in vector titres and/or elicits an immune response in vivo due to viral vector delivery of transgene-derived protein (Maunder et al, Nat Commun. (2017) Mar. 27; 8).

Briefly, the TRAP-tbs interaction forms a translational block, repressing translation of the transgene protein (Maunder et al, Nat Commun. (2017) Mar. 27; 8). The translational block is only effective in production cells and as such does not impede the DNA- or RNA-based vector systems. The TRiP system is able to repress translation when the transgene protein is expressed from a constitutive and/or strong promoter, including a tissue-specific promoter from single- or multi cistronic mRNA. It has been demonstrated that unregulated expression of transgene protein can reduce vector titres and affect vector product quality. Repression of transgene protein for both transient and stable PaCL/PCL vector production systems is beneficial for production cells to prevent a reduction in vector titres: where toxicity or molecular burden issues may lead to cellular stress; where transgene protein elicits an immune response in vivo due to viral vector delivery of transgene-derived protein; where the use of gene-editing transgenes may result in on/off target affects; where the transgene protein may affect vector and/or envelope glycoprotein exclusion.

### Envelope and Pseudotyping

In one preferred aspect, the lentiviral vector as described herein has been pseudotyped. In this regard, pseudotyping can confer one or more advantages. For example, the env gene product of the HIV based vectors would restrict these vectors to infecting only cells that express a protein called CD4. But if the env gene in these vectors has been substituted with env sequences from other enveloped viruses, then they may have a broader infectious spectrum (Verma and Somia (1997) Nature 389(6648):239-242). By way of example, workers have pseudotyped an HIV based vector with the glycoprotein from VSV (Verma and Somia (1997) Nature 389(6648):239-242).

In another alternative, the Env protein may be a modified Env protein such as a mutant or engineered Env protein. Modifications may be made or selected to introduce targeting ability or to reduce toxicity or for another purpose (Valsesia-Wittman et al 1996 J Virol 70: 2056-64; Nilson et al (1996) Gene Ther 3(4):280-286; and Fielding et al (1998) Blood 91(5):1802-1809 and references cited therein).

The vector may be pseudotyped with any molecule of choice.

As used herein, “env” shall mean an endogenous lentiviral envelope or a heterologous envelope, as described herein.

### VSV-G

The envelope glycoprotein (G) of Vesicular stomatitis virus (VSV), a rhabdovirus, is an envelope protein that has been shown to be capable of pseudotyping certain enveloped viruses and viral vector virions.

Its ability to pseudotype MoMLV-based retroviral vectors in the absence of any retroviral envelope proteins was first shown by Emi et al. (1991) Journal of Virology 65:1202-1207. WO 1994/294440 teaches that retroviral vectors may be successfully pseudotyped with VSV-G. These pseudotyped VSV-G vectors may be used to transduce a wide range of mammalian cells. More recently, Abe et al. (1998) J Virol 72(8) 6356-6361 teach that non-infectious retroviral particles can be made infectious by the addition of VSV-G.

Burns et al. (1993) Proc. Natl. Acad. Sci. USA 90:8033-7 successfully pseudotyped the retrovirus MLV with VSV-G and this resulted in a vector having an altered host range compared to MLV in its native form. VSV-G pseudotyped vectors have been shown to infect not only mammalian cells, but also cell lines derived from fish, reptiles and insects (Burns et al. (1993) ibid). They have also been shown to be more efficient than traditional amphotropic envelopes for a variety of cell lines (Yee et al., (1994) Proc. Natl. Acad. Sci. USA 91:9564-9568, Emi et al. (1991) Journal of Virology 65:1202-1207). VSV-G protein can be used to pseudotype certain retroviruses because its cytoplasmic tail is capable of interacting with the retroviral cores.

The provision of a non-retroviral pseudotyping envelope such as VSV-G protein gives the advantage that vector particles can be concentrated to a high titre without loss of infectivity (Akkina et al. (1996) J. Virol. 70:2581-5). Retrovirus envelope proteins are apparently unable to withstand the shearing forces during ultracentrifugation, probably because they consist of two non-covalently linked subunits. The interaction between the subunits may be disrupted by the centrifugation. In comparison the VSV glycoprotein is composed of a single unit. VSV-G protein pseudotyping can therefore offer potential advantages for both efficient target cell infection/transduction and during manufacturing processes.

WO 2000/52188 describes the generation of pseudotyped retroviral vectors, from stable producer cell lines, having vesicular stomatitis virus-G protein (VSV-G) as the membrane-associated viral envelope protein, and provides a gene sequence for the VSV-G protein.

### Ross River Virus

The Ross River viral envelope has been used to pseudotype a non-primate lentiviral vector (FIV) and following systemic administration predominantly transduced the liver (Kang et al., 2002, J. Virol., 76:9378-9388). Efficiency was reported to be 20-fold greater than obtained with VSV-G pseudotyped vector, and caused less cytotoxicity as measured by serum levels of liver enzymes suggestive of hepatotoxicity.

### Baculovirus GP64

The baculovirus GP64 protein has been shown to be an alternative to VSV-G for viral vectors used in the large-scale production of high-titre virus required for clinical and commercial applications (Kumar M, Bradow B P, Zimmerberg J (2003) Hum Gene Ther. 14(1):67-77). Compared with VSV-G-pseudotyped vectors, GP64-pseudotyped vectors have a similar broad tropism and similar native titres. Because, GP64 expression does not kill cells, HEK293T-based cell lines constitutively expressing GP64 can be generated.

### Alternative Envelopes

Other envelopes which give reasonable titre when used to pseudotype EIAV include Mokola, Rabies, Ebola and LCMV (lymphocytic choriomeningitis virus). Intravenous infusion into mice of lentivirus pseudotyped with 4070A led to maximal gene expression in the liver.

### Packaging Sequence

As utilized within the context of the present invention the term “packaging signal”, which is referred to interchangeably as “packaging sequence” or “psi”, is used in reference to the non-coding, cis-acting sequence required for encapsidation of retroviral RNA strands during viral particle formation. In HIV-1, this sequence has been mapped to loci extending from upstream of the major splice donor site (SD) to at least the gag start codon (some or all of the 5′ sequence of gag to nucleotide 688 may be included). In EIAV the packaging signal comprises the R region into the 5′ coding region of Gag.

As used herein, the term “extended packaging signal” or “extended packaging sequence” refers to the use of sequences around the psi sequence with further extension into the gag gene. The inclusion of these additional packaging sequences may increase the efficiency of insertion of vector RNA into viral particles.

Feline immunodeficiency virus (FIV) RNA encapsidation determinants have been shown to be discrete and non-continuous, comprising one region at the 5′ end of the genomic mRNA (R-U5) and another region that mapped within the proximal 311 nt of gag (Kaye et al., J Virol. October; 69(10):6588-92 (1995).

### Internal Ribosome Entry Site (IRES)

Insertion of IRES elements allows expression of multiple coding regions from a single promoter (Adam et al (as above); Koo et al (1992) Virology 186:669-675; Chen et al 1993 J. Virol 67:2142-2148). IRES elements were first found in the non-translated 5′ ends of picornaviruses where they promote cap-independent translation of viral proteins (Jang et al (1990) Enzyme 44: 292-309). When located between open reading frames in an RNA, IRES elements allow efficient translation of the downstream open reading frame by promoting entry of the ribosome at the IRES element followed by downstream initiation of translation.

A review on IRES is presented by Mountford and Smith (TIG May 1995 vol 11, No 5:179-184). A number of different IRES sequences are known including those from encephalomyocarditis virus (EMCV) (Ghattas, I. R., et al., Mol. Cell. Biol., 11:5848-5859 (1991); BiP protein [Macejak and Sarnow, Nature 353:91 (1991)]; the Antennapedia gene of Drosophila (exons d and e) [Oh, et al., Genes & Development, 6:1643-1653 (1992)] as well as those in polio virus (PV) [Pelletier and Sonenberg, Nature 334: 320-325 (1988); see also Mountford and Smith, TIG 11, 179-184 (1985)].

IRES elements from PV, EMCV and swine vesicular disease virus have previously been used in retroviral vectors (Coffin et al, as above).

The term “IRES” includes any sequence or combination of sequences which work as or improve the function of an IRES. The IRES(s) may be of viral origin (such as EMCV IRES, PV IRES, or FMDV 2A-like sequences) or cellular origin (such as FGF2 IRES, NRF IRES, Notch 2 IRES or EIF4 IRES).

In order for the IRES to be capable of initiating translation of each polynucleotide it should be located between or prior to the polynucleotides in the modular construct.

The nucleotide sequences utilised for development of stable cell lines require the addition of selectable markers for selection of cells where stable integration has occurred. These selectable markers can be expressed as a single transcription unit within the nucleotide sequence or it may be preferable to use IRES elements to initiate translation of the selectable marker in a polycistronic message (Adam et al 1991 J. Virol. 65, 4985).

### Genetic Orientation and Insulators

It is well known that nucleic acids are directional and this ultimately affects mechanisms such as transcription and replication in the cell. Thus genes can have relative orientations with respect to one another when part of the same nucleic acid construct.

In certain embodiments of the present invention, at least two nucleic acid sequences present at the same locus in the cell or construct can be in a reverse and/or alternating orientations. In other words, in certain embodiments of the invention at this particular locus, the pair of sequential genes will not have the same orientation. This can help prevent both transcriptional and translational read-through when the region is expressed within the same physical location of the host cell.

Having the alternating orientations benefits retroviral vector production when the nucleic acids required for vector production are based at the same genetic locus within the cell. This in turn can also improve the safety of the resulting constructs in preventing the generation of replication-competent retroviral vectors.

When nucleic acid sequences are in reverse and/or alternating orientations the use of insulators can prevent inappropriate expression or silencing of a NOI from its genetic surroundings.

The term “insulator” refers to a class of DNA sequence elements that when bound to insulator-binding proteins possess an ability to protect genes from surrounding regulator signals. There are two types of insulators: an enhancer blocking function and a chromatin barrier function. When an insulator is situated between a promoter and an enhancer, the enhancer-blocking function of the insulator shields the promoter from the transcription-enhancing influence of the enhancer (Geyer and Corces 1992; Kellum and Schedl 1992). The chromatin barrier insulators function by preventing the advance of nearby condensed chromatin which would lead to a transcriptionally active chromatin region turning into a transcriptionally inactive chromatin region and resulting in silencing of gene expression. Insulators which inhibit the spread of heterochromatin, and thus gene silencing, recruit enzymes involved in histone modifications to prevent this process (Yang J, Corces V G. 2011; 110:43-76; Huang, Li et al. 2007; Dhillon, Raab et al. 2009). An insulator can have one or both of these functions and the chicken β-globin insulator (cHS4) is one such example. This insulator is the most extensively studied vertebrate insulator, is highly rich in G+C and has both enhancer-blocking and heterochromatic barrier functions (Chung J H, Whitely M, Felsenfeld G. Cell. 1993; 74:505-514). Other such insulators with enhancer blocking functions are not limited to but include the following: human β-globin insulator 5 (HS5), human β-globin insulator 1 (HS1), and chicken β-globin insulator (cHS3) (Farrell CM1, West A G, Felsenfeld G., Mol Cell Biol. 2002 June; 22(11):3820-31; J Ellis et al. EMBO J. 1996 Feb. 1; 15(3): 562-568). In addition to reducing unwanted distal interactions the insulators also help to prevent promoter interference (i.e. where the promoter from one transcription unit impairs expression of an adjacent transcription unit) between adjacent retroviral nucleic acid sequences. If the insulators are used between each of the retroviral vector nucleic acid sequences, then the reduction of direct read-through will help prevent the formation of replication-competent retroviral vector particles.

The insulator may be present between each of the retroviral nucleic acid sequences. In one embodiment, the use of insulators prevents promoter-enhancer interactions from one NOI expression cassette interacting with another NOI expression cassette in a nucleotide sequence encoding vector components.

An insulator may be present between the vector genome and gag-pol sequences. This therefore limits the likelihood of the production of a replication-competent retroviral vector and ‘wild-type’ like RNA transcripts, improving the safety profile of the construct. The use of insulator elements to improve the expression of stably integrated multigene vectors is cited in Moriarity et al, Nucleic Acids Res. 2013 April; 41(8):e92.

### Vector Titre

The skilled person will understand that there are a number of different methods of determining the titre of lentiviral vectors. Titre is often described as transducing units/mL (TU/mL). Titre may be increased by increasing the number of vector particles and by increasing the specific activity of a vector preparation.

### Therapeutic Use

The lentiviral vector as described herein or a cell or tissue transduced with the lentiviral vector as described herein may be used in medicine.

In addition, the lentiviral vector as described herein, a production cell of the invention or a cell or tissue transduced with the lentiviral vector as described herein may be used for the preparation of a medicament to deliver a nucleotide of interest to a target site in need of the same. Such uses of the lentiviral vector or transduced cell of the invention may be for therapeutic or diagnostic purposes, as described previously.

Accordingly, there is provided a cell transduced by the lentiviral vector as described herein.

A “cell transduced by a viral vector particle” is to be understood as a cell, in particular a target cell, into which the nucleic acid carried by the viral vector particle has been transferred.

In a preferred embodiment, the nucleotide of interest gives rise to a therapeutic effect.

“Target cell” is to be understood as a cell in which it is desired to express the NOI. The NOI may be introduced into the target cell using a viral vector of the present invention. Delivery to the target cell may be performed in vivo, ex vivo or in vitro.

The NOI may have a therapeutic or diagnostic application. Suitable NOIs include, but are not limited to sequences encoding enzymes, co-factors, cytokines, chemokines, hormones, antibodies, anti-oxidant molecules, engineered immunoglobulin-like molecules, single chain antibodies, fusion proteins, immune co-stimulatory molecules, immunomodulatory molecules, chimeric antigen receptors a transdomain negative mutant of a target protein, toxins, conditional toxins, antigens, transcription factors, structural proteins, reporter proteins, subcellular localization signals, tumour suppressor proteins, growth factors, membrane proteins, receptors, vasoactive proteins and peptides, anti-viral proteins and ribozymes, and derivatives thereof (such as derivatives with an associated reporter group). The NOIs may also encode micro-RNA. Without wishing to be bound by theory, it is believed that the processing of micro-RNA will be inhibited by TRAP.

In one embodiment, the NOI may be useful in the treatment of a neurodegenerative disorder.

In another embodiment, the NOI may be useful in the treatment of Parkinson's disease.

In another embodiment, the NOI may encode an enzyme or enzymes involved in dopamine synthesis. For example, the enzyme may be one or more of the following: tyrosine hydroxylase, GTP-cyclohydrolase I and/or aromatic amino acid dopa decarboxylase. The sequences of all three genes are available (GenBank® Accession Nos. X05290, U19523 and M76180, respectively).

In another embodiment, the NOI may encode the vesicular monoamine transporter 2 (VMAT2). In an alternative embodiment the viral genome may comprise a NOI encoding aromatic amino acid dopa decarboxylase and a NOI encoding VMAT2. Such a genome may be used in the treatment of Parkinson's disease, in particular in conjunction with peripheral administration of L-DOPA.

In another embodiment the NOI may encode a therapeutic protein or combination of therapeutic proteins.

In another embodiment, the NOI may encode a protein or proteins selected from the group consisting of glial cell derived neurotophic factor (GDNF), brain derived neurotrophic factor (BDNF), ciliary neurotrophic factor (CNTF), neurotrophin-3 (NT-3), acidic fibroblast growth factor (aFGF), basic fibroblast growth factor (bFGF), interleukin-1 beta (IL-1β), tumor necrosis factor alpha (TNF-α), insulin growth factor-2, VEGF-A, VEGF-B, VEGF-C/VEGF-2, VEGF-D, VEGF-E, PDGF-A, PDGF-B, hetero- and homo-dimers of PDFG-A and PDFG-B.

In another embodiment, the NOI may encode an anti-angiogenic protein or anti-angiogenic proteins selected from the group consisting of angiostatin, endostatin, platelet factor 4, pigment epithelium derived factor (PEDF), placental growth factor, restin, interferon-α, interferon-inducible protein, gro-beta and tubedown-1, interleukin(IL)-1, IL-12, retinoic acid, anti-VEGF antibodies or fragments/variants thereof such as aflibercept, thrombospondin, VEGF receptor proteins such as those described in U.S. Pat. Nos. 5,952,199 and 6,100,071, and anti-VEGF receptor antibodies.

In another embodiment, the NOI may encode anti-inflammatory proteins, antibodies or fragment/variants of proteins or antibodies selected from the group consisting of NF-kB inhibitors, IL1beta inhibitors, TGFbeta inhibitors, IL-6 inhibitors, IL-23 inhibitors, IL-18 inhibitors, Tumour necrosis factor alpha and Tumour necrosis factor beta, Lymphotoxin alpha and Lymphotoxin beta, LIGHT inhibitors, alpha synuclein inhibitors, Tau inhibitors, beta amyloid inhibitors, IL-17 inhibitors,

In another embodiment the NOI may encode cystic fibrosis transmembrane conductance regulator (CFTR).

In another embodiment the NOI may encode a protein normally expressed in an ocular cell.

In another embodiment, the NOI may encode a protein normally expressed in a photoreceptor cell and/or retinal pigment epithelium cell.

In another embodiment, the NOI may encode a protein selected from the group comprising RPE65, arylhydrocarbon-interacting receptor protein like 1 (AIPL1), CRB1, lecithin retinal acetyltransferace (LRAT), photoreceptor-specific homeo box (CRX), retinal guanylate cyclise (GUCY2D), RPGR interacting protein 1 (RPGRIP1), LCA2, LCA3, LCA5, dystrophin, PRPH2, CNTF, ABCR/ABCA4, EMP1, TIMP3, MERTK, ELOVL4, MYO7A, USH2A, VMD2, RLBP1, COX-2, FPR, harmonin, Rab escort protein 1, CNGB2, CNGA3, CEP 290, RPGR, RS1, RP1, PRELP, glutathione pathway enzymes and opticin.

In other embodiments, the NOI may encode the human clotting Factor VIII or Factor IX.

In other embodiments, the NOI may encode protein or proteins involved in metabolism selected from the group comprising phenylalanine hydroxylase (PAH), Methylmalonyl CoA mutase, Propionyl CoA carboxylase, Isovaleryl CoA dehydrogenase, Branched chain ketoacid dehydrogenase complex, Glutaryl CoA dehydrogenase, Acetyl CoA carboxylase, propionyl CoA carboxylase, 3 methyl crotonyl CoA carboxylase, pyruvate carboxylase, carbamoyl-phophate synthase ammonia, ornithine transcarbamylase, glucosylceramidase beta, alpha galactosidase A, glucosylceramidase beta, cystinosin, glucosamine(N-acetyl)-6-sulfatase, N-acetyl-alpha-glucosaminidase, N-sulfoglucosamine sulfohydrolase, Galactosamine-6 sulfatase, arylsulfatase A, cytochrome B-245 beta, ABCD1, ornithine carbamoyltransferase, argininosuccinate synthase, argininosuccinate lysase, arginase 1, alanine glycoxhylate amino transferase, ATP-binding cassette, sub-family B members.

In other embodiments, the NOI may encode a chimeric antigen receptor (CAR) or a T cell receptor (TCR). In one embodiment, the CAR is an anti-5T4 CAR. In other embodiments, the NOI may encode B-cell maturation antigen (BCMA), CD19, CD22, CD20, CD47, CD138, CD30, CD33, CD123, CD70, prostate specific membrane antigen (PSMA), Lewis Y antigen (LeY), Tyrosine-protein kinase transmembrane receptor (ROR1), Mucin 1, cell surface associated (Muc1), Epithelial cell adhesion molecule (EpCAM), endothelial growth factor receptor (EGFR), insulin, protein tyrosine phosphatase, non-receptor type 22, interleukin 2 receptor alpha, interferon induced with helicase C domain 1, human epidermal growth factor receptor (HER2), glypican 3 (GPC3), disialoganglioside (GD2), mesiothelin, vesicular endothelial growth factor receptor 2 (VEGFR2).

In one embodiment, the NOI encodes BCMA.

In one embodiment, the NOI encodes CD19.

In one embodiment, the NOI encodes CD22.

In one embodiment, the NOI encodes CD20.

In one embodiment, the NOI encodes CD47.

In one embodiment, the NOI encodes CD138.

In one embodiment, the NOI encodes CD30.

In one embodiment, the NOI encodes CD33.

In one embodiment, the NOI encodes CD123.

In one embodiment, the NOI encodes CD70.

In one embodiment, the NOI encodes PSMA.

In one embodiment, the NOI encodes LeY.

In one embodiment, the NOI encodes ROR1.

In one embodiment, the NOI encodes Mucin 1.

In one embodiment, the NOI encodes Muc1.

In one embodiment, the NOI encodes EpCAM.

In one embodiment, the NOI encodes EGFR.

In one embodiment, the NOI encodes insulin.

In one embodiment, the NOI encodes protein tyrosine phosphatase.

In one embodiment, the NOI encodes non-receptor type 22.

In one embodiment, the NOI encodes interleukin 2 receptor alpha.

In one embodiment, the NOI encodes interferon induced with helicase C domain 1.

In one embodiment, the NOI encodes HER2.

In one embodiment, the NOI encodes GPC3.

In one embodiment, the NOI encodes GD2.

In one embodiment, the NOI encodes mesiothelin.

In one embodiment, the NOI encodes VEGFR2.

In other embodiments, the NOI may encode a chimeric antigen receptor (CAR) against NKG2D ligands selected from the group comprising ULBP1, 2 and 3, H60, Rae-1a, b, g, d, MICA, MICB.

In further embodiments the NOI may encode SGSH, SUMF1, GAA, the common gamma chain (CD132), adenosine deaminase, WAS protein, globins, alpha galactosidase A, δ-aminolevulinate (ALA) synthase, δ-aminolevulinate dehydratase (ALAD), Hydroxymethylbilane (HMB) synthase, Uroporphyrinogen (URO) synthase, Uroporphyrinogen (URO) decarboxylase, Coproporphyrinogen (COPRO) oxidase, Protoporphyrinogen (PROTO) oxidase, Ferrochelatase, α-L-iduronidase, Iduronate sulfatase, Heparan sulfamidase, N-acetylglucosaminidase, Heparan-α-glucosaminide N-acetyltransferase, 3 N-acetylglucosamine 6-sulfatase, Galactose-6-sulfate sulfatase, β-galactosidase, N-acetylgalactosamine-4-sulfatase, β-glucuronidase and Hyaluronidase.

In addition to the NOI the vector may also comprise or encode a siRNA, shRNA, or regulated shRNA. (Dickins et al. (2005) Nature Genetics 37: 1289-1295, Silva et al. (2005) Nature Genetics 37:1281-1288).

### Indications

The vectors, including retroviral and AAV vectors, according to the present invention may be used to deliver one or more NOI(s) useful in the treatment of the disorders listed in WO 1998/05635, WO 1998/07859, WO 1998/09985. The nucleotide of interest may be DNA or RNA. Examples of such diseases are given below:
A disorder which responds to cytokine and cell proliferation/differentiation activity; immunosuppressant or immunostimulant activity (e.g. for treating immune deficiency, including infection with human immunodeficiency virus, regulation of lymphocyte growth; treating cancer and many autoimmune diseases, and to prevent transplant rejection or induce tumour immunity); regulation of haematopoiesis (e.g. treatment of myeloid or lymphoid diseases); promoting growth of bone, cartilage, tendon, ligament and nerve tissue (e.g. for healing wounds, treatment of burns, ulcers and periodontal disease and neurodegeneration); inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating, for example, septic shock or Crohn's disease); macrophage inhibitory and/or T cell inhibitory activity and thus, anti-inflammatory activity; anti-immune activity (i.e. inhibitory effects against a cellular and/or humoral immune response, including a response not associated with inflammation); inhibition of the ability of macrophages and T cells to adhere to extracellular matrix components and fibronectin, as well as up-regulated fas receptor expression in T cells.
Malignancy disorders, including cancer, leukaemia, benign and malignant tumour growth, invasion and spread, angiogenesis, metastases, ascites and malignant pleural effusion.
Autoimmune diseases including arthritis, including rheumatoid arthritis, hypersensitivity, allergic reactions, asthma, systemic lupus erythematosus, collagen diseases and other diseases.
Vascular diseases including arteriosclerosis, atherosclerotic heart disease, reperfusion injury, cardiac arrest, myocardial infarction, vascular inflammatory disorders, respiratory distress syndrome, cardiovascular effects, peripheral vascular disease, migraine and aspirin-dependent anti-thrombosis, stroke, cerebral ischaemia, ischaemic heart disease or other diseases.
Diseases of the gastrointestinal tract including peptic ulcer, ulcerative colitis, Crohn's disease and other diseases.
Hepatic diseases including hepatic fibrosis, liver cirrhosis.
Inherited metabolic disorders including phenylketonuria PKU, Wilson disease, organic acidemias, urea cycle disorders, cholestasis, and other diseases.
Renal and urologic diseases including thyroiditis or other glandular diseases, glomerulonephritis or other diseases.
Ear, nose and throat disorders including otitis or other oto-rhino-laryngological diseases, dermatitis or other dermal diseases.
Dental and oral disorders including periodontal diseases, periodontitis, gingivitis or other dental/oral diseases.
Testicular diseases including orchitis or epididimo-orchitis, infertility, orchidal trauma or other testicular diseases.
Gynaecological diseases including placental dysfunction, placental insufficiency, habitual abortion, eclampsia, pre-eclampsia, endometriosis and other gynaecological diseases.
Ophthalmologic disorders such as Leber Congenital Amaurosis (LCA) including LCA10, posterior uveitis, intermediate uveitis, anterior uveitis, conjunctivitis, chorioretinitis, uveoretinitis, optic neuritis, glaucoma, including open angle glaucoma and juvenile congenital glaucoma, intraocular inflammation, e.g. retinitis or cystoid macular oedema, sympathetic ophthalmia, scleritis, retinitis pigmentosa, macular degeneration including age related macular degeneration (AMD) and juvenile macular degeneration including Best Disease, Best vitelliform macular degeneration, Stargardt's Disease, Usher's syndrome, Doyne's honeycomb retinal dystrophy, Sorby's Macular Dystrophy, Juvenile retinoschisis, Cone-Rod Dystrophy, Corneal Dystrophy, Fuch's Dystrophy, Leber's congenital amaurosis, Leber's hereditary optic neuropathy (LHON), Adie syndrome, Oguchi disease, degenerative fondus disease, ocular trauma, ocular inflammation caused by infection, proliferative vitreo-retinopathies, acute ischaemic optic neuropathy, excessive scarring, e.g. following glaucoma filtration operation, reaction against ocular implants, corneal transplant graft rejection, and other ophthalmic diseases, such as diabetic macular oedema, retinal vein occlusion, RLBP1-associated retinal dystrophy, choroideremia and achromatopsia.
Neurological and neurodegenerative disorders including Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, strokes, post-polio syndrome, psychiatric disorders, myelitis, encephalitis, subacute sclerosing pan-encephalitis, encephalomyelitis, acute neuropathy, subacute neuropathy, chronic neuropathy, Fabry disease, Gaucher disease, Cystinosis, Pompe disease, metachromatic leukodystrophy, Wiscott Aldrich Syndrome, adrenoleukodystrophy, beta-thalassemia, sickle cell disease, Guillaim-Barre syndrome, Sydenham chorea, myasthenia gravis, pseudo-tumour cerebri, Down's Syndrome, Huntington's disease, CNS compression or CNS trauma or infections of the CNS, muscular atrophies and dystrophies, diseases, conditions or disorders of the central and peripheral nervous systems, motor neuron disease including amyotropic lateral sclerosis, spinal muscular atropy, spinal cord and avulsion injury.
Other diseases and conditions such as cystic fibrosis, mucopolysaccharidosis including Sanfilipo syndrome A, Sanfilipo syndrome B, Sanfilipo syndrome C, Sanfilipo syndrome D, Hunter syndrome, Hurler-Scheie syndrome, Morquio syndrome, ADA-SCID, X-linked SCID, X-linked chronic granulomatous disease, porphyria, haemophilia A, haemophilia B, post-traumatic inflammation, haemorrhage, coagulation and acute phase response, cachexia, anorexia, acute infection, septic shock, infectious diseases, diabetes mellitus, complications or side effects of surgery, bone marrow transplantation or other transplantation complications and/or side effects, complications and side effects of gene therapy, e.g. due to infection with a viral carrier, or AIDS, to suppress or inhibit a humoral and/or cellular immune response, for the prevention and/or treatment of graft rejection in cases of transplantation of natural or artificial cells, tissue and organs such as cornea, bone marrow, organs, lenses, pacemakers, natural or artificial skin tissue.
   siRNA, micro-RNA and shRNA

In certain other embodiments, the NOI comprises a micro-RNA. Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

In addition to the NOI the vector may also comprise or encode a siRNA, shRNA, or regulated shRNA (Dickins et al. (2005) Nature Genetics 37: 1289-1295, Silva et al. (2005) Nature Genetics 37:1281-1288).

Post-transcriptional gene silencing (PTGS) mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nature Medicine 11:429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA>30 bp has been found to activate the interferon response leading to shut-down of protein synthesis and non-specific mRNA degradation (Stark et al., Annu Rev Biochem 67:227-64 (1998)). However this response can be bypassed by using 21 nt siRNA duplexes (Elbashir et al., EMBO J. December 3; 20(23):6877-88 (2001), Hutvagner et al., Science. August 3, 293(5531):834-8. Eupub July 12 (2001)) allowing gene function to be analysed in cultured mammalian cells.

### Pharmaceutical Compositions

The present disclosure provides a pharmaceutical composition comprising the lentiviral vector as described herein or a cell or tissue transduced with the viral vector as described herein, in combination with a pharmaceutically acceptable carrier, diluent or excipient.

The present disclosure provides a pharmaceutical composition for treating an individual by gene therapy, wherein the composition comprises a therapeutically effective amount of a lentiviral vector. The pharmaceutical composition may be for human or animal usage.

The composition may comprise a pharmaceutically acceptable carrier, diluent, excipient or adjuvant. The choice of pharmaceutical carrier, excipient or diluent can be made with regard to the intended route of administration and standard pharmaceutical practice. The pharmaceutical compositions may comprise, or be in addition to, the carrier, excipient or diluent any suitable binder(s), lubricant(s), suspending agent(s), coating agent(s), solubilising agent(s) and other carrier agents that may aid or increase vector entry into the target site (such as for example a lipid delivery system).

Where appropriate, the composition can be administered by any one or more of inhalation; in the form of a suppository or pessary; topically in the form of a lotion, solution, cream, ointment or dusting powder; by use of a skin patch; orally in the form of tablets containing excipients such as starch or lactose, or in capsules or ovules either alone or in admixture with excipients, or in the form of elixirs, solutions or suspensions containing flavouring or colouring agents; or they can be injected parenterally, for example intracavernosally, intravenously, intramuscularly, intracranially, intraoccularly intraperitoneally, or subcutaneously. For parenteral administration, the compositions may be best used in the form of a sterile aqueous solution which may contain other substances, for example enough salts or monosaccharides to make the solution isotonic with blood. For buccal or sublingual administration, the compositions may be administered in the form of tablets or lozenges which can be formulated in a conventional manner.

The lentiviral vector as described herein may also be used to transduce target cells or target tissue ex vivo prior to transfer of said target cell or tissue into a patient in need of the same. An example of such cell may be autologous T cells and an example of such tissue may be a donor cornea.

### Variants, Derivatives, Analogues, Homologues and Fragments

In addition to the specific proteins and nucleotides mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence retains the required activity or ability. Amino acid substitutions may include the use of non-naturally occurring analogues.

Proteins used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R H |
| AROMATIC | | F W Y |

The term “homologue” means an entity having a certain homology with the wild type amino acid sequence and the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95%, 97 or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95%, 97%, 98% or 99% identical to the subject sequence. Although homology can also be considered in terms of similarity, in the context of the present invention it is preferred to express homology in terms of sequence identity.

Homology comparisons can be conducted by eye, or more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Research 12:387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol Lett (1999) 174(2):247-50; FEMS Microbiol Lett (1999) 177(1):187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software usually does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the break. The DNA is then expressed in accordance with the invention to make the encoded protein. These methods are only illustrative of the numerous standard techniques known in the art for manipulation of DNA sequences and other known techniques may also be used.

All variants, fragments or homologues of the regulatory protein suitable for use in the cells and/or modular constructs of the invention will retain the ability to bind the cognate binding site of the NOI such that translation of the NOI is repressed or prevented in a viral vector production cell.

All variants fragments or homologues of the binding site will retain the ability to bind the cognate RNA-binding protein, such that translation of the NOI is repressed or prevented in a viral vector production cell.

### Codon Optimisation

The polynucleotides used in the present invention (including the NOI and/or components of the vector production system) may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including retroviruses, use a large number of rare codons and changing these to correspond to commonly used mammalian codons, increases expression of a gene of interest, e.g. a NOI or packaging components in mammalian production cells, can be achieved. Codon usage tables are known in the art for mammalian cells, as well as for a variety of other organisms.

Codon optimisation of viral vector components has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. In lentiviral vectors codon optimisation also overcomes the Rev/RRE requirement for export, rendering optimised sequences Rev-independent. Codon optimisation also reduces homologous recombination between different constructs within the vector system (for example between the regions of overlap in the gag-pol and env open reading frames). The overall effect of codon optimisation is therefore a notable increase in viral titre and improved safety.

In one embodiment only codons relating to INS are codon optimised. However, in a much more preferred and practical embodiment, the sequences are codon optimised in their entirety, with some exceptions, for example the sequence encompassing the frameshift site of gag-pol (see below).

The gag-pol gene of lentiviral vectors comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimised. Retaining this fragment will enable more efficient expression of the Gag-Pol proteins. For EIAV the beginning of the overlap has been taken to be nt 1262 (where nucleotide 1 is the A of the gag ATG) and the end of the overlap to be nt 1461. In order to ensure that the frameshift site and the gag-pol overlap are preserved, the wild type sequence has been retained from nt 1156 to 1465.

Derivations from optimal codon usage may be made, for example, in order to accommodate convenient restriction sites, and conservative amino acid changes may be introduced into the Gag-Pol proteins.

In one embodiment, codon optimisation is based on lightly expressed mammalian genes. The third and sometimes the second and third base may be changed.

Due to the degenerate nature of the genetic code, it will be appreciated that numerous gag-pol sequences can be achieved by a skilled worker. Also there are many retroviral variants described which can be used as a starting point for generating a codon-optimised gag-pol sequence. Lentiviral genomes can be quite variable. For example there are many quasi-species of HIV-1 which are still functional. This is also the case for EIAV. These variants may be used to enhance particular parts of the transduction process. Examples of HIV-1 variants may be found at the HIV Databases operated by Los Alamos National Security, LLC at http://hiv-web.lanl.gov. Details of EIAV clones may be found at the National Center for Biotechnology Information (NCBI) database located at http://www.ncbi.nlm.nih.gov.

The strategy for codon-optimised gag-pol sequences can be used in relation to any retrovirus. This would apply to all lentiviruses, including EIAV, FIV, BIV, CAEV, VMR, SIV, HIV-1 and HIV-2. In addition this method could be used to increase expression of genes from HTLV-1, HTLV-2, HFV, HSRV and human endogenous retroviruses (HERV), MLV and other retroviruses.

Codon optimisation can render gag-pol expression Rev-independent. In order to enable the use of anti-rev or RRE factors in the lentiviral vector, however, it would be necessary to render the viral vector generation system totally Rev/RRE-independent. Thus, the genome also needs to be modified. This is achieved by optimising vector genome components.

Advantageously, these modifications also lead to the production of a safer system absent of all additional proteins both in the producer and in the transduced cell.

It is demonstrated in the present Examples that the modified U1 and associated methods and uses of the invention as described herein may advantageously result in acceptable or advantageous safety profiles.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### General Molecular/Cell Biology Techniques and Assays

Modified U1 snRNA Expression Constructs

The DNA-based expression constructs for the modified U1 snRNAs comprise the conserved sequences in the endogenous U1 snRNA gene driving RNA transcription and termination, highlighted below in the non-limiting example of the 256U1 (also referred to as U1_256) snRNA:

(SEQ ID NO: 15)
TAAGGACCAGCTTCTTTGGGAGAGAACAGACGCAGGGGCGGGAGGGAAA
AAGGGAGAGGCAGACGTCACTTCCCCTTGGCGGCTCTGGCAGCAGATTG
GTCGGTTGAGTGGCAGAAAGGCAGACGGGGACTGGGCAAGGCACTGTCG
GTGACATCACGGACAGGGCGACTTCTATGTAGATGAGGCAGCGCAGAGG
CTGCTGCTTCGCCACTTGCTGCTTCACCACGAAGGAGTTCCCGTGCCCT
GGGAGCGGGTTCAGGACCGCTGATCGGAAGTGAGAATCCCAGCTGTGTG
TCAGGGCTGGAAAGGGCTCGGGAGTGCGCGGGGCAAGTGACCGTGTGTG
TAAAGAGTGAGGCGTATGAGGCTGTGTCGGGGCAGAGGCCCAAGATCTC
atttgccgtgcgcgcttGCAGGGGAGATACCATGATCACGAAGGTGGTT
TTCCCAGGGCGAGGCTTATCCATTGCACTCCGGATGTGCTGACCCCTGC
GATTTCCCCAAATGTGGGAAACTCGACTGCATAATTTGTGGTAGTGGGG
GACTGCGTTCGCGCTTTCCCCTGGTTTCAAAAGTAGACTGTACGCTAAG
GGTCATATCTTTTTTTGTTTTGGTTTGTGTCTTGGTTGGCGTCTTAAAT
GTTAA
Key:
Upper case only = U1 PolII promoter (nt 1-392); lower case = retargeting region (nt 393-409); lower case bold = retargeting sequence [in this example targeting nt256-270 of wild type HIV-1 packaging signal] (nt395-409); upper case italics = main U1 snRNA sequence [clover-leaf] (nt 410-562); upper case underlined = transcription termination region (nt 563-652)

A summary of the initial modified U1 snRNAs and controls used in the study is presented in the table below, indicating the new annealing sequence and the target site sequence (sequences are represented in the 5′ to 3′ direction).

TABLE 1
A list of sequences describing the target-annealing sequences (heterologous
sequence that is complementary to the target sequence) within test modified U1 snRNAse
and control U1 snRNAs, and their target sequences used in the initial study.
Nucleotides are presented as DNA as they would be encoded within their respective
expression cassettes at the ‘retargeting region’. The (AT) motif was present in all
initial constructs, which forms the first two nucleotides of the U1 snRNA molecule
in each case. The target sequence numbers refer to targets in the NL4-3 (GenBank:
M19921.2) or HXB2 (GenBank: K03455.1) strains of HIV-1 where denoted, since the
lentiviral vector genome in this study contained a hybrid packaging signal composed
of these two highly conserved strains (packaging sequence used in this study is most
similar to the vector sequence in GenBank: MH782475.1)

| Modified U1 | | U1 snRNA target-annealing |
|---|---|---|
| snRNA* | HIV-1 target sequence [NL4-3]** | sequence |
| U1_16 | 16-GACCAGATCTGAGCC-30 | (AT)GGCTCAGATCTGGTC |
| | (SEQ ID NO: 16) | (SEQ ID NO: 17) |
| U1_31 | 31-TGGGAGCTCTCTGGC-45 | (AT)GCCAGAGAGCTCCCA |
| | (SEQ ID NO: 18) | (SEQ ID NO: 19) |
| U1_76 | 76-TAAAGCTTGCCTTGA-90 | (AT)TCAAGGCAAGCTTTA |
| | (SEQ ID NO: 20) | (SEQ ID NO: 21) |
| U1_136 | 136-TAGAGATCCCTCAGA-150 | (AT)TCTGAGGGATCTCTA |
| | (SEQ ID NO: 22) | (SEQ ID NO: 23) |
| U1_179(9 nt) | 179-GCAGTGGCG-187 | (AT)CGCCACTGC |
| | (SEQ ID NO: 24) | (SEQ ID NO: 25) |
| U1_181 | 181-AGTGGCGCCCGAACA-195 | (AT)TGTTCGGGCGCCACT |
| | (SEQ ID NO: 26) | (SEQ ID NO: 27) |
| U1_196 | 196-GGGACTTGAAAGCGA-210 | (AT)TCGCTTTCAAGTCCC |
| | (SEQ ID NO: 28) | (SEQ ID NO: 29) |
| U1_211 | 211-AAGggAAaCCAGAGG-225 | (AT)CCTCTGGTTTCCCTT (SEQ |
| | (SEQ ID NO: 30) | ID NO: 31) |
| U1_226 | 226-AGcTCTCTCGACGCA-240 | (AT)TGCGTCGAGAGAGCT |
| | (SEQ ID NO: 73) | (SEQ ID NO: 74) |
| U1_241 | 241-GGACTCGGCTTGCTG-255 | (AT)CAGCAAGCCGAGTCC (SEQ |
| | (SEQ ID NO: 32) | ID NO: 33) |
| U1_256 | 256-AAGCGCGCACGGCAA-270 | (AT)TTGCCGTGCGCGCTT (SEQ |
| | (SEQ ID NO: 34) | ID NO: 35) |
| U1_271 | 271-GAGGCGAGGGGCGGC-285 | (AT)GCCGCCCCTCGCCTC (SEQ |
| | (SEQ ID NO: 36) | ID NO: 37) |
| U1_286 | 286-GACTGGTGAGTACGC-300 | (AT)GCGTACTCACCAGTC (SEQ |
| | (SEQ ID NO: 38) | ID NO: 39) |
| U1_305(9 nt) | 305-AATTTTGAC(TA)-313/5 | (AT)GTCAAAATT (SEQ ID |
| | (SEQ ID NO: 40) | NO: 41) |
| U1_305 | 305-AATTTTGACTAGCGG-319 | (AT)CCGCTAGTCAAAATT (SEQ |
| | (SEQ ID NO: 42) | ID NO: 43) |
| U1_316 | 316-GCGGAGGCTAGAAGG-330 | (AT)CCTTCTAGCCTCCGC (SEQ |
| | (SEQ ID NO: 44) | ID NO: 45) |
| U1_331 | 331-AGAGAGATGGGTGCG-345 | (AT)CGCACCCATCTCTCT (SEQ |
| | (SEQ ID NO: 46) | ID NO: 47) |
| U1_346 | 346-AGAGCGTCgGTATTA-360 | (AT)TAATACTGACGCTCT (SEQ |
| | (SEQ ID NO: 48) | ID NO: 49) |
| U1_361 | 361-AGCGGGGGAGAATTA-375 | (AT)TAATTCTCCCCCGCT (SEQ |
| | (SEQ ID NO: 50) | ID NO: 51 |
| U1_376 | 376-GATCGCGATGGGAAA-390 | (AT)TTTCCCATCGCGATC (SEQ |
| | (SEQ ID NO: 52) | ID NO: 53) |
| U1_391 | 389-AAATTCGGTTAAGGC-403 | (AT)GCCTTAACCGAATTT (SEQ |
| | (SEQ ID NO: 54) | ID NO: 55) |
| U1_690 | 7159-GATCTTCAGACCTGG-7173 | (AT)CCAGGTCTGAAGATC (SEQ |
| | (SEQ ID NO: 56) | ID NO: 57) |
| U1_1203 | 7672-TTACACAAGCTTAAT-7686 | (AT)ATTAAGCTTGTGTAA (SEQ |
| | (SEQ ID NO: 58) | ID NO: 59) |
| U1_1546 | 4375-TAGTAGACATAATAG-4389 | (AT)CTATTATGTCTACTA (SEQ |
| | (SEQ ID NO: 60) | ID NO: 61) |
| Control | | U1 snRNA target-annealing |
| U1 snRNA | Target sequence | sequence |
| U1_LacZ1 | 388-CTACAGGAA-396 (SEQ ID | (AT)TTCCTGTAG (SEQ ID |
| | NO: 62) | NO: 63) |
| U1_LacZ2 | 438-TCATCTGTG-446 (SEQ ID | (AT)CACAGATGA (SEQ ID |
| | NO: 64) | NO: 65) |

*numbering relative to vector genome RNA sequence
**lower case target sequence is for (HXB2), underlined target sequence is an AA > CGCG frameshift in the gag ORF (U1 376)

### Adherent Cell Culture, Transfection and Lentiviral Vector Production

HEK293T cells were maintained in complete media (Dulbecco's Modified Eagle Medium (DMEM) (Sigma) supplemented with 10% heat-inactivated (FBS)(Gibco), 2 mM L-glutamine (Sigma) and 1% non-essential amino acids (NEAA) (Sigma)), at 37° C. in 5% CO₂.

The standard scale production of HIV-1 vectors in adherent mode was in 10 cm dishes under the following conditions (all conditions were scaled by area when performed in other formats): HEK293T cells were seeded at 3.5×10⁵ cell per ml in 10 mL complete media and approximately 24 hours later the cells were transfected using the following mass ratios of plasmids per 10 cm plate: 4.5 μg Genome, 1.4 μg Gag-Pol, 1.1 μg Rev, 0.7 μg VSV-G and between 0.01 and 2 μg of modified U1 snRNA plasmid.

Transfection was mediated by mixing DNA with Lipofectamine 2000CD in Opti-MEM as per manufacturer's protocol (Life Technologies). Sodium butyrate (Sigma) was added ˜18 hours later to 10 mM final concentration for 5-6 h, before 10 ml fresh serum-free media replaced the transfection media. Typically, vector supernatant was harvested 20-24 hours later, and then filtered (0.22 μm) and frozen at −20/−80° C. As a positive control for nuclease treatment, typically Benzonase® was added to the harvests at 5U/mL for 1 hour prior to filtration.

### Suspension Cell Culture, Transfection and Lentiviral Vector Production

HEK293T.1-65s suspension cells were grown in Freestyle+0.1% CLC (Gibco) at 37° C. in 5% CO₂, in a shaking incubator (25 mm orbit set at 190 RPM). All vector production using suspension was carried out in 24-well plates (1 mL volumes, on a shaking platform), 25 mL shake flasks or in bioreactors (≤5 L). HEK293 Ts cells were seeded at 8×10⁵ cells per ml in serum-free media and were incubated at 37° C. in 5% CO₂, shaking, throughout vector production. Approximately 24 hours after seeding the cells were transfected using the following mass ratios of plasmids per effective final volume of culture at transfection: 0.95 μg/mL Genome, 0.1 μg/mL Gag-Pol, 0.6 μg/mL Rev, 0.7 μg/mL VSV-G, and between 0.01 to 0.2 μg/mL modified U1 snRNA plasmid.

Transfection was mediated by mixing DNA with Lipofectamine 2000CD in Opti-MEM as per manufacturer's protocol (Life Technologies). Sodium butyrate (Sigma) was added ˜18 hrs later to 10 mM final concentration. Typically, vector supernatant was harvested 20-24 hours later, and then filtered (0.22 μm) and frozen at −20/−80° C. As a positive control for nuclease treatment, typically Benzonase® was added to the harvests at 5U/mL for 1 hour prior to filtration.

### Lentiviral Vector Titration Assays

For lentiviral vector titration by GFP marker-containing cassette, HEK293T cells were seeded at 1.2×10⁴ cells/well in 96-well plates. GFP-encoding viral vectors were used to transduce the cells in complete media containing 8 mg/ml polybrene and 1×Penicillin Streptomycin for approximately 5-6 hours after which fresh media was added. The transduced cells were incubated for 2 days at 37° C. in 5% CO₂. Cultures were then prepared for flow cytometry using an Attune-NxT (Thermofisher). Percent GFP expression was measured and vector titres were calculated using a predicted cell count of 2×10⁴ cells at the time of transduction (base on typical growth rate), the dilution factor of the vector sample, the percentage positive GFP population and total volume at transduction.

### Lentiviral Vector Titration by Integration-Assay

For lentiviral vector titration by integration assay, 0.5 mL volumes of neat to 1:5 diluted vector supernatants were used to transduce 1×10⁵ HEK293T cells at 12-well scale in the presence of 8 μg/mL polybrene. Cultures were passaged for 10 days (1:5 splits every 2-3 days) before host DNA was extracted from 1×10⁶ cell pellets. Duplex quantitative PCR was carried out using a FAM primer/probe set to the HIV packaging signal (ψ) and to RRP1, and vector titres (TU/mL) calculated using the following factors: transduction volume, vector dilution, RRP1-normalized HIV-1 ψ copies detected per reaction.

### SDS-PAGE and Immunoblotting

Standard SDS-PAGE and Immunoblotting protocols were carried out primarily on End-of-vector production cells, post vector harvest. For immunoblotting analysis of vector particles, ˜2 mL of filtered vector supernatnant was centrifuged at 21,000 rpm in a microfuge for 1-2 hours at 4° C., before the ‘pellet’ was resuspended in 20-30 μL PBS. These concentrated vector preparations were quantified by PERT assay (below) and 7×10⁴ PERT-predicted TUs of vector loaded per well of a SDS-PAGE gel. Approximately 1×10⁶ end-of-vector production cell were lysed in 200 μL fractionation buffer and the nuclei removed by centrifugation. Protein samples were qualified by BioRad assay and typically 5 μg of protein was loaded onto pre-formed, 12-15 well, 4-20% acrylamide gels. Proteins were transferred onto nitrocellulose at 45V for 3 hours on ice. Blots were blocked in 5% milk PBS/tween-20 overnight at 4° C. Blots were probed with primary antibodies and HRP-secondary antibodies at typically 1:100 dilution in blocking buffer. Immunoblots were analysed by ECL-detection followed by X-ray film exposure.

### RNA Extraction and RT-qPCR Assays

Total RNA was extracted and purified from cells or LV samples using an RNeasy mini kit (QIAGEN). One microgram of RNA was treated with DNAse I (Ambion) for 1 hour before inactivation. 50 ng of DNAase I-treated RNA was used in qRT-PCR reactions comprised of Taqman® One step RT-PCR master mix (Life Technologies) under standard chemistry RT-PCR cycling conditions using a QuantStudio™ 6 (Life Technologies). Target specific primer/probe sets were employed. Negative control reactions contained no RT to control for DNA contamination.

### Analysis of Proteins by Mass Spectrometry

### Sample Preparation

Samples were prepared for MS analysis by in-solution tryptic digestion followed by peptide clean up. In brief, samples were denatured using a high molarity urea buffer prior the addition of dithiothreitol (DTT) and iodoacetamide as reducing and alkylating reagents respectively. After protein linearization, trypsin was added to each sample following dilution to reduce the concentration of urea. Protein digestion was completed by over-night incubation at 37° C. Trypsin activity was then neutralised by acidic pH by the addition of trifluoroacetic acid (TFA). The digested peptides were cleaned up and desalted by C18 columns. The columns were initially activated by acetonitrile (ACN) and thoroughly washed with a 0.1% TFA solution before loading the digested peptides. After additional washes with the 0.1% TFA solution, peptides were eluted by an acidified 70% ACN solution and collected in low-binding tubes. Next, ACN was removed by evaporation using a SpeedVac, and the cleaned, desalted dried peptides were resuspended in a solution containing 2% ACN and 0.1% Formic Acid (FA).

### Liquid Chromatography and Mass Spectrometry.

Peptides were analysed on an UltiMate 3000 (Thermo Fisher Scientific) coupled online to a Q Exactive™ HF mass spectrometer (Thermo Fisher Scientific). Peptides were loaded on a μPAC™ trapping column (PharmaFluidics, Ghent, Belgium) at a flow rate of 5 μL/min for 3 min and were subsequently separated on a 50 cm pPAC™ column (PharmaFluidics) using a 120 min nonlinear gradient of acetonitrile from 0.8% to 78% in 0.1% formic acid. Column temperature was kept at 50° C. using the UltiMate 3000 column oven. The Q Exactive™ HF was operated in a data independent (DIA) manner in the m/z range of 350-1,150. Full scan spectra were recorded with a resolution of 120,000 using an automatic gain control (AGC) target value of 3×106 with a maximum injection time of 20 ms. The Full scans were followed by 100 windows of 8.5 Th width using an overlap of 0.5 Th. DIA spectra were recorded at a resolution of 30 000 using an AGC target value of 2×105 with a maximum injection time of 60 ms and a fixed first mass of 200 Th. Normalized collision energy (NCE) was set to 28% and default charge state was set to 3. Peptides were ionized using An EASY-spray electrospray emitter (Thermo Fisher Scientific) at a spray voltage of 2.0 kV and a heated capillary temperature of 250° C.

### Data Analysis Using DIA-NN.

Protein sequences of Homo sapiens uniprot reference proteome were concatenated with lentiviral proteins (GAG, POL) and frequent contaminants to generate a predicted spectral library for this project. Spectral libraries were predicted for all possible peptides with strict trypsin specificity (KR not P) in the m/z range of 350-1,150 allowing up to one missed cleavage site. The mass spectra were analyzed in DIA-NN (Version 1.7) using fixed mass tolerances of 5 ppm for MS1 and 10 ppm for MS² spectra with enabled “RT profiling” using the “any LC” quantification strategy. The false discovery rate was set to 0.1% for precursor identifications and proteins were grouped according to their respective genes. Proteins were quantified using the “normalised.unique” intensities.

### Differential Expression Analysis

Differential expression analysis was conducted in R using the BioConductor DEP package. Proteins with missing values were initially filtered such that at least one condition had quantification for all replicates. Variance stabilization normalization was performed using the BioConductor VSN package. Missing values were then imputed using Quantile Regression Imputation of Left-Censored data (QRILC). Differential expression analysis was performed using protein-wise linear models with empirical Bayes statistics. P-values were adjusted for multiple testing using the Benjamini & Hochberg method.

### Generation of CAR-T Cells and Assessment of Functionality

### Cell Transduction

Peripheral blood mononuclear cells (PBMC) from three healthy human donors were purchased from a commercial supplier. 1.5×10⁶ PBMC per well were cultured with 4.5×10⁶ CD3/CD28 T Cell Expander beads in modified T cell medium, with 100 IU/mL recombinant human IL-2 (12 well plates). LV-CAR/LV-CAR[+256U1] were added to relevant wells at an estimated moiety of infection (MOI) of 1.25 or 0.3 as indicated. Cells were maintained at a concentration of 1.0×10⁵ viable cells per mL by increasing the volume of modified T cell medium with 100 IU/mL recombinant human IL-2 (cultured vessel size increased as required). After thirteen days in culture cells were frozen at a concentration of 1.0×10⁷ viable cells per mL in cell freezing medium. The percentage of cells transduced was measured by flow cytometry after 8 days in initial expansion, and 5 days after revival.

### Functionality Testing

Cell lines used to test CAR-T cell responses were SKOV-3 (an ovarian cancer cell line that expresses high levels of 5T41) and three acute myeloid leukaemia (AML) cell lines—THP-1, Kasumi-1, and AML-193, the latter a 5T4-negative cell line. Target cell lines were labelled with a fluorescent cell tracing dye to allow subsequent identification by flow cytometry. Approximately 1×10⁵ CAR-T cells were co-cultured in triplicate with 1×10⁵ of each cell line in 96-well round-bottom plates. After 24 hours a volume of culture supernatant was removed from each well for analysis of interferon gamma and granzyme B by cytometric bead array. After 40 hours cells were harvested and stained with a fluorescent viability dye. The percentage of non-viable target cells in each experimental well was measured by flow cytometry.

### Example 1

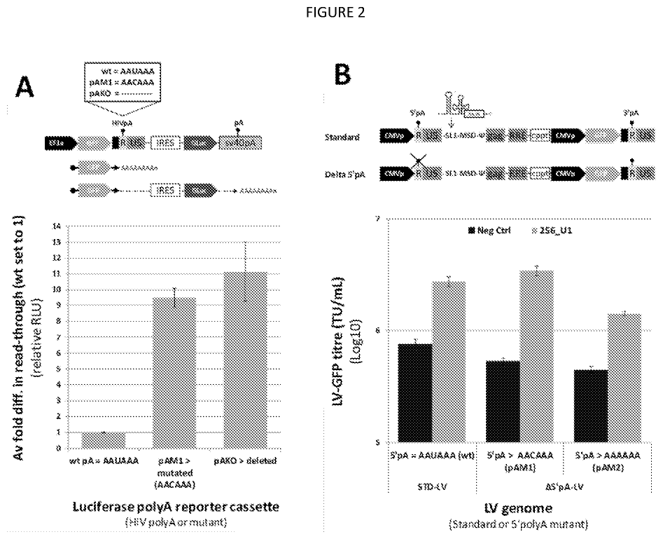

A GFP-polyA-GLuciferase reporter cassette was designed to assess the impact of polyA signal mutants on transcriptional read-through the HIV-1 polyA site. The reporter encodes an upstream GFP ORF (in order to be able to normalize for transfection efficiency), a standard 3′SIN-LTR sequence (comprising the RU5 sequence which harbors the HIV-1 polyA signal), followed by an IRES-Gluc sequence and SV40 polyA signal. Therefore, any read-through the HIV-1 polyA signal was measurable by luciferase activity, which was normalized by GFP expression. The impact of two polyA signal mutations (pAM1=AAUAAA>AACAAA; pAKO=deletion of AAUAAAA) and the wild type polyA signal (wt pA=AAUAAA) was measured (FIG. 2A).

A standard lentiviral vector genome, and two genomes containing different 5′LTR polyA signal mutants (pAM1 or pAM2), were used to make vector particles either in the absence or presence of a modified U1 snRNA (256_U1, supplied in parallel during production), and then titrated. Vectors produced in the presence of the modified U1 snRNA were 3-to-6 fold greater than without, and this was independent of whether a functional polyA site was present in the 5′LTR (FIG. 2B). This indicates that the modified U1 snRNA is not acting to suppress any leaky read-through of the polyA (i.e. the endogenous U1 snRNA is binding to the major splice donor site and fully suppressing pre-mature polyadenylation) and must therefore be acting to increase vector titres by some other novel mechanism (possibly by improving vRNA stability/nuclear export).

### Example 2

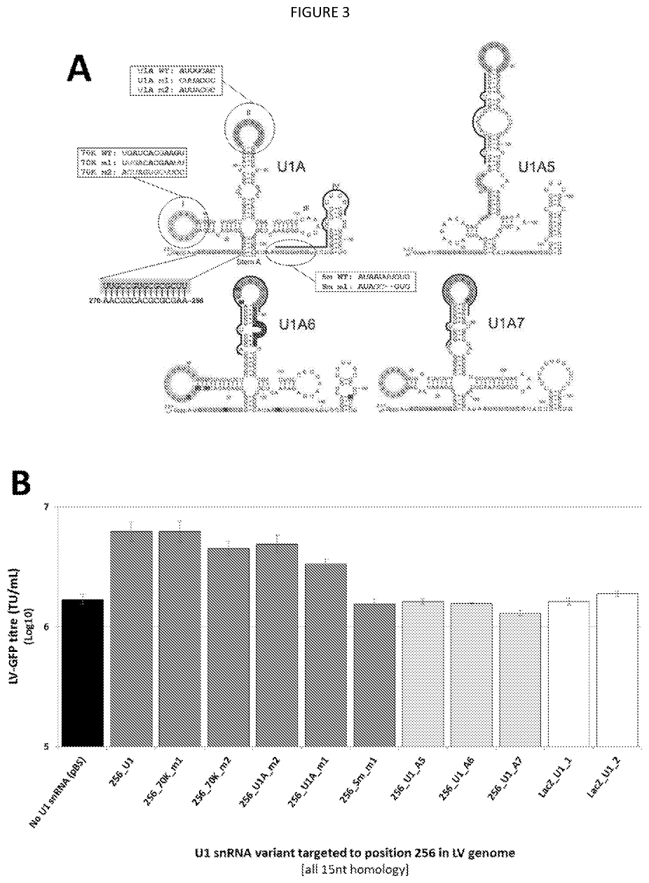

An experiment was performed to evaluate other features of the U1 snRNA molecule in their possible requirement in the invention. Several modified U1 snRNA expression cassettes were constructed—all having the target sequence to the ‘256’ location in the HIV-1 packaging region (FIG. 3A). Two variants contained published mutations within the stem loop I region, ablating U1-70K protein binding (256_70K_m1 and 256_70K_m2), two variants contained published mutations within the stem loop II region, ablating U1A protein binding (256_U1A_m1 and 256_U1A_m2), and one variant containing a mutated Sm-protein binding motif (256_SM_m1) (Alexander, M. R. et al., 2010, Nucleic Acids Res., 38: 3041-53; Ashe, M. P. et al., 2000, RNA, 6: 170-7). Three other cassettes expressing naturally occurring U1 snRNAs (U1A5, U1A6, and U1A7) were also constructed—these having conserved Sm binding regions but very different sequences within the cloverleaf structure (and therefore unlikely to bind U1-70K or U1A). Finally, two control U1 snRNA constructs targeting lacZ gene sequences were generated as negative controls.

When these modified U1 snRNAs were individually co-transfected with lentiviral vector components (marker gene=GFP) into HEK293T production cells, vector titres were enhanced by 2-4 fold by 256_U1 and the 70K or U1A protein binding mutant U1s but not the other variants (FIG. 3A). U1A-based snRNAs increased LV titres independently of functional U1A-70K and U1A binding loops but the titre boost was dependent on the Sm protein binding motif. snRNA variants U1A5, U1A6 and U1A7 did not increase LV titres indicating that some structural feature of U1A snRNA is required for the effect.

### Example 3

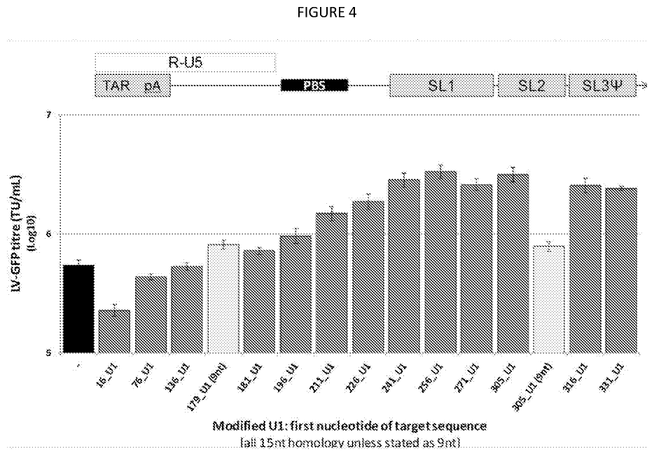

Experiment performed in adherent HEK293T vector production setting. A standard lentiviral vector encoding GFP was produced in the absence or the presence of modified U1 snRNAs with targeting sequence to sites along the length of the 5′end of the vector genome vRNA molecule comprising either 15 nucleotide or 9 nucleotide targeting lengths of complementarity. Modified U1 snRNAs are named according to the first nucleotide of the targeting sequence site along the length of the 5′end of the vector genome vRNA molecule (see Table I). The data indicates that the increase in magnitude of the boost in vector titre correlates with target sites that are further away from the 5′ polyA site, with an ideal target region encoded within the SL1 loop of the packaging signal (FIG. 4). The data also indicates that utilisation of targeting lengths of complementarity of 15 nucleotides instead of 9 nucleotides (as per endogenous U1 snRNA) produce a more robust increase in vector titres.

### Example 4

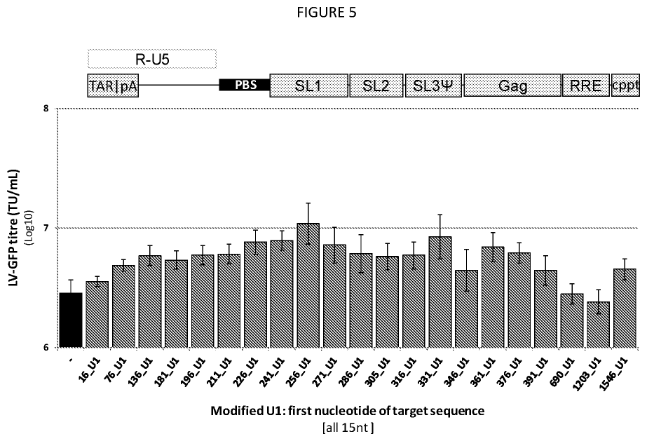

Experiment performed in suspension, serum-free HEK293T vector production setting. A standard lentiviral vector encoding GFP was produced in the absence or the presence of modified U1 snRNAs with targeting sequence to sites along the length of the 5′end of the vector genome vRNA molecule comprising 15 nucleotide targeting lengths of complementarity. Modified U1 snRNAs are named according to the first nucleotide of the targeting sequence site along the length of the 5′end of the vector genome vRNA molecule. The data indicates that the increase in magnitude of the boost in vector titre correlates with target sites that are further away from the 5′ polyA site, with an ideal target region encoded within the SL1 loop of the packaging signal (FIG. 5).

### Example 5

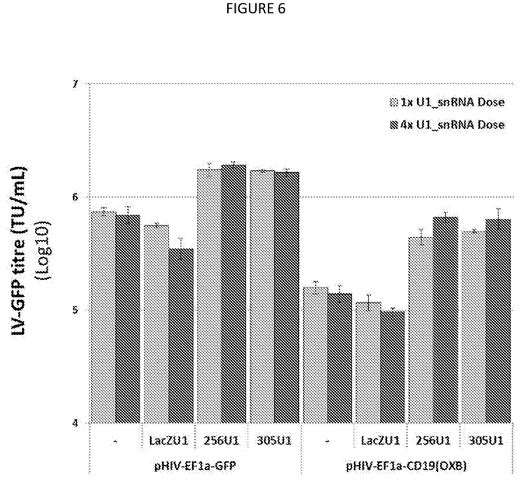

Experiment performed in adherent HEK293T vector production setting. Standard lentiviral vectors encoding GFP (pHIV-EF1a-GFP) or a chimeric antigen receptor to CD19* (pHIV-EF1a-CD19) were produced in the absence or the presence of modified U1 snRNAs targeting either sites within the lentiviral vector packaging region (256U1 or 305U1) or a LacZ control (LacZU1). Modified U1 snRNAs are named according to the first nucleotide of the targeting sequence site within the lentiviral vector packaging region (see Table I). The modified U1 snRNA expression constructs were supplied at two different doses: 1× and 4×. The data demonstrates that the invention may be applied independently of the payload of the vector genome (FIG. 6).

The CD19 CAR in this and all Other Examples Provided Herein Utilised a scFV Based on Publically Available Sequences:

Heavy chain-GenBank CAA67618.1:
(SEQ ID NO: 191)
QVQLQQSGAELVRPGSSVKISCKASGYAFSSYWMNWVKQRPGQGLEWIG
QIWPGDGDTNYNGKFKGKATLTADESSSTAYMQLSSLASEDSAVYFCAR
RETTTVGRYYYAMDYWGQGTSVTVSS
Light chain (kappa)-GenBank AAB34430.1:
(SEQ ID NO: 192)
ELVLTQSPASLAVSLGQRATISCKASQSVDYDGDSYLNWYQQIPGQPPK
LLIYDASNLVSGIPPRFSGSGSGTDFTLNIHPVEKVDAATYHCQQSTED
PWTFGGGTKLEIKRRS

### Example 6—Promiscuous Splicing from the MSD, Reduced Titres of MSD-2KO Lentiviral Vectors and Titre Recovery/Boost by Re-Directed U1 snRNAs

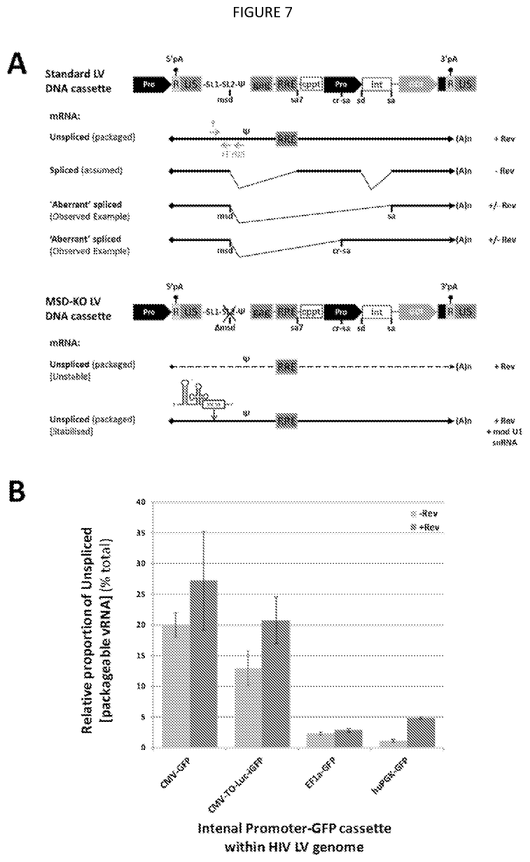

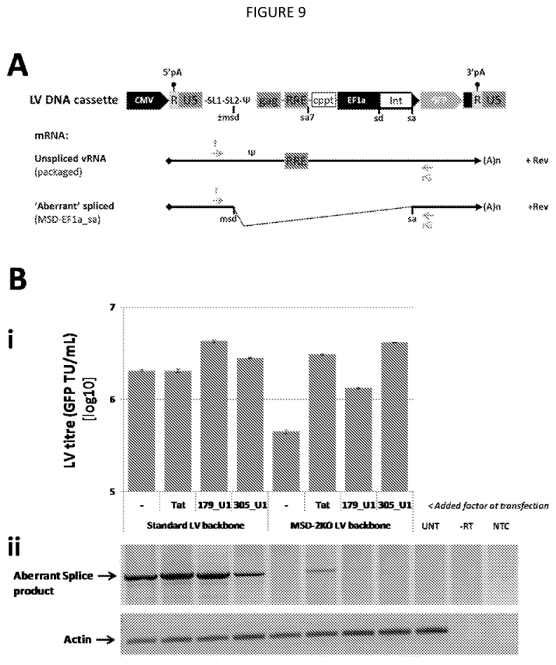

The general architecture of lentiviral vector genomes has remained consistent through all three generations of vector systems Toshie SAKUMA, Michael A. BARRY and Yasuhiro IKEDA. Biochem. J. (2012) 443, 603-618), with maintenance of the 5′region of the HIV-1 provirus containing the packaging sequence and the position of the RRE upstream of the transgene cassette, but differed in other aspects, with later generations becoming U3/tat-independent, self-inactivating in the 3′LTR, and incorporating the use of the cppt and wPRE. The apparent lack of examples in engineering of the 5′packaging sequence is likely due to its complex structure and the condensed information encoded within that is necessary for many aspects of HIV-1 replication: transcription, balance in splicing, translation of GagPol, genome dimerization, assembly, reverse transcription, and integration. Within this complex region, the major splice donor (MSD) is embedded within the stem-loop 2 (SL2) region, between SL1 (dimerization loop) and SL3 (binding to Gag). The re-positioning of the RRE sequence (and associated splice acceptor 7 (sa7) within the envelope region) to immediately downstream of the packaging region in lentiviral vector genomes was thought to ‘offer’ the MSD a splice acceptor (sa7) in the absence of rev. It was assumed that during lentiviral vector production the supply of rev results in rev binding to the RRE and suppression of MSD splicing to sa7, and consequently production of unspliced, full length lentiviral vector genomic vRNA (FIG. 7A). However, the present inventors (FIG. 9Bii) and others (e.g. Cui et al. (1999), J. Virol., 73: 6171-6176) show that aberrant splicing from the MSD to splice acceptor sites within the transgenic sequences can be substantial, leading to relatively modest levels of unspliced vRNA (relative to total—see FIG. 7B) available for packaging into vector virions—in some cases less than 5%.

The inefficiencies in generating vRNA for packaging may not always be directly observed in lentiviral vector titres produced in transient transfection methods because of the delivery of the high numbers of vector genome plasmids to cells; titres of standard 3^{rd} generation vectors above 1×10⁷ TU/mL are routinely possible, even with this type of aberrant splicing occurring. However, the present inventors anticipate that for development of stable producer cell lines, where much lower numbers of integrated vector genome cassettes may be present, the issue of aberrant splicing will likely be more substantial in effect. Indeed, the present inventors typically find that the genome component is limiting in stable producer clones, and hypothesise that MSD activity may substantially contribute to this limitation.

There is a further perhaps less obvious consequence in generating aberrantly spliced mRNA resulting from the MSD into transgene cassettes: (increased) expression of the transgene cassette during production. Previously, the TRIP system was developed to repress transgene expression during lentiviral vector production (described in WO 2015/092440), which enables recoveries in vector titres that are linked in proportion to a negative effect of the specific transgene protein on vector production. The present inventors have found that efficient aberrant splicing (for example in standard lentiviral vectors containing EF1a driven cassettes—see FIG. 9) produces mRNAs that typically encode for the transgene. Without wishing to be bound by theory, for EF1a driven cassettes the MSD splices to the strong EF1a splice acceptor but for other promoter-UTR sequences the MSD ‘selects’ weaker cryptic splice acceptors, even in the presence of rev. The MSD appears to ‘look past’ the RRE-sa7 sequence in favour of other sites more central to the vector genome i.e. in the transgene promoter region. This may be a ‘residual’ property of the HIV-1 5′packaging region since in wild type HIV-1 the MSD typically splices to splice acceptors placed centrally and to the 3′end of the genome. It is possible that the MSD aberrantly splices at many places within the vector sequences downstream but that only mRNAs that pass nonsense-mediated decay rules (i.e. they appear to be legitimate mRNAs because they encode a protein [the transgene protein]) are transported to the cytoplasm (and/or are stable in the cytoplasm) where they are then translated. This creates additional burden on the TRIP system to maintain repression of transgene encoding mRNA, leading to less repressive control over a larger pool of mRNA. Moreover, it is likely that the use of tissue specific promoters (partly to avoid transgene expression during lentiviral vector production) will be ‘undone’ by the cytoplasmic appearance of translatable mRNA encoding the transgene by this mechanism of aberrant splicing. In essence, the transgene will be expressed by the (typically powerful) constitutive promoter that is driving the expression of the vector genome vRNA.

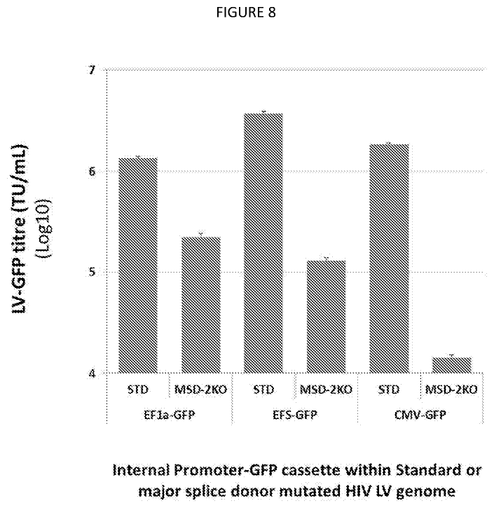

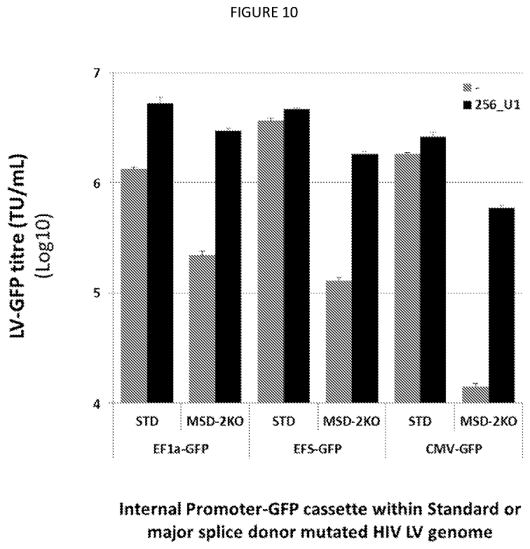

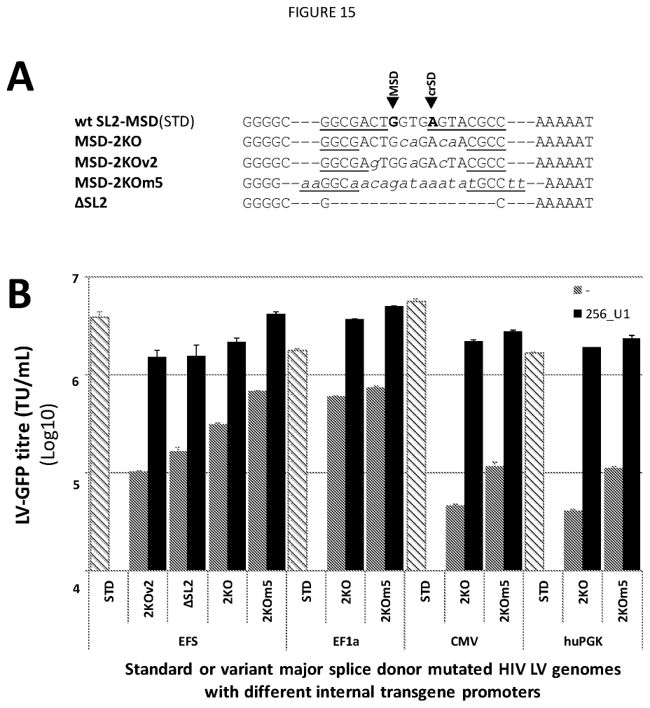

Therefore, there are many reasons to generate MSD-mutated lentiviral vectors, and indeed others have attempted to do so in U3/tat-independent lentiviral vectors without success. The present inventors have found that mutation of the MSD in HIV-1 activates an adjacent cryptic splice donor site within SL2, resulting in substantial levels of splicing. For this reason the present inventors have employed mutations in both the MSD and the nearby cryptic spice donor (crSD) (see FIG. 15), and refer to this modification as ‘MSD-2KO’ or ‘MSD2KO’ or ‘functional modification of the MSD’. It is shown in FIG. 9 that this double mutation is extremely effective in ablating aberrant splicing from the splicing region of SL2 (including both MSD and CrSD) to the strong EF1a splice acceptor during lentiviral vector production. It is also shown that the MSD-2KO lentiviral vector genome containing three different promoter-GFP transgene cassettes leads to reduced vector titres (FIG. 8), as similarly reported by others. In FIG. 9Bi, it is also demonstrated that provision of HIV-1 tat in trans is able to rescue the observed reduction in titre of the MSD-2KO lentiviral vector genome, although the amount of ‘aberrant’ splice product (from a minor cryptic splice donor in SL4) is increased (FIG. 9Bii). Importantly, it is shown that modified U1 snRNAs re-directed to different regions of the vector packaging signal increase MSD-2KO lentiviral vector genome titres, and without increasing the presence of the minor splice product (FIGS. 9 and 10).

### Example 7—Enhancement of MSD-2KO Lentiviral Vector Titres is not Due to Suppression of the 5′polyA Site within Vector Genome Cassettes

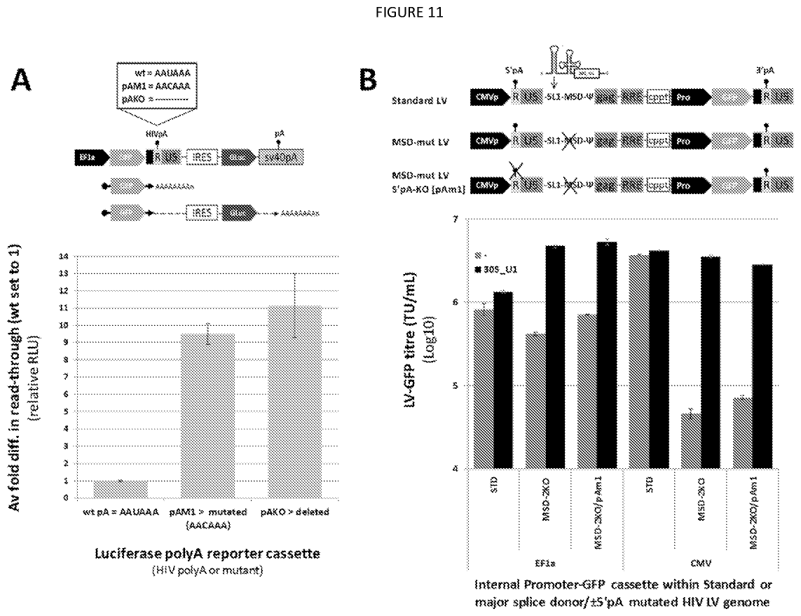

To assess if the present invention is acting to suppress the 5′polyA site, a functional mutation to the 5′polyA site was introduced within MSD-2KO lentiviral vector genomes, containing either EF1a or CMV driven GFP transgene cassettes (FIG. 11); the description of the pAm1 polyA mutation is in FIG. 2 (shown again in FIG. 11A for clarity), which demonstrates complete abolition polyadenylation activity. Surprisingly, the present inventors found that mutation of the 5′polyA signal only partially increased titres in the EF1a-GFP containing MSD-2KO lentiviral vector genome, and had virtually no effect in the CMV-GFP MSD2KO lentiviral vector genome, which seemed to be commensurate with the degree of attenuating effect of the MSD-2KO mutation (for EF1a-containing genomes, the MSD2KO mutation is less pronounced). Importantly, the supply of the 305U1 molecule in this experiment increased titres of both the standard lentiviral vector genomes (in which endogenous U1 snRNA is presumably capable of fully suppressing any residual 5′polyA activity) and the MSD2KO/pAm1 lentiviral vector genomes, which had no possible 5′polyA activity. This provides compelling evidence that the modified U1 snRNA, supplied to recover titres of MSD-mutated lentiviral vectors, is acting at a post-transcriptional step not previously described.

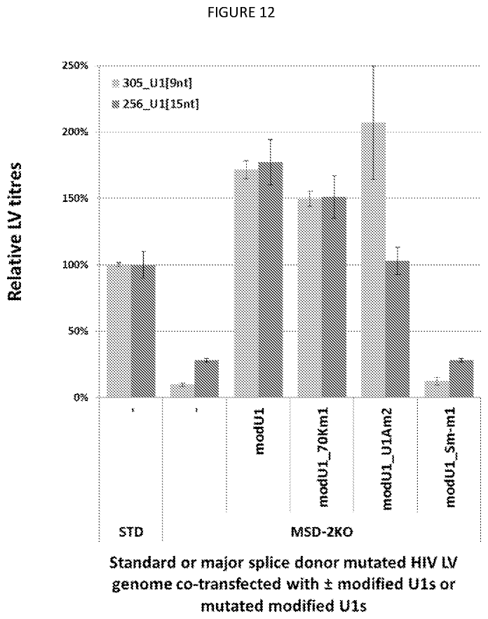

The present inventors then sought to mutate the 70K and U1A binding loops of the 305U1 and 256U1 in order to evaluate if this impacted the observed increase in titres of MSD2KO lentiviral vector genomes. FIG. 12 demonstrates that functional mutation of SL1 or SL2 within modified U1 snRNA has no effect on the ability of these molecules to augment MSD-2KO lentiviral vector titres when co-expressed during production; only the Sm protein binding mutation blocked this activity. This shows that the previously imperative 70K-binding properties of re-directed U1 snRNAs in suppressing polyA activity are not important in the invention, and provides further evidence that modified U1 snRNAs used to increase MSD-mutated lentiviral vector titres are functioning by a novel mechanism.

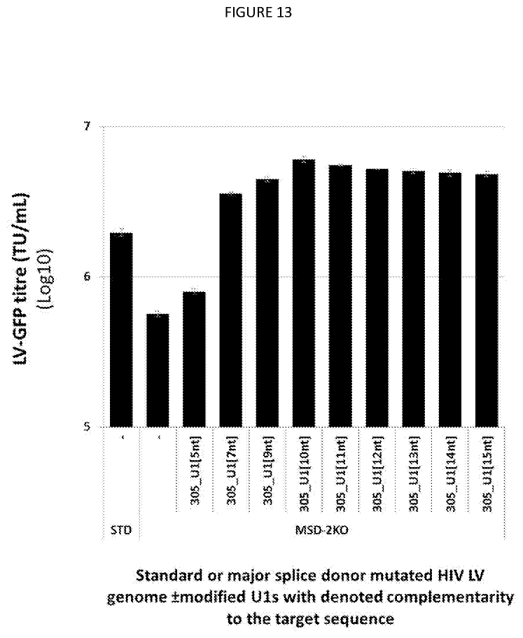

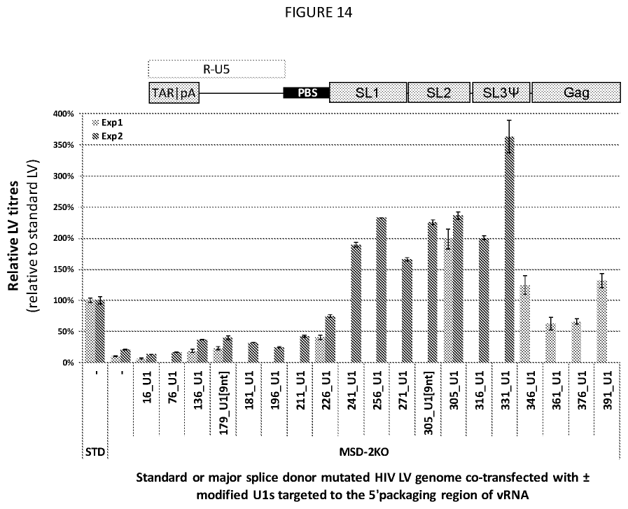

To assess if the titre increase mediated by the modified U1 snRNAs when applied to MSD-2KO lentiviral vectors differed in their ‘preferred’ target site compared to standard lentiviral vectors (see FIGS. 4&5), a panel of modified U1 snRNAs that targeted different sites (see Table I) along the 5′ region of the MSD-2KO lentiviral vector genome were screened (FIG. 14). This screen indicates that targeting to the packaging region is preferred (SL1-3), with perhaps a ‘hotspot’ within SL3. The screen was performed with modified U1 snRNA that had 15 nucleotides of complementarity to the target site (or 9 nucleotides were stated), as we had previously demonstrated for standard lentiviral vectors, the increase in titre may be more robust when using complementarity lengths of greater than 9 nucleotides (see FIG. 4). Indeed, we performed another experiment to show that for MSD-2KO lentiviral vectors, the titre boost observed with modified U1 snRNAs (targeting the ‘305’ sequence) could be observed with only 7 nucleotides of complementarity but that in preferred use it would be better to use 10-to-15 nucleotides of complementarity due to an increase in titre boost (FIG. 13), and also because this would minimize any possible ‘off-target’ effects by the modified U1 snRNAs.

### Example 8—Enhancement of MSD-2KO Lentiviral Vector Titres by Modified U1 snRNAs is not Dependent on the Type of Splice Donor Mutation

FIG. 15A displays the genetic modification to the SL2 loop of the ‘MSD2KO’ variant of MSD-mutated lentiviral vector genome packaging region, which mutates both the MSD and the cryptic splice donor positioned downstream (the MSD2KO variant has been utilized in many of the non-limiting examples herein). To assess if the effect in boosting titres by use of the modified U1 snRNAs was in anyway dependent on the specific changes made to the MSD2KO variant, we made three other splice donor region mutants: [1] ‘MSD2KOv2’, which also introduced two specific changes within the MSD and cryptic donor sequences, [2] ‘MSD-2KOm5’, which replaces the entire SL2 loop with an artificial stem loop; and [3] a complete SL2 deletion, thus removing the entire splice donor region (also termed the splicing region). We then produced standard or MSD-mutated lentiviral vector variants (containing an EFS-GFP expression cassette) in HEK293T cells+/−modified U1 snRNA (256U1), and titrated vector supernatants (FIG. 15B). The results show that all four MSD-mutated lentiviral vector variants were attenuated compared to the standard vector but that all four could be enhanced by use of modified U1 snRNA supplied during lentiviral vector production, indicating no specific sequence dependency of splice donor region mutation by the modified U1 snRNA. Interestingly, the MSD-2KOm5 variant was the least attenuated, and when produced in the presence of the 256U1 molecule gave the greatest increase in output titres, irrespective of the identity of the internal promoter employed (comparing EFS, EF1a, CMV and human PGK promoters).

### Example 9—the Use of a Modified U1 snRNA Cassette Encoded within a Lentiviral Vector Genome Plasmid DNA Backbone in Cis

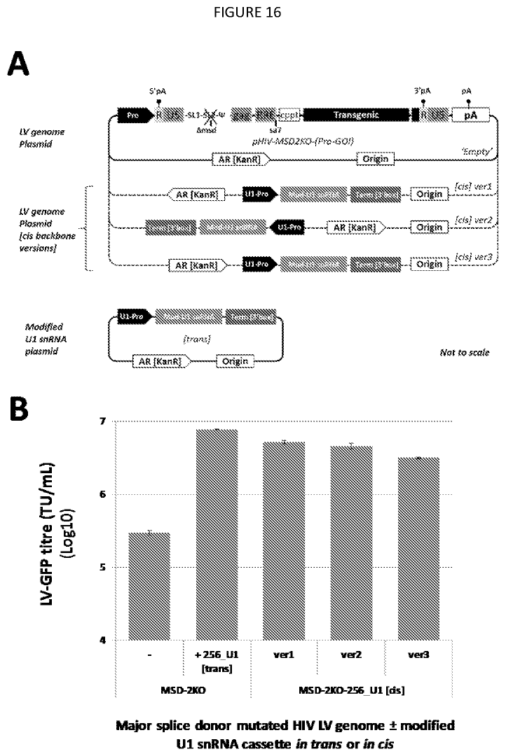

Previous examples herein have disclosed the use of modified U1 snRNA molecules during lentiviral vector production in trans by transient co-transfection of HEK293T cells with lentiviral vector component plasmids and modified U1 snRNA encoding plasmids. To evaluate if the MSD-mutated lentiviral vector genome cassette and modified U1 snRNA cassettes could be suitably encoded within the same plasmid DNA molecule, three variant constructs were cloned (FIG. 16A). An MSD-mutated lentiviral vector genome cassette (the MSD-2KO variant) was modified such that a 256U1 expression cassette was inserted in three different configurations relative to the lentiviral vector genome cassette and/or functional plasmid backbone sequences. These ‘cis’ version plasmids were used to make MSD-mutated lentiviral vectors in HEK293T cells and compared to the ‘trans’ mode, where the modified U1 snRNA plasmid was co-transfected with the unmodified MSD-mutated lentiviral vector genome (FIG. 16B). The results show that the titres of these ‘cis’ version plasmids was similar to the unmodified MSD-mutated lentiviral vector genome+256U1 supplied in co-transfection.

### Example 10—the Use of a Cell Line Stably Expressing a Modified U1 snRNA to Enhance Production of Both Standard or MSD-2KO Lentiviral Vectors

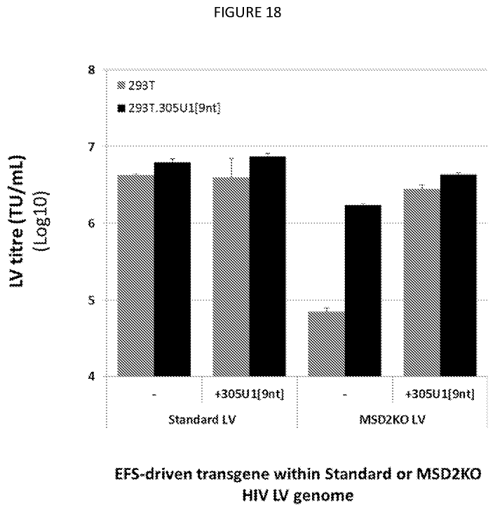

The 305U1 expression cassette was stably integrated into HEK293T cells, and standard or MSD-2KO lentiviral vectors produced by transient transfection+/−additional 305U1 plasmid DNA. The successful generation of stable cells reveal for the first time that modified U1 snRNA can be expressed endogenously within cells without cyctotoxic effect, indicating that modified U1 snRNAs do not titrate-out cellular factors involved in either U1 snRNA synthesis or the spliceosome, and either no off-targeting occurs or that off-targeting effects do not impact upon normal cell viability. The output titre of lentiviral vectors demonstrate that the titre increase mediated by modified U1 snRNAs on both standard and MSD-2KO lentiviral vectors is possible in stable provision of modified U1 snRNAs (FIG. 18). This will enable modified U1 snRNAs to be easily incorporated into lentiviral vector packaging and producer cell lines.

### Example 11—Further Examples of Use of Modified U1 snRNAs to Enhance Titres of Standard Lentiviral Vectors Encoding Therapeutic Transgene Cassettes

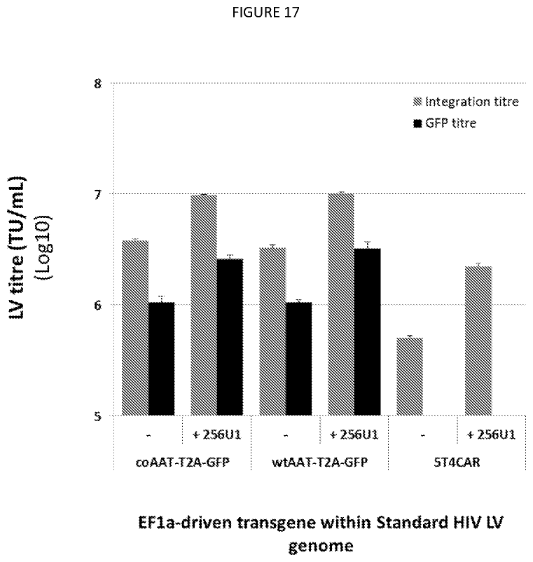

We produced standard lentiviral vectors encoding either wild type or codon-optimised human alpha1-antitrypsin (fused to a T2A-GFP reporter), or a chimeric antigen receptor (directed to 5T4), driven by an EF1a promoter cassette, in serum-free, suspension HEK293T cells+/−256U1. These vectors were titrated by integration assay or by flow cytometry to assess GFP expression in target cells (FIG. 17). These data show that 256U1 increased titres of all vectors tested.

### Example 12—MSD-Mutated Lentiviral Vectors Produce Less Transgene Protein During Production

A further advantage of ablating aberrant splicing during lentiviral vector production is to reduce the amount of transgene-encoding mRNA that leads to transgene protein production. Transgene expression can impact substantially on lentiviral vector production, which has led us to previously develop the TRiP system to suppress transgene translation during viral vector production (described in WO 2015/092440). In brief, the bacterial protein ‘TRAP’ is co-expressed during vector production and binds to its ‘TRAP binding sequence’ (tbs) inserted upstream of the transgene ORF within the 5′UTR—thus blocking the scanning ribosome.

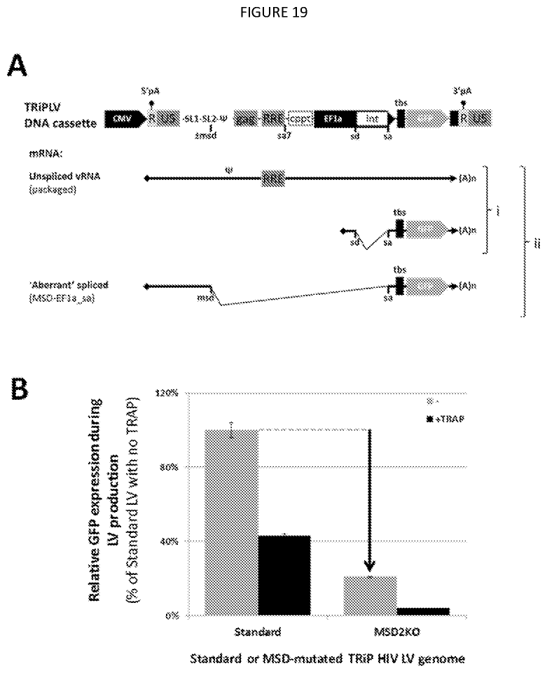

During the course of this work, we unexpectedly found that transgene-encoding mRNAs were effectively produced from the ‘external’ (CMV) promoter driving the vector genome cassette due to splicing-out from the major donor splice region of the SL2 to internal splice acceptor sites. The degree to which this occurs depends on the internal sequences between the cppt and the transgene ORF (i.e. the promoter-5′UTR sequence). The use of the EF1a promoter (containing a very strong splice acceptor) in the transgene cassette, results in aberrant splicing from the MSD in over 95% of total transcripts originating from the external promoter (see FIG. 7). By comparing total GFP expression in standard or MSD-2KO lentiviral vector production cultures (FIG. 19), we show that up to 80% of the transgene protein expressed during production originates from the aberrant splice product. We found that combining the MSD-2KO genotype with the TRiP system augmented the reduction in transgene protein produced.

### Example 13

Co-expression of modified U1 snRNAs targeted to the vRNA of lentiviral vectors results in increase vRNA within vector particle samples.

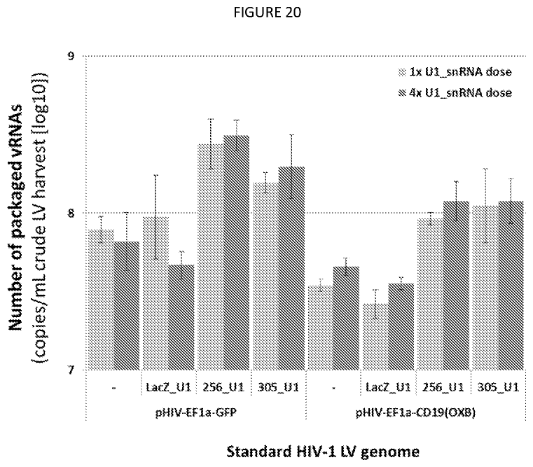

Experiment performed in adherent HEK293T vector production setting. Standard lentiviral vectors encoding GFP (pHIV-EF1a-GFP) or a chimeric antigen receptor to CD19 (pHIV-EF1a-CD19) were produced in the absence or the presence of modified U1 snRNAs targeting either sites within the lentiviral vector packaging region (256U1 or 305U1) or a LacZ control (LacZU1). Modified U1 snRNAs are named according to the first nucleotide of the targeting sequence site within the lentiviral vector packaging region (see Table I). The modified U1 snRNA expression constructs were supplied at two different doses: 1× and 4×. The vector supernatants were treated with DNAase/RNAse prior to extraction of total RNA from vector particles and then RT-qPCR against the packaging region (Psi) of the vRNA in order to quantify total vRNA copies present (FIG. 20). The data indicates that the co-expression of modified U1 sRNAs resulted in an increase in vRNA within vector particles.

### Example 14

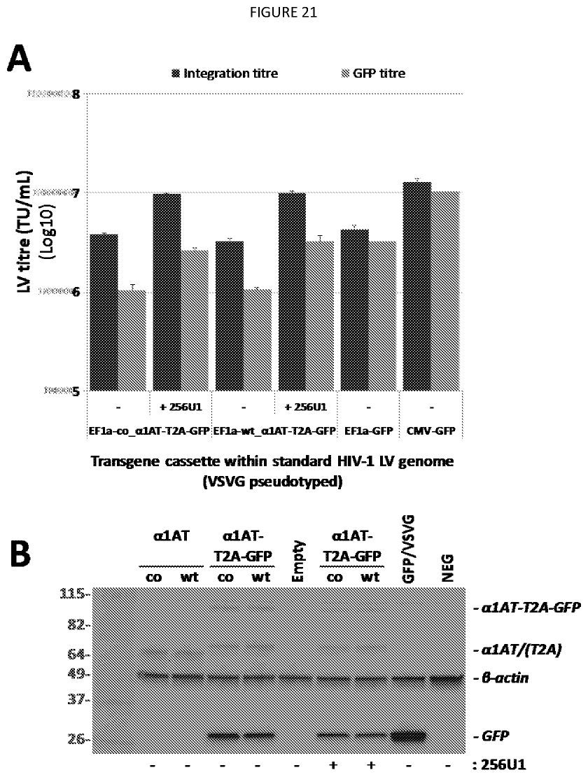

Co-expression of modified U1 snRNA targeted to the vRNA of lentiviral vectors can lead to reduced transgene expression during vector production. Experiment performed in suspension, serum-free adapted HEK293T vector production setting. During evaluation of modified U1 snRNA-enhanced-production of HIV-1 based lentiviral vectors encoding the α1-anti-trypsin (a1AT) transgene (fused to GFP) (FIGS. 21 & 22), it was observed that the relative increase in LV titres was greater for vector pseudotyped with Sendai envelope (F/HN) (˜20-fold) than for vector pseudotyped with VSVG (˜3-fold). Vector was produced in suspension HEK293T cells (and adherent HEK293T cells where indicated), and then titrated on adherent HEK293T cells by both integration assay and by flow cytometry. Post-production cell lysates were immunoblotted for the transgene protein (and GFP) as well as β-actin, which revealed a modest, yet consistent suppression of transgene expression when 256U1 was provided in trans. Since Sendai F protein requires trypsin-activation prior to transduction, this result indicated that the presence of α1AT in the harvest material inhibited trypsin-activation of the F protein. Therefore, the apparent suppression of α1AT expression in cells transfected with the p256U1 provided an additional boost to active vector titres. Without wishing to be bound by theory, this result is consistent with a mechanism of action by 256U1 whereby vRNA is not only stabilised (perhaps avoiding nuclear degradation) but may be diverted away from translation, which would otherwise lead to production of transgene protein. Indeed, it has recently been reported that the pool of non-translating, full length unspliced wild type HIV-1 is actively packaged into virions (Chen et al. (2020), PNAS; 117(11): 6145-6155). In this specific case, these two surprising effects of 256U1 are additive, leading to a 10-fold increase in vector output/activity. For other therapeutic vector genomes, where transgene expression may be detrimental to output vector titres, this modest effect of reducing transgene expression by modified U1 snRNA may have a similar effect of contributing to titre increase.

### Example 15

Use of modified U1 snRNAs to increase titres of lentiviral vectors containing an inverted transgene cassette.

In some cases, it is necessary to utilise lentiviral vector genomes wherein the transgene cassette is inverted i.e. the transcription unit is opposed to the promoter driving the vector genome cassette. For example, most lentiviral vectors developed for treatment of Sickle cell disease or β-thalassemia comprise a β-globin gene, wherein the introns are encoded in context with the three exons (this is because out-splicing of the introns in terminally differentiated erythrocytes is required for efficient β-globin expression). In most cases, introns can be retained within packaged lentiviral vector vRNA containing the rev response element (RRE) as binding of rev to the RRE in the nucleus results in export of intron-containing vRNA; however, this is not the case for the β-globin gene, and the introns are lost from these vRNAs when the transgene cassette is in the ‘forward’ orientation, even in the presence of rev/RRE. In addition, there may be instances where one component of the transgene cassette may be in reverse and another in the forward orientation, for example use of bi-directional transgene cassettes, or multiple separate cassettes.

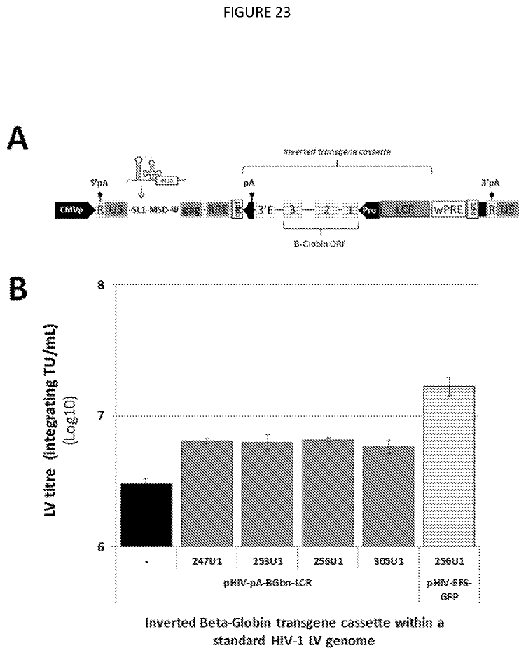

To assess if the use of modified U1 snRNA enables increase of lentiviral vectors comprising an inverted transgene cassette, a β-globin gene containing lentiviral vector was produced in suspension (serum-free) HEK293T cells in the absence or presence of four different modified U1 snRNAs targeted to the packaging region (FIG. 23). Experiment performed in suspension, serum-free adapted HEK293T vector production setting. The data demonstrates that modified U1 snRNAs are also able to enhance titres of these class of lentiviral vectors.

### Example 16

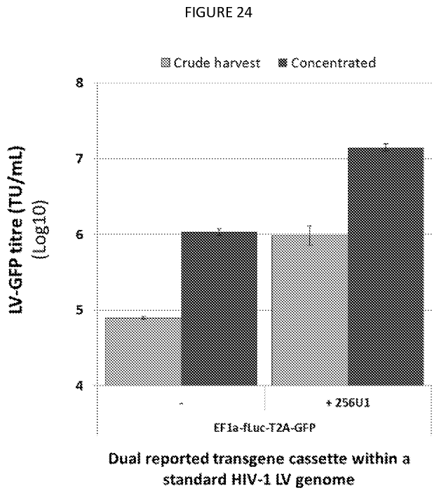

The enhancement of lentiviral vector titre by modified U1 snRNA is measurable in concentrated vector preparations. Experiment performed in suspension, serum-free adapted HEK293T vector production setting. Lentiviral vector encoding a firefly luciferase-GFP double reporter transgene cassette was produced in suspension (serum-free) HEK293T cells in the absence or presence of 256U1. Clarified vector harvest was concentrated by centrifugation (˜20-fold concentration factor), and then both pre- and post-concentrated vector samples titrated by transduction of adherent HEK293T cells, followed by flow cytometry (FIG. 24). The data show that the increase in titre imposed by modified U1 snRNA is observed in process LV material, further adding evidence that the boost in vector titre is not an artefact associated with crude vector material.

### Example 17

Development of a highly sensitive, quantitative method of detection of modified U1 snRNA towards measurement of residual modified U1 snRNA in post-production cell lysates and vector material. After pre-snRNA is processed in the cytoplasm it is transported back into the nucleus as part of the spliceosome, which is the key complex involved in pre-mRNA splicing. The modified U1 snRNAs as described herein are therefore expected to be mainly nuclear in location, and their potential for incorporation into virions is expected to be very low. Indeed, others have shown that processed U1 snRNA is not actively packaged into HIV-1 virions, and the presence of pre-U1 snRNA is 100-fold lower than the most abundant cellular RNAs detected in virions, such as 7SL (Eckwahl et al (2016); RNA, 22(8): 1228-1238).

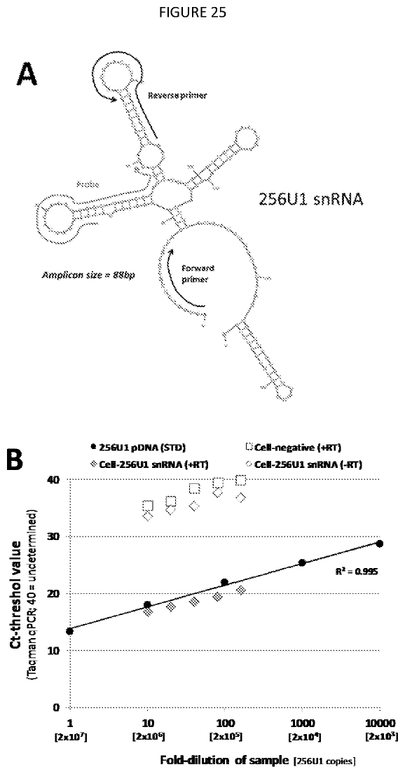

Nevertheless, to be able to assess the expression of modified U1 snRNAs within cells and detect/quantify residual RNA derived from modified U1 snRNAs in vector product, a Taqman-based RT-qPCR assay was developed (FIG. 25). In order to differentiate modified U1 snRNA from the highly expressed endogenous U1 snRNA, an amplicon was designed such that the forward primer is homologous to the vRNA targeting sequence at the 5′ terminus of the modified U1 snRNA molecule (see FIG. 25A). Therefore, whilst the reverse primer will enable cDNA synthesis of both endogenous and modified U1 snRNA, the quantitative PCR step will only occur from cDNA derived from the modified U1 snRNA. In this non-limiting example, an 88 bp amplicon was designed to amplify 256U1 snRNA. To assess the sensitivity of the primer/probe set suspension (serum-free) HEK293T cells were transfected with p256U1 and total RNA extracted and purified from replicate cultures treated with Benzonase (to degrade residual p256U1 DNA). Taqman qPCR was performed on purified total RNA+/−the reverse transcriptase step in order to assess signal being derived from undigested p256U1 DNA (see FIG. 25B). The p256U1 plasmid was used as a standard curve for the qPCR step, and displayed very good linearity and range. Diluted cDNA samples from untransfected cell RNA and p256U1-transfected cell RNA (+RT treated) generated Ct values of 19-20 cycles difference, with the −RT treated p256U1-transfected cell RNA only ˜2-fold lower Ct than untransfected cell RNA. This demonstrated that the RT-qPCR assay was able to unambiguously detect and quantify modified U1 snRNA over endogenous U1 snRNA.

### Example 18

The modified U1 snRNA dose response and use of multi-variance modelling to optimise modified U1 snRNA plasmid and LV component plasmid ratios for maximal transient transfection of suspension (serum-free) HEK293T cells.

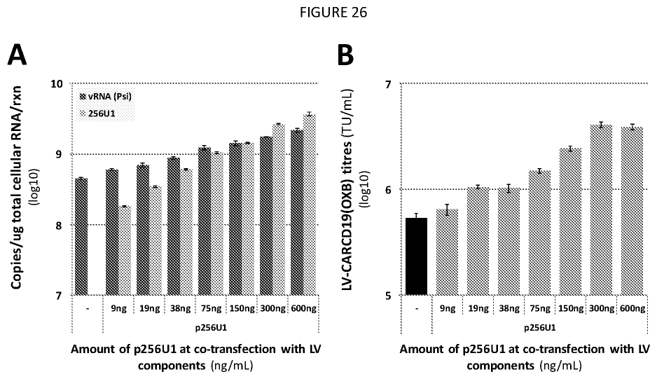

To assess the relationship between input modified U1 snRNA plasmid during transient transfection, resultant modified U1 snRNA expression and the impact on both vector genome RNA (vRNA) and output vector titres, a simple dose response study was performed. An LV encoding an EF1a-promoter driven CAR-CD19 expression cassette was produced in suspension (serum-free) HEK293T cells by transient transfection of LV component pDNA, with the input of p256U1 set over a range of amounts from zero to 600 ng/mL (effective final culture concentration at transfection). All LV pDNA ratio/amounts remained constant, whilst total pDNA was maintained by filling with pBlueScript. Post-production cells were analysed by extracting total RNA and quantifying both 256U1 snRNA and vRNA, as well as an endogenous transcript (RPH1; data not shown) to normalise for total RNA within the cDNA step (FIG. 26A). Additionally, clarified LV supernatants were titrated by integration assay (FIG. 26B). The data indicate a linear correlation between input p256U1 amount, the expression levels of 256U1 snRNA and the steady state levels of vRNA (which were increased in a linear fashion). The output titres of LV-CARCD19 also displayed a linear relationship with input p256U1 amounts (and vRNA increase within the production cell), up to an input level of 300 ng/mL where titre increase became maximal. This suggested that the minimum input level of p256U1 that achieve maximum titre increase (under these conditions and for this LV genome) was somewhere between 150 and 300 ng/mL.

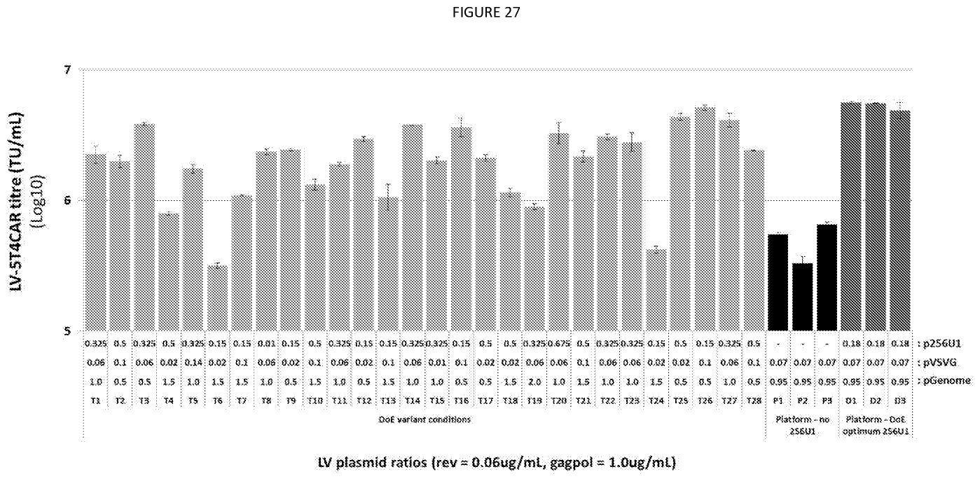

Design of Experiment (DoE) was used to generate and test 28 conditions at 40 mL shake flask scale, wherein p256U1, pGenome (in this case pHIV-EF1a-5T4CAR) and pVSVG were varied, and pGagPol/pRev input levels were kept constant (FIG. 27). Clarified vector harvests were titrated by transduction of adherent HEK293T cells, followed by immuno-flow cytometry using a MAb against 5T4CAR. The optimum input of 180 ng/mL p256U1 identified by DoE was then applied to generation of further HIV-EF1a-5T4CAR vector preps in comparison to no p256U1, resulting in an average increase in LV output titres of ˜10-fold. The 180 ng/mL input amount of p256U1 was in close agreement with the findings of the dose response experiment (see FIG. 26).

### Example 19

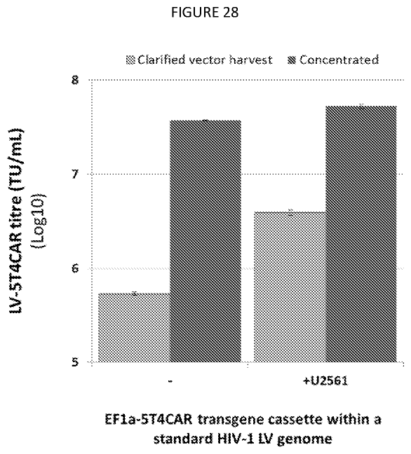

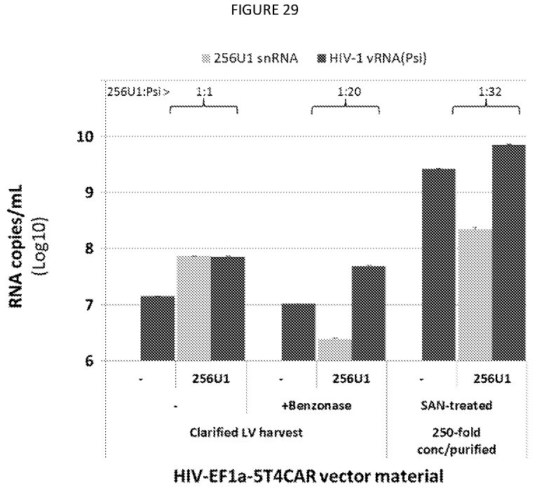

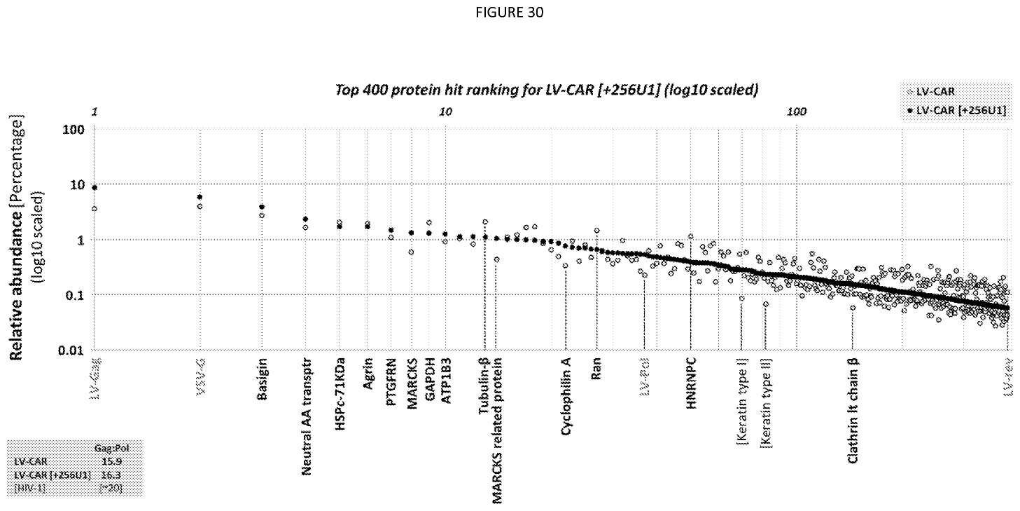

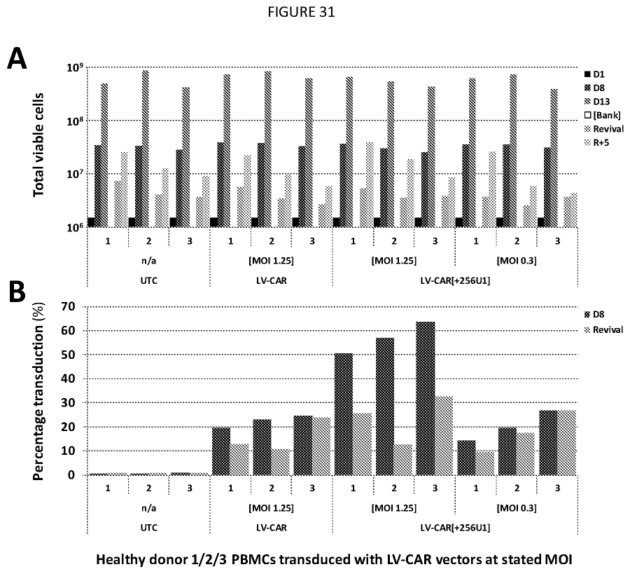

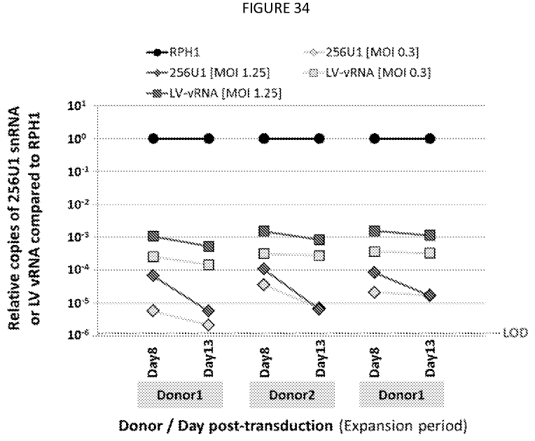

A case study: generation and assessment of CAR-T cells using concentrated/purified lentiviral vector preparations produced with or without 256U1. To assess the impact of the application of modified U1 snRNA on a lentiviral vector gene therapy product, a small case study was performed whereby a lentiviral vector encoding a chimeric antigen receptor targeted to 5T4 (HIV-EF1a-5TACAR/′LV-CAR′) was [1] produced with or without 256U1, and purified (FIG. 28), [2] the amount of residual 256U1 snRNA in concentrated product quantified (FIG. 29), [3] a comparative protein analysis of the two concentrated vector preparations performed (FIG. 30 & Table IV), [4] primary T-cells expanded from three healthy donor PBMCs were transduced and assessed for killing activity against 5T4-positive cells (FIGS. 31-33), and [5] residual 256U1 and vRNA in CAR-T expansion cultures assessed by RT-qPCR (FIG. 34).

Two concentrated HIV-EF1a-5TACAR vector preparations were generated in suspension (serum-free) HEK293T cell cultures at 250 mL shake flask scale with either p256U1 plasmid DNA added (+256U1) or with pBluescript as a negative control. Clarified harvest (non-treated with Benzonase) was then subjected to ion-exchange chromatography resulting in concentration by a factor of ˜125-fold, and then subjected to nuclease treatment. Finally, the vector preparations were subjected to centrifugation for ˜45 minutes, before resuspension in TSSM resulting in a 250-fold total concentration. Both the clarified harvest and concentrated vector samples were titrated by transduction of adherent HEK293T cells followed by immuno-flow cytometry using an antibody against the CAR transgene (FIG. 28). These data confirm the titre increase mediated by use of 256U1 snRNA.

Vector samples from the concentration process were then subjected to total RNA extraction, with clarified harvest samples being treated+/−Benzonase prior to extraction. Then both vRNA and residual 256U1 snRNA were quantified by RT-qPCR (see Example 17 for development of 256U1 RT-qPCR assay). FIG. 29 displays this data, which shows [1] the increase in vRNA within all vector samples from vector made in the presence of 256U1, [2] the impact of nuclease treatment on the abundance of vRNA compared to 256U1 snRNA, and [3] the relative ratio of 256U1 snRNA signal compared to vRNA within concentrated vector. This analysis reveals that the 256U1 snRNA signal present in clarified LV harvest is primarily ‘free’ RNA that has presumably originated from leaky/burst cells during production because benzonase treatment dramatically reduces this detection. In concentrated LV material the ratio of 256U1-to-vRNA signal is 1-to-32, indicating that for every sixteen vRNA-containing LV particles a single 256U1 snRNA (or at least the 88 bp amplicon) is present. This suggested that 256U1 snRNA is not actively packaged into LV particles, and that the signal detected within concentrated LV material is likely due to the presence of residual 256U1 snRNA on the outside of particles. This also suggests that further reduction in 256U1 snRNA signal may be possible with optimisation of nuclease treatment and LV polishing steps.

The two concentrated LV-CAR vector preparations were then analysed by mass spectrometry in order to assess any major impact of the expression of the 256U1 snRNA during LV-CAR production on the protein mark-up of the virions. Samples were prepared as described herein (see section ‘General molecular/cell biology techniques and assays’) and peptides analysed on a on an UltiMate 3000 coupled online to a Q Exactive™ HF mass spectrometer. The mass spectra were analyzed in DIA-NN (Version 1.7). Protein sequences of Homo sapiens uniprot reference proteome were concatenated with lentiviral proteins (gag, pol, rev) and the VSV-G glycoprotein, and frequent contaminants to generate a predicted spectral library for this project. The top 400 proteins detected in the LV-CAR[+256U1] vector sample were ranked and their relative abundance plotted as a percentage of total spectral abundance of the top 400 proteins (FIG. 17, solid black circles). The relative abundances of the same protein hits within the LV-CAR vector sample were also plotted to assess if any major differences in protein profile of the two vector preparations could be visualised (FIG. 17, open circles). These data confirm the presence of expected lentiviral proteins gag (rank #1), VSV-G (rank #2) and pol (rank #37), with rev appearing at rank #400. A comparison of the ratio of gag to pol peptides revealed a very similar ratio of ˜16:1 for both samples, which is close to the accepted rate of differential translation of the gag/pol mRNA (in wild type HIV-1) of 20:1 (gag polyprotein is the primary product of translation but approximately 1-in-20 times, a frameshift event results in translation of the gagpol polyprotein). Also present were cellular proteins known to be incorporated into HIV-1 based lentiviral virions: Basigin (rank #3), HSPc-71K(rank #5) and Cyclophilin A (rank #22; binds specifically to capsid). The analysis revealed minimal difference between the two LV-CAR vectors, indicating that the over-expression of the 256U1 snRNA during LV production did not lead to any gross up/down-regulation and/or incorporation of cellular proteins into LV virions.

Differential expression analysis for hits that differed significantly between the two LV samples by up to 2-fold was conducted in R using the BioConductor DEP package. A selection of the major hits from this analysis is displayed in Table IV. This statistical analysis revealed that the 2-fold increase of gag, pol, VSVG and Cyclophilin A was highly significant, suggesting that these LV proteins were more abundant in the LV-CAR[+256U1] vector relative to other background (possible contaminating) proteins.

TABLE IV
A selection of proteins from statistical analysis performed on
hits varying by up to 2-fold from the comparative protein analysis
of concentrated/purified preparations of HIV-EF1a-5T4CAR (LV-
CAR) produced +/−256U1 by mass spectrometry. Differential
expression analysis was conducted in R using the BioConductor
DEP package. Proteins with missing values were initially filtered
such that at least one condition had quantification for all replicates.
Variance stabilization normalization was performed using the
BioConductor VSN package. Missing values were then imputed using
Quantile Regression Imputation of Left-Censored data (QRILC).
Differential expression analysis was performed using protein-
wise linear models with empirical Bayes statistics. P-values were
adjusted for multiple testing using the Benjamini & Hochberg
method. The ranking numbers are the top 400 proteins detected
in the LV-CAR[+256U1] sample.

| | Fold difference: | | |
|---|---|---|---|
| | LV-CAR[+256U1] | Adj p | |
| | vs LV-CAR | value | Rank |
| LV-Gag | 1.97 | 4.70E−09 | 1 |
| VSVG | 1.21 | 9.18E−06 | 2 |
| Basigin | 1.23 | 1.39E−03 | 3 |
| Neutral AA transporter B(0) | 1.14 | 3.26E−03 | 4 |
| MARCKS | 1.44 | 3.48E−02 | 8 |
| MARCKS-related protein | 1.42 | 2.00E−02 | 12 |
| Cyclophilin A | 1.84 | 2.32E−09 | 22 |
| LV-Pol | 1.92 | 2.33E−13 | 37 |
| Clathrin light chain β | 1.85 | 3.82E−10 | 145 |
| 60S ribosomal protein L18 | −2.11 | 7.20E−05 | 286 |
| 60S ribosomal protein L15 | −1.11 | 2.06E−03 | 382 |

The two concentrated HIV-EF1a-5TACAR/LV-CAR vector preparations were used to transduce Peripheral blood mononuclear cells (PBMCs) from three healthy donors at an MOI of 1.25 (both vector preps) or MOI 0.3 (just HIV-EF1a-5TACAR+256U1), followed by expansion of transduced T-cells and cell banking at day 13. Cells were then revived and expanded for a further 5-6 days. Total viable cells and percentage transduction by the LV-CAR or LV-CAR[+256U1] vectors during expansion and post-revival were monitored (FIG. 31). This analysis showed that despite using matched MOIs of 1.25 for both LV preparations, the LV-CAR[+256U1] vector generally transduced the expanded T-cells more efficiently than the LV-CAR vector (this perhaps was due to less contaminants in the LV-CAR[+256U1] vector—see FIG. 30 & Table IV). The percentage transduction with the LV-CAR[+256U1] vector at MOI 0.3 was similar to those transduced with the LV-CAR vector (MOI 1.25), and therefore further comparisons were focused on these samples.

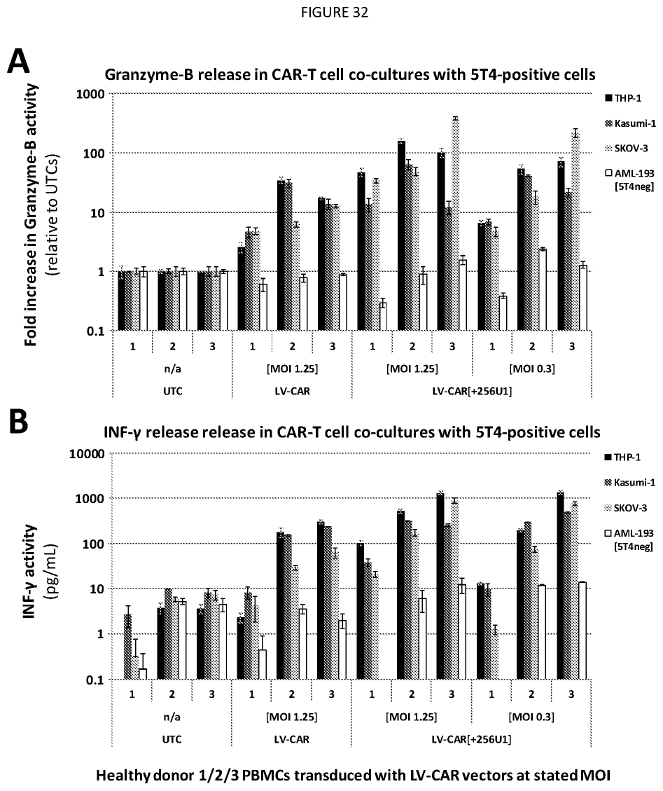

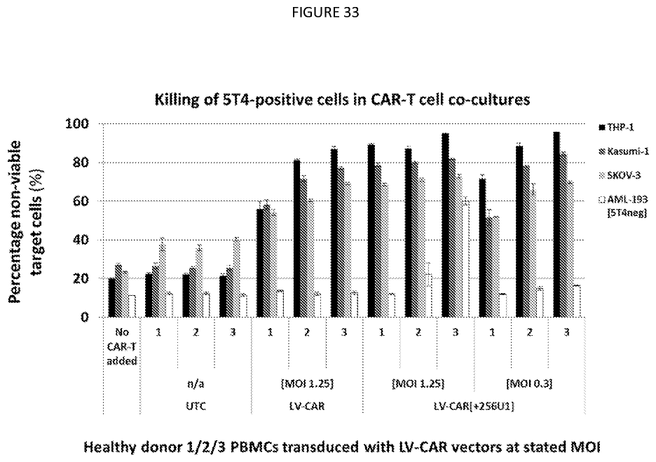

The 5T4CAR-expressing T-cells were then evaluated for target cell killing activity by co-incubation with equal numbers of 5T4-positive cells (THP-1, Kasumi-1, SKOV-3) or 5T4-negative cells (AML-193), followed by analysis of cytokine release at 24 hours post-incubation (FIG. 32) and cell killing at 40 hours post-incubation (FIG. 33). These results show that CAR-T cells transduced with either of the two vector preparations were equally capable of being activated specifically in the presence of 5T4-positive cells by releasing Granzyme-B and Interferon-v, resulting in specific cell killing.

At Day 8 and Day 13 post-transduction, cell samples were taken and total RNA extracted in order to assess the relative abundance of any residual 256U1 snRNA compared to RPH1 mRNA used as a cellular transcript loading control. FIG. 34 displays these data, revealing that relative to the RPH1 transcript at each time point, the residual 256U1 snRNA signal was 4-5 logs lower, and residual vRNA was 3-3.5 logs lower. The reduction in residual 256U1 snRNA signal between day 8 and day 13 was ˜5 fold greater than for vRNA, indicating that a large proportion of the residual 256U1 snRNA was exposed (for degradation) compared to vRNA (apparently protected within capsid), which probably reflects the presence of low numbers of intact LV virions remaining associated with T-cells during expansion. Altogether, the residual RNA analysis reveals that whilst modified U1 snRNA can be detected within processed LV material at low levels (1 for every 16 full virion in this research-level production scale), at least 80% of this signal is likely to be present externally of virions. This implies that further reduction of this residual is likely to be possible with optimisation of nuclease treatment and purification steps.

Given that constitutive, long term modified U1 snRNA expression within HEK293T cell lines is possible without apparent general cytotoxicity (see Example 21), it's likely that the delivery of full length modified U1 snRNA to a small fraction of target cells during LV transduction will have no impact on target cell viability. Due to the design of the modified U1 snRNA, no specific interaction with host cell RNA is thought possible, and its relative abundance in the target cell compared to the enormous pool of endogenous U1 snRNA would mean it highly unlikely to be able to efficiently compete for association with RNP factors.

### Example 20

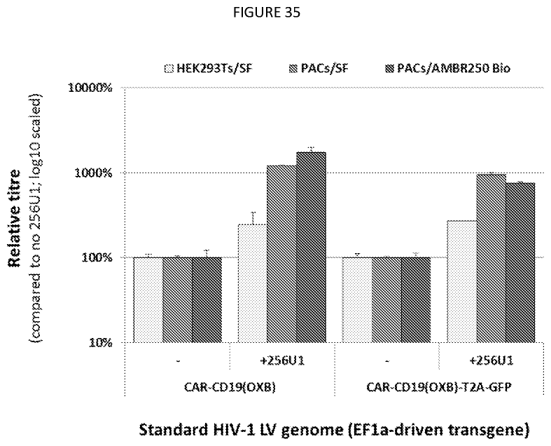

Use of modified U1 snRNA to increase LV production from suspension (serum-free) packaging cells. HIV-EF1a-CAR-CD19 vector or a GFP-reporter variant vector (HIV-EF1a-CAR-CD19-T2A-GFP) was produced in a lentiviral vector packaging cell line (PAC) either in shake flasks or 250 mL bioreactors (AMBR250), in the presence or absence of p256U1. HEK293T cells were transfected with all vector components in parallel as a control. Clarified vector harvests were titrated by transduction of adherent HEK293 Ts, followed by integration assay; titres were plotted relative to ‘no 256U1’ (FIG. 35). The data show that the boost in vector titre was greatest for the PAC cell line compared to the HEK293T cells.

### Example 21

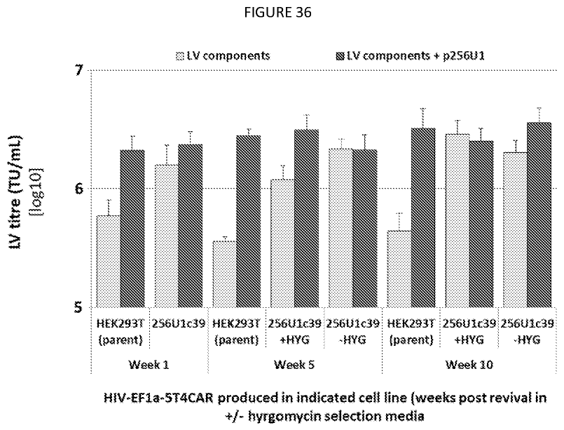

Lentiviral vector titre enhancement by suspension (serum-free) HEK293T cells stably transfected with a 256U1 expression cassette. Suspension (serum-free) HEK293T cells were stably transfected with a 256U1-HygR expression cassette and clones selected; clone 256U1c39 was chosen for evaluation. Lentiviral vector titre enhancement was assessed at week 1, 5 and 10 weeks post-isolation and continued growth in +/−hyromycin-B, and compared with the parental HEK293T cells at each time point by producing HIV-EF1a-5T4CAR vector by transient transfection, +/−p256U1 (FIG. 36). The data indicate that 256U1 snRNA expression within the 256U1c39 clone was stable with or without hygromycin selection, indicating that long term expression of these modified U1 snRNAs is not toxic to HEK293T cells. In addition, the level of vector titre enhancement in the 256U1c39 cell line was close to maximal observed titre boost across all conditions.

### Example 22

The enhancement effect of modified U1 snRNAs on lentiviral vectors does not appear to be dependent on the conserved 5′ dinucleotide ‘AU’ present in endogenous U1 snRNA.

In Example 1 it is shown that the mechanism of action by modified U1 snRNAs is not due to polyA suppression (a known property of endogenous U1 snRNA) of the 5′ polyA signal within the 5′LTR region of lentiviral vectors, because modified U1 snRNAs are still able to increase the titres of LVs containing functional mutation of this polyA site.

Others have characterised one aspect of U1 snRNA biology that appears to be important for its role in splicing (Yeh et al (2017) Nucleic Acids Res. 45(16): 9679-9693). Endogenous U1 snRNA recruits the CAP-binding complex (CBC) to its 5′ terminus, which is important for the functionality of the U1 snRNP in splicing; the authors found that the ‘AU’ dinucleotide provides optimal binding of the CBC when compared to other dinucleotides, and made a strong case for why the ‘AU’ dinucleotide is so widely conserved within eukaryotes.

To assess the importance of the conserved ‘AU’ dinucleotide sequence in the context of modified U1 snRNAs targeted to the packaging signal of a lentiviral vector genome, a number of variants were generated based upon the 256U1 snRNA (Table V).

TABLE V

| Variant ID | Sequence | GC % | Tm° C. |
|---|---|---|---|
| [Target in LV genome] | 3-gagAACGGCACGCGCGAAgtc-5 | 66.7 | 47.4 |
| | (SEQ ID NO: 75) | | |
| Base pairing | \|\|\|\|\|\|\|\|\|\|\|\|\|\|\| | | |
| 256_15_at | 5-atTTGCCGTGCGCGCTT -3 | 76.9 | 46 |
| | (SEQ ID NO: 76) | | |
| 256_13_aT | 5- aTGCCGTGCGCGCT -3 | 76.9 | 46 |
| | (SEQ ID NO: 77) | | |
| 256_13_ag | 5- agGCCGTGCGCGCTT -3 | 76.9 | 46 |
| | (SEQ ID NO: 78) | | |
| 256_13_*aa | 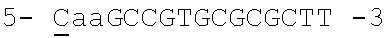 | 76.9 | 46 |
| | (SEQ ID NO: 79) | | |
| 256_13_ac | 5- acGCCGTGCGCGCTT -3 | 76.9 | 46 |
| | (SEQ ID NO: 80) | | |
| 256_13_gg | 5- ggGCCGTGCGCGCTT -3 | 76.9 | 46 |
| | (SEQ ID NO: 81) | | |
| 256_13_ga | 5- gaGCCGTGCGCGCTT -3 | 76.9 | 46 |
| | (SEQ ID NO: 82) | | |
| 256_13_gT | 5- gTGCCGTGCGCGCT -3 | 76.9 | 46 |
| | (SEQ ID NO: 83) | | |
| 256_13_gc | 5- gcGCCGTGCGCGCTT -3 | 76.9 | 46 |
| | (SEQ ID NO: 84) | | |
| 256_13_*Tc | 5- TgGCCGTGCGCGCTT -3 | 76.9 | 46 |
| | (SEQ ID NO: 85) | | |
| 256_13_*cg | 5- cgGCCGTGCGCGCTT -3 | 76.9 | 46 |
| | (SEQ ID NO: 86) | | |
| 256_13_*cc | 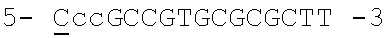 | 76.9 | 46 |
| | (SEQ ID NO: 87) | | |
| 256_13_GC | 5- GCCGTGCGCGCTT -3 | 76.9 | 46 |
| | (SEQ ID NO: 88) | | |
| 256_13_Ga | 5- GaCGTGCGCGCTTC-3 | 71.4 | 46.1 |
| | (SEQ ID NO: 89) | | |
| 256_13_Gt | 5- GtCGTGCGCGCTTC-3 | 71.4 | 46.1 |
| | (SEQ ID NO: 90) | | |
| 256_13_Gg | 5- GgCGTGCGCGCTTC-3 | 78.6 | 46.1 |
| | (SEQ ID NO: 91) | | |
| [U1 Pro TSS] | 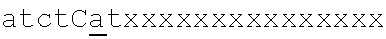 | | |
| | (SEQ ID NO: 92) | | |

Variant modified U1 snRNAs targeting position 256 of the HIV-1 LV vRNA genome generated to assess the impact of 5′terminus dinucleotide changes on vector titre increases. The table indicates the 15-nucleotide variant 256U1 target sequence and base pairing in the vRNA, and how the 13-nucleotide variant targeting sequences compare. The 256U1_13
variants were designed to maintain 13 contiguous base pairs with the target where possible so that calculated T-melting temperature (Tm° C.) was ~46° C. The dinucleotides at the 5′end of the modified U1 snRNA molecule are denoted, with underlined nucleotides representing likely transcription start site based on the findings of Yeh et al (2017); variants marked with an asterix indicate that the first of the two state dinucleotides are likely not the first nucleotide of the modified snRNA. The transcription start site of the U1 promoter is also indicated, showing the likely −1) TSS (grey-boxed ‘C’) for variants with ‘aa’ and ‘cc’.

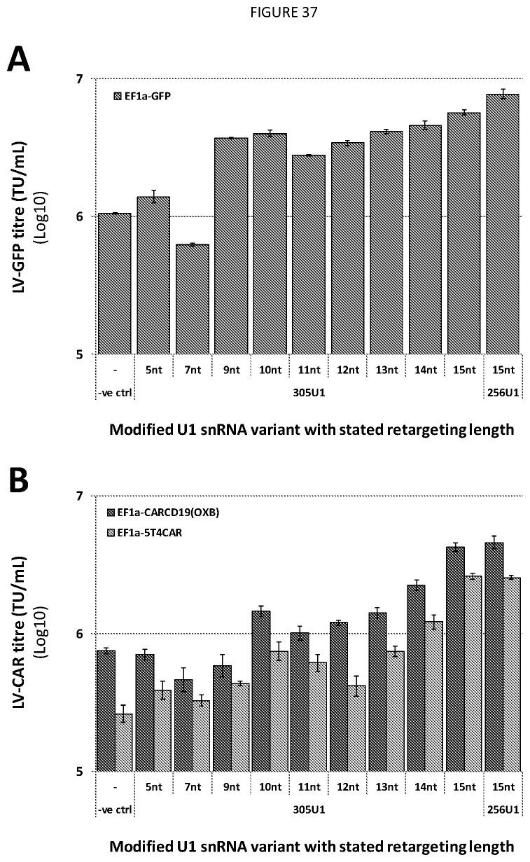

Yeh and colleagues (2017) also reported on the impact of transcription start site and U1 snRNA abundance when altering the first 1-2 nucleotides. They found that transcription initiation was favoured at purines over pyrimidines, and so when pyrimidine-purine or pyrimidine-pyrimidine dinucleotides were placed at positions 1 and 2, the pyrimidines were ‘skipped’ in favour of the next purine. Exceptions to this general rule were ‘UU’, for which transcription initiation occurred 19 or 29 nucleotides downstream), or ‘AA’/′CC′, for which transcription initiation occurred at the −1 position (being a ‘C’). The ‘UU’ variant was not in the test panel of 256U1 variants. For the panel of variants, the targeting sequence length was reduced to 13 nucleotides from 15 nucleotides in order to be able to test a number of different dinucleotide variants wherein neither, one or both nucleotides could partake in base pairing with the target sequence, depending on the predicted transcription start site (according to Yeh et al (2017)). It was shown that modified U1 snRNAs harbouring targeting sequences of 9 to 15 nucleotides are all capable of mediating titre increase (FIG. 37).

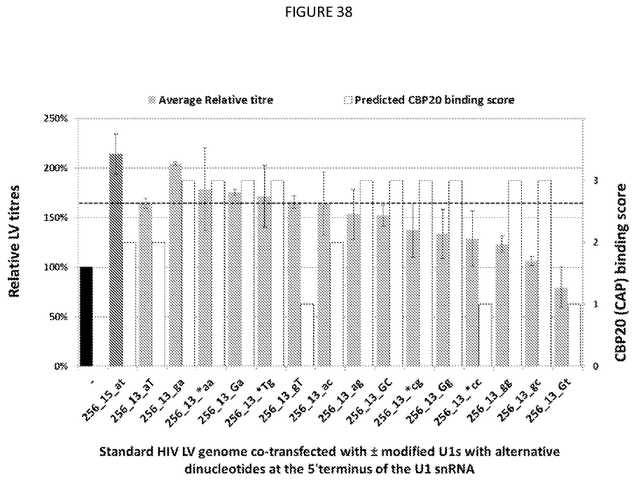

Accordingly, the total length of contiguous targeting sequence was 13 nucleotides for all variants (albeit, not precisely the same 13 nucleotides), and the T-melting temperature for all variants was predicted to be ˜46° C. HIV-EF1a-GFP vector was produced in suspension (serum-free) HEK293T cells at 24-well scale, and each of the dinucleotide variant U1 snRNA co-transfected individually with vector components. Clarified vector harvests were titrated by transduction of adherent HEK293T cells, followed by flow cytometry. The data shown in FIG. 38 displays relative vector titres compared to ‘no 256U1’ indicates that all dinucleotide variants except 256_13_Gt were able to increase vector titres, with most achieving a similar magnitude of enhancement to the control 256_13_aT U1 snRNA. Moreover, there appeared to be no correlation between the predicted CBC binding score of each dinucleotide variant (according to Yeh et al (2017)), and the ability of each variant to mediate a vector titre increase. This indicates that the known CAP-binding properties of U1 snRNA in generating a proficient U1 snRNP splicing complex, are not important for the vector titre enhancement effect of modified U1 snRNAs as described herein. Given that variant 256_13_gT (having the same dinucleotide as 256_13_Gt) was able to mediate a vector titre increase, this also indicated that there may not necessarily be dinucleotides to avoid (other than perhaps rUU′ as mentioned above)—note that the targeting sequences of these two variant U1 snRNAs differed by two nucleotides (one at each end). It was interesting to note that the 256_13_ga variant showed the greatest vector titre enhancement, and so this demonstrated that optimisation of modified U1 snRNA by using different dinucleotides is possible, in addition to screening for the best target sequence within the lentiviral vector packaging region.

### Example 23

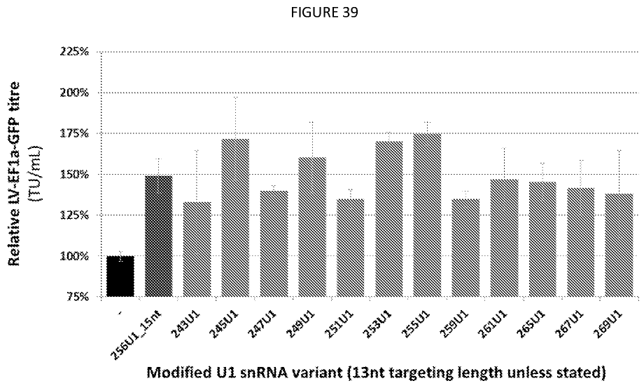

Fine-tuning of target site modification of U1 snRNA by incremental scanning. Other examples herein utilise the 256U1[15nt] modified U1 snRNA variant for increasing titres of HIV-1 based lentiviral vector, as it generally produces the greatest increase in LV titres. To assess if other target sites closer to the 256-270 target region might allow for slight improvements in titre increase, a set of variant modified U1 snRNAs were designed by effectively moving the target sequence ‘window’ up or down from the 256-270 target region by ˜2nt increments (Table VI). All of the variants comprised targeting lengths of 13 nucleotides in order to gain better precision of the importance of slight changes in target sequence in this region (elsewhere it is shown that modified U1 snRNA containing target-annealing lengths of 13 or 15 nucleotides are functionally similar; see FIG. 37). These variant modified U1 snRNAs were individually co-transfected (210 ng/mL plasmid input—see Example 18) with HIV-1 based LV-EF1a-GFP vector components into suspension (serum-free) HEK293T cells, and resulting clarified vector supernatants titrated on adherent HEK293T cells (FIG. 39). The mean average results from two independent experiments show that in this case 253U1-13nt, 255U1-13nt and 245U1-13nt variant modified U1 snRNAs enabled a slightly improved titre enhancement effect compared to the use of 256U1-15nt. The data indicate that even for LV genomes that are modestly boosted by the use of modified U1 snRNA (such as in this case HIV-EF1a-GFP), the precise target sequence may be fine-tuned in order to maximise the titre increase, by altering specific target site and target-annealing length.

TABLE VI
A list of sequences describing the target-annealing sequences (heterologous
sequence that is complementary to the target sequence) within variant modified U1
snRNAs and their target sequences used in the fine-tuning study. Nucleotides are
presented as DNA as they would be encoded within their respective expression
cassettes at the ‘retargeting region’. The (AT) dinucleotide was present in all
constructs, which forms the first two nucleotides of the U1 snRNA molecule in each
case. All variants contained targeting lengths of 13 nucleotides, and target sites
were effectively moved upstream or downstream of the 256U1 targeting site (256U1_15 nt
sequence shown for context). The bold ‘T’ nucleotides in two of the variants partake
in base pairing with the target (maintaining the 13 nucleotide lengths for all
variants). The target sequence numbers refer to targets in the NL4-3 (GenBank:
M19921.2) or HXB2 (GenBank: K03455.1) strains of HIV-1 where denoted, since the
lentiviral vector genome in this study contained a hybrid packaging signal composed
of these two highly conserved strains (packaging sequence was most similar to the
vector sequence in GenBank: MH782475.1).

| Redirected U1 | | U1 snRNA target-annealing |
|---|---|---|
| snRNA* | HIV-1 target sequence [NL4-3] | sequence |
| U1_243 | 243-CAGCAAGCCGAGT-255 | (AT)ACTCGGCTTGCTG |
| | (SEQ ID NO: 93) | (SEQ ID NO: 94) |
| U1_245 | 245-TCGGCTTGCTGAA-257 | (AT)TCAGCAAGCCGA |
| | (SEQ ID NO: 95) | (SEQ ID NO: 96) |
| U1_247 | 247-GGCTTGCTGAAGC-259 | (AT)GCTTCAGCAAGCC |
| | (SEQ ID NO: 97) | (SEQ ID NO: 98) |
| U1_249 | 249-CTTGCTGAAGCGC-261 | (AT)GCGCTTCAGCAAG |
| | (SEQ ID NO: 99) | (SEQ ID NO: 100) |
| U1_251 | 251-TGCTGAAGCGCGC-263 | (AT)GCGCGCTTCAGCA |
| | (SEQ ID NO: 101) | (SEQ ID NO: 102) |
| U1_253 | 253-CTGAAGCGCGCAC-265 | (AT)GTGCGCGCTTCAG |
| | (SEQ ID NO: 103) | (SEQ ID NO: 104) |
| U1_255 | 255-GAAGCGCGCACGG-267 | (AT)CCGTGCGCGCTTC |
| | (SEQ ID NO: 105) | (SEQ ID NO: 106) |
| (U1_256-15 nt) | 256-AAGCGCGCACGGCAA-270 | (AT)TTGCCGTGCGCGCTT |
| | (SEQ ID NO: 107) | (SEQ ID NO: 108) |
| U1_259 | 259-CGCGCACGGCAAG-271 | (AT)CTTGCCGTGCGCG |
| | (SEQ ID NO: 109) | (SEQ ID NO: 110) |
| U1_261 | 261-CGCACGGCAAGAG-273 | (AT)CTCTTGCCGTGCG |
| | (SEQ ID NO: 111) | (SEQ ID NO: 112) |
| U1_263 | 263-CACGGCAAGAGGC-275 | (AT)GCCTCTTGCCGTG |
| | (SEQ ID NO: 113) | (SEQ ID NO: 114) |
| U1_265 | 265-CGGCAAGAGGCGA-277 | (AT)CGCCTCTTGCCG |
| | (SEQ ID NO: 115) | (SEQ ID NO: 116) |
| U1_267 | 267-GCAAGAGGCGAGG-279 | (AT)CCTCGCCTCTTGC |
| | (SEQ ID NO: 117) | (SEQ ID NO: 118) |
| U1_269 | 269-AAGAGGCGAGGGG-281 | (AT)CCCCTCGCCTCTT |
| | (SEQ ID NO: 119) | (SEQ ID NO: 120) |

*numbering relative to vector genome RNA sequence

### Example 24

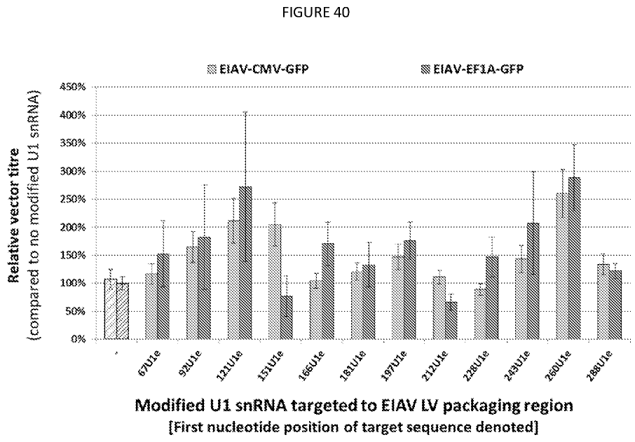

Enhancement of output titres of non-primate lentiviral vectors using modified U1 snRNAs targeted to the 5′ packaging signal sequence region. To assess if modified U1 snRNAs might allow enhancement of non-primate lentiviral vectors, a panel of modified U1 snRNAs with 15nt targeting sequence lengths were designed against the 5′packaging region of EIAV vector genomes (Table VII). These variants targeted from the R region to the retained gag region of the packaging sequence. EIAV-CMV-GFP and EIAV-EF1a-GFP vectors were produced in suspension (serum-free) HEK293T cells with or without the modified eU1 snRNA expression constructs. Clarified vector harvests were titrated by transduction of adherent HEK293T cells, followed by flow cytometry; relative titres were plotted compared to no U1 snRNA (FIG. 40). The data show that the output titres of EIAV-based LVs can be increased (in this case by up to 300%) by use of modified U1 snRNAs, in a similar manner as observed for HIV-1 based LVs, indicating a general mechanism of action. The screen identified optimal target sites on or close to the primer binding site region (121U1e) and within the first nucleotides of the gag sequence (260U1e).

TABLE VII
Modified U1 snRNAs targeted to the 5’packaging region of EIAV-based lentiviral
vector genome vRNA. The table shows the name and target sequence of the modified
U1 snRNA (all 15 nt retargeting sequences) in the EIAV vector genome vRNA (strain
SPEIAV-19). The underlined nucleotide reflects an ATG mutation within the gag
region of the EIAV packaging region.

| Redirected U1 | | U1 snRNA target-annealing |
|---|---|---|
| snRNA* | EIAV target sequence [SPEIAV-19] | sequence |
| eU1_67 | 67-AATTCTCTACTCAGT-81 | (AT)ACTGAGTAGAGAATT |
| | (SEQ ID NO: 121) | (SEQ ID NO: 122) |
| eU1_92 | 92-AGTTTGTCTGTTCGA-106 | (AT)TCGAACAGACAAACT |
| | (SEQ ID NO: 123) | (SEQ ID NO: 124) |
| eU1_121 | 121-CGCCCGAACAGGGAC-135 | (AT)GTCCCTGTTCGGGCG |
| | (SEQ ID NO: 125) | (SEQ ID NO: 126) |
| eU1_151 | 151-ACCCTACCTGTTGAA-165 | (AT)TTCAACAGGTAGGGT |
| | (SEQ ID NO: 127) | (SEQ ID NO: 128) |
| eU1_166 | 166-CCTGGCTGATCGTAG-180 | (AT)CTACGATCAGCCAGG |
| | (SEQ ID NO: 129) | (SEQ ID NO: 130) |
| eU1_181 | 181-GATCCCCGGGACAGC-195 | (AT)GCTGTCCCGGGGATC |
| | (SEQ ID NO: 131) | (SEQ ID NO: 132) |
| eU1_197 | 197-GAGGAGAACTTACAG-211 | (AT)CTGTAAGTTCTCCTC |
| | (SEQ ID NO: 133) | (SEQ ID NO: 134) |
| eU1_212 | 212-AAGTCTTCTGGAGGT-226 | (AT)ACCTCCAGAAGACTT |
| | (SEQ ID NO: 135) | (SEQ ID NO: 136) |
| eU1_228 | 228-TTCCTGGCCAGAACA-242 | (AT)TGTTCTGGCCAGGAA |
| | (SEQ ID NO: 137) | (SEQ ID NO: 138) |
| eU1_243 | 243-CAGGAGGACAGGTAA-257 | (AT)TTACCTGTCCTCCTG |
| | (SEQ ID NO: 139) | (SEQ ID NO: 140) |
| eU1_260 | 260-TTGGGAGACCCTTTG-273 | (AT)CAAAGGGTCTCCCAA |
| | (SEQ ID NO: 141) | (SEQ ID NO: 142) |
| eU1_288 | 286-GCGCTCAAGAAGTTA-300 | (AT)TAACTTCTTGAGCGC |
| | (SEQ ID NO: 143) | (SEQ ID NO: 144) |

*numbering relative to vector genome RNA seguence

### Example 25

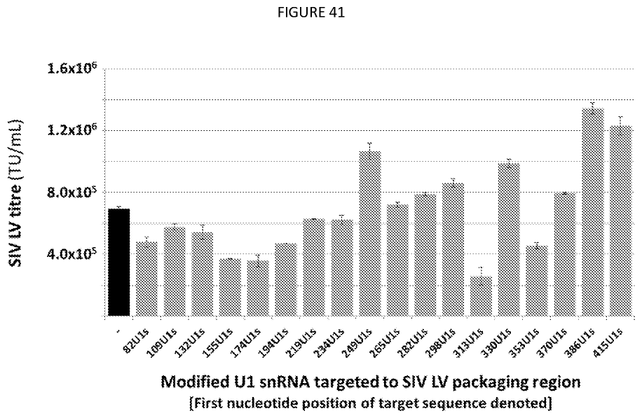

Enhancement of output titres of non-human primate lentiviral vectors (SlVagm) using modified U1 snRNAs targeted to the 5′ packaging signal sequence region. To assess if modified U1 snRNAs might allow enhancement of non-human primate lentiviral vectors, a panel of modified U1 snRNAs with 15nt targeting sequence lengths were designed against the 5′packaging region of SIV vector genomes (Table VIII). These variants targeted from the R region to the retained gag region of the packaging sequence. SIV vectors were produced in suspension (serum-free) HEK293T cells with or without the modified U1 snRNA expression constructs containing target-annealing sequences as outlined in Table VIII. Clarified vector harvests were titrated by transduction of adherent HEK293T cells, followed by integration assay; titres were plotted compared to no U1 snRNA (FIG. 41). The data show that modified U1 snRNA targeted to the 5′ packaging signal sequence region can increase SIV vector titres by 2-fold (see 386U1s and 415U1s). There appeared to be a ‘hotspot’ for targeting the sequence just upstream of the retained gag sequence that constitutes part of the packaging sequence.

TABLE VIII
Modified U1 snRNAs targeted to the 5’packaging region of SIVagm-based
lentiviral vector genome vRNA. The table shows the name and target sequence
of the modified U1 snRNA (all 15 nt retargeting sequences) in the SIVagm
vector genome vRNA (strain TYO-1).

| Redirected U1 | | U1 snRNA target-annealing |
|---|---|---|
| snRNA* | SIVagm target sequence | sequence |
| sU1_82 | 82-CTTGGCTTAGAAAGC-96 | (AT)GCTTTCTAAGCCAAG |
| | (SEQ ID NO: 145) | (SEQ ID NO: 146) |
| sU1_109 | 109-CTGCATTAGAGCTTA-123 | (AT)TAAGCTCTAATGCAG |
| | (SEQ ID NO: 147) | (SEQ ID NO: 148) |
| sU1_132 | 132-AAGTGTCCTCATTGA-146 | (AT)TCAATGAGGACACTT |
| | (SEQ ID NO: 149) | (SEQ ID NO: 150) |
| sU1_155 | 155-TCTCTTGAACGGGAA-169 | (AT)TTCCCGTTCAAGAGA |
| | (SEQ ID NO: 151) | (SEQ ID NO: 152) |
| sU1_174 | 174-CCTTACTGGGTTCTC-188 | (AT)GAGAACCCAGTAAGG |
| | (SEQ ID NO: 153) | (SEQ ID NO: 154) |
| sU1_194 | 194-GACCCAGGCGAGAGA-208 | (AT)TCTCTCGCCTGGGTC |
| | (SEQ ID NO: 155) | (SEQ ID NO: 156) |
| sU1_219 | 219-GTGGCGCCCGAACAG-233 | (AT)CTGTTCGGGCGCCAC |
| | (SEQ ID NO: 157) | (SEQ ID NO: 158) |
| sU1_234 | 234-GGACTTGAGTGAGAG-248 | (AT)CTCTCACTCAAGTCC |
| | (SEQ ID NO: 159) | (SEQ ID NO: 160) |
| sU1_249 | 249-TGTAGGCACGTACAG-263 | (AT)CTGTACGTGCCTACA |
| | (SEQ ID NO: 161) | (SEQ ID NO: 162) |
| sU1_265 | 265-TGAGAAGGCGTCGGA-279 | (AT)TCCGACGCCTTCTCA |
| | (SEQ ID NO: 163) | (SEQ ID NO: 164) |
| sU1_282 | 282-CGAAGGAAGCGCGGG-296 | (AT)CCCGCGCTTCCTTCG |
| | (SEQ ID NO: 165) | (SEQ ID NO: 166) |
| sU1_298 | 298-TGCGACGCGACCAAG-312 | (AT)CTTGGTCGCGTCGCA |
| | (SEQ ID NO: 167) | (SEQ ID NO: 168) |
| sU1_313 | 313-AAGGAGACTTGGTGA-327 | (AT)TCACCAAGTCTCCTT |
| | (SEQ ID NO: 169) | (SEQ ID NO: 170) |
| sU1_330 | 330-AGGCTTCTCGAGTGC-344 | (AT)GCACTCGAGAAGCCT |
| | (SEQ ID NO: 171) | (SEQ ID NO: 172) |
| sU1_352 | 352-AAGCTCGAGCCTAGT-366 | (AT)ACTAGGCTCGAGCTT |
| | (SEQ ID NO: 173) | (SEQ ID NO: 174) |
| sU1_370 | 379-AGGACTAGGAGAGGC-384 | (AT)GCCTCTCCTAGTCCT |
| | (SEQ ID NO: 175) | (SEQ ID NO: 176) |
| sU1_386 | 386-GTAGCCGTAACTACT-400 | (AT)AGTAGTTACGGCTAC |
| | (SEQ ID NO: 177) | (SEQ ID NO: 178) |
| sU1_415 | 415-GCAGGCGGTGGGTAC-429 | (AT)GTACCCACCGCCTGC |
| | (SEQ ID NO: 179) | (SEQ ID NO: 180) |

*numbering relative to vector genome RNA seguence

All publications mentioned in the above specification are herein incorporated by reference. Various modifications and variations of the described methods and system of the invention will be apparent to those skilled in the art without departing from the scope and spirit of the invention.

Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A modified U1 snRNA, wherein said modified U1 snRNA has been modified to bind to a nucleotide sequence within the packaging region of a lentiviral vector genome sequence.

2. The modified U1 snRNA of claim 1, wherein said modified U1 snRNA is modified to introduce a heterologous sequence that is complementary to said nucleotide sequence.

3. The modified U1 snRNA of claim 2, wherein said modified U1 snRNA:
is modified at the 5′ end to introduce within the nine nucleotides at positions 3-to-11 said heterologous sequence;
is modified at the 5′ end to introduce within the native splice donor annealing sequence said heterologous sequence, optionally wherein 1-9 nucleic acids of said native splice donor annealing sequence are replaced with said heterologous sequence; and/or
is modified at the 5′ end to replace a sequence encompassing the native splice donor annealing sequence with a heterologous sequence that is complementary to said nucleotide sequence.

4-6. (canceled)

7. The modified U1 snRNA of claim 2, wherein said heterologous sequence comprises at least 9 nucleotides of complementarity to said nucleotide sequence, optionally wherein said heterologous sequence comprises 15 nucleotides of complementarity to said nucleotide sequence.

8. (canceled)

9. The modified U1 snRNA of claim 1, wherein said packaging region of a lentiviral vector genome sequence is the beginning of the 5′ U5-domain to the terminus of the sequence derived from gag gene.

10. The modified U1 snRNA of claim 1, wherein said nucleotide sequence:
is located within the 5′ U5 domain, PBS element, SL1 element, SL2 element, SL3ψ element, SL4 element and/or the sequence derived from gag gene;
is located within the SL1, SL2 and/or SL3ψ element(s);
is located within the SL1 and/or SL2 element(s); and/or
is located within the SL1 element.

11-15. (canceled)

16. The modified U1 snRNA of claim 1, wherein said lentiviral vector is derived from HIV-1, HIV-2, SIV, FIV, BIV, EIAV, CAEV or Visna lentivirus.

17. (canceled)

18. (canceled)

19. An expression cassette comprising a nucleotide sequence encoding the modified U1 snRNA according to claim 1.

20. A cell for producing lentiviral vectors comprising nucleotide sequences encoding vector components including gag, env, rev and the RNA genome of the lentiviral vector and at least one nucleotide sequence encoding the modified U1 snRNA according to claim 1.

21. A cell comprising the modified U1 snRNA according to claim 1.

22-26. (canceled)

27. A stable or transient production cell for producing lentiviral vectors comprising at least one nucleotide sequence encoding the modified U1 snRNA according to claim 1.

28-30. (canceled)

31. A method for producing a lentiviral vector, comprising the steps of:
a. introducing nucleotide sequences encoding vector components including gag, env, rev and the RNA genome of the lentiviral vector, and at least one nucleotide sequence encoding the modified U1 snRNA according to claim 1, into a cell;
b. optionally selecting for a cell which comprises said nucleotide sequences encoding vector components and at least one modified U1 snRNA;
c. culturing the cell under conditions in which said vector components are co-expressed with said modified U1 snRNA and the lentiviral vector is produced.

32-38. (canceled)

39. The cell according to claim 20, wherein the RNA genome of the lentiviral vector comprises an inactivated major splice donor site.

40. The cell according to claim 39, wherein the RNA genome of the lentiviral vector comprises an inactivated cryptic splice donor site 3′ to the inactivated major splice donor site.

41. (canceled)

42. The cell according to claim 40, wherein;
the lentiviral vector is a tat-independent lentiviral vector;
the lentiviral vector is a U3-independent vector;
the cryptic splice donor site is the first cryptic splice donor site 3′ to the major splice donor site;
the cryptic splice donor site is within 6 nucleotides of the major splice donor site; and/or
the major splice donor site and cryptic splice donor site are mutated or deleted.

43. The cell according to claim 20, wherein the cell does not comprise tat.

44-50. (canceled)

51. The cell, the stable or transient production cell, or the method according to any one of claims 39-50, or the use according to any one of claim 38 or 47-50, wherein the nucleotide sequence encoding the RNA genome of the lentiviral vector prior to inactivation of the splice sites comprises a sequence as set forth in any of SEQ ID NOs: 1, 3, 4, 9, 10 and/or 13.

52. The cell according to claim 40, wherein the nucleotide sequence encoding the RNA genome of the lentiviral vector comprises a sequence with a mutation or deletion relative to the sequence as set forth in any of SEQ ID NOs: 1, 3, 4, 9, 10 and/or 13.

53-56. (canceled)

57. The cell according to claim 39, wherein the nucleotide sequence encoding the RNA genome of the lentiviral vector comprises a sequence as set forth in any of SEQ ID NOs: 2, 5, 6, 7, 8, 11, 12 and/or 14.

58. The cell according to claim 39, wherein the nucleotide sequence encoding the RNA genome of the lentiviral vector does not comprise a sequence as set forth in SEQ ID NO:9.

59. The cell according to claim 39, wherein the splicing activity from the major splice donor site and cryptic splice donor site of the RNA genome of the lentiviral vector is suppressed or ablated.

60. (canceled)

61. The cell according to claim 20, wherein the nucleotide sequence encoding the RNA genome of the lentiviral vector is operably linked to the nucleotide sequence encoding the modified U1 snRNA.

62. (canceled)


SEQUENCE LISTING (192 sequences)

SEQ ID NO: 1 - DNA, 30 bp, Artificial Sequence
  splice donor region
ggggcggcgactggtgagtacgccaaaaat

SEQ ID NO: 2 - DNA, 30 bp, Artificial Sequence
  mutated splice donor region (MSD-2KO)
ggggcggcgactgcagacaacgccaaaaat

SEQ ID NO: 3 - DNA, 8 bp, Artificial Sequence
  major splice donor site consensus sequence
tggtragt

SEQ ID NO: 4 - DNA, 5 bp, Artificial Sequence
  major splice donor consensus sequence
ctggt

SEQ ID NO: 5 - DNA, 6 bp, Artificial Sequence
  splice donor region
cagaca

SEQ ID NO: 6 - DNA, 19 bp, Artificial Sequence
  mutated splice donor region
ggcgactgcagacaacgcc

SEQ ID NO: 7 - DNA, 9 bp, Artificial Sequence
  mutated splice donor region
gtggagact

SEQ ID NO: 8 - DNA, 19 bp, Artificial Sequence
  mutated splice donor region
ggcgagtggagactacgcc

SEQ ID NO: 9 - DNA, 19 bp, Artificial Sequence
  splice donor region
ggcgactggtgagtacgcc

SEQ ID NO: 10 - DNA, 5 bp, Artificial Sequence
  cryptic splice donor site consensus sequence
tgagt

SEQ ID NO: 11 - DNA, 30 bp, Artificial Sequence
  mutated splice donor region (MSD-2KOv2)
ggggcggcgagtggagactacgccaaaaat

SEQ ID NO: 12 - DNA, 32 bp, Artificial Sequence
  mutated splice donor region (MSD-2KOm5)
ggggaaggcaacagataaatatgccttaaaat

SEQ ID NO: 13 - DNA, 7 bp, Artificial Sequence
  major splice donor and cryptic splice donor region core sequence
gtgagta

SEQ ID NO: 14 - DNA, 23 bp, Artificial Sequence
  mutated splice donor region
aaggcaacagataaatatgcctt

SEQ ID NO: 15 - DNA, 642 bp, Artificial Sequence
  example 256U1 (U1_256) snRNA sequence
taaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcccaagatctcatttgccgtgcgcgcttgcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctggtttcaaaagtagactgtacgctaagggtcatatctttttttgttttggtttgtgtcttggttggcgtcttaaatgttaa

SEQ ID NO: 16 - DNA, 15 bp, Artificial Sequence
  HIV-1 target sequence
gaccagatctgagcc

SEQ ID NO: 17 - DNA, 17 bp, Artificial Sequence
  U1 snRNA target-annealing sequence
atggctcagatctggtc

SEQ ID NO: 18 - DNA, 15 bp, Artificial Sequence
  HIV-1 target sequence
tgggagctctctggc

SEQ ID NO: 19 - DNA, 17 bp, Artificial Sequence
  U1 snRNA target-annealing sequence
atgccagagagctccca

SEQ ID NO: 20 - DNA, 15 bp, Artificial Sequence
  HIV-1 target sequence
taaagcttgccttga

SEQ ID NO: 21 - DNA, 17 bp, Artificial Sequence
  U1 snRNA target-annealing sequence
attcaaggcaagcttta

SEQ ID NO: 22 - DNA, 15 bp, Artificial Sequence
  HIV-1 target sequence
tagagatccctcaga

SEQ ID NO: 23 - DNA, 17 bp, Artificial Sequence
  U1 snRNA target-annealing sequence
attctgagggatctcta

SEQ ID NO: 24 - DNA, 9 bp, Artificial Sequence
  HIV-1 target sequence
gcagtggcg

SEQ ID NO: 25 - DNA, 11 bp, Artificial Sequence
  U1 snRNA target-annealing sequence
atcgccactgc

SEQ ID NO: 26 - DNA, 15 bp, Artificial Sequence
  HIV-1 target sequence
agtggcgcccgaaca

SEQ ID NO: 27 - DNA, 17 bp, Artificial Sequence
  U1 snRNA target-annealing sequence
attgttcgggcgccact

SEQ ID NO: 28 - DNA, 15 bp, Artificial Sequence
  HIV-1 target sequence
gggacttgaaagcga

SEQ ID NO: 29 - DNA, 17 bp, Artificial Sequence
  U1 snRNA target-annealing sequence
attcgctttcaagtccc

SEQ ID NO: 30 - DNA, 15 bp, Artificial Sequence
  HIV-1 target sequence
aagggaaaccagagg

SEQ ID NO: 31 - DNA, 17 bp, Artificial Sequence
  U1 snRNA target-annealing sequence
atcctctggtttccctt

SEQ ID NO: 32 - DNA, 15 bp, Artificial Sequence
  HIV-1 target sequence
ggactcggcttgctg

SEQ ID NO: 33 - DNA, 17 bp, Artificial Sequence
  U1 snRNA target-annealing sequence
atcagcaagccgagtcc

SEQ ID NO: 34 - DNA, 15 bp, Artificial Sequence
  HIV-1 target sequence
aagcgcgcacggcaa

SEQ ID NO: 35 - DNA, 17 bp, Artificial Sequence
  U1 snRNA target-annealing sequence
atttgccgtgcgcgctt

SEQ ID NO: 36 - DNA, 15 bp, Artificial Sequence
  HIV-1 target sequence
gaggcgaggggcggc

SEQ ID NO: 37 - DNA, 17 bp, Artificial Sequence
  U1 snRNA target-annealing sequence
atgccgcccctcgcctc

SEQ ID NO: 38 - DNA, 15 bp, Artificial Sequence
  HIV-1 target sequence
gactggtgagtacgc

SEQ ID NO: 39 - DNA, 17 bp, Artificial Sequence
  U1 snRNA target-annealing sequence
atgcgtactcaccagtc

SEQ ID NO: 40 - DNA, 11 bp, Artificial Sequence
  HIV-1 target sequence
aattttgacta

SEQ ID NO: 41 - DNA, 11 bp, Artificial Sequence
  U1 snRNA target-annealing sequence
atgtcaaaatt

SEQ ID NO: 42 - DNA, 15 bp, Artificial Sequence
  HIV-1 target sequence
aattttgactagcgg

SEQ ID NO: 43 - DNA, 17 bp, Artificial Sequence
  U1 snRNA target-annealing sequence
atccgctagtcaaaatt

SEQ ID NO: 44 - DNA, 15 bp, Artificial Sequence
  HIV-1 target sequence
gcggaggctagaagg

SEQ ID NO: 45 - DNA, 17 bp, Artificial Sequence
  U1 snRNA target-annealing sequence
atccttctagcctccgc

SEQ ID NO: 46 - DNA, 15 bp, Artificial Sequence
  HIV-1 target sequence
agagagatgggtgcg

SEQ ID NO: 47 - DNA, 17 bp, Artificial Sequence
  U1 snRNA target-annealing sequence
atcgcacccatctctct

SEQ ID NO: 48 - DNA, 15 bp, Artificial Sequence
  HIV-1 target sequence
agagcgtcggtatta

SEQ ID NO: 49 - DNA, 17 bp, Artificial Sequence
  U1 snRNA target-annealing sequence
attaatactgacgctct

SEQ ID NO: 50 - DNA, 15 bp, Artificial Sequence
  HIV-1 target sequence
agcgggggagaatta

SEQ ID NO: 51 - DNA, 17 bp, Artificial Sequence
  U1 snRNA target-annealing sequence
attaattctcccccgct

SEQ ID NO: 52 - DNA, 15 bp, Artificial Sequence
  HIV-1 target sequence
gatcgcgatgggaaa

SEQ ID NO: 53 - DNA, 17 bp, Artificial Sequence
  U1 snRNA target-annealing sequence
attttcccatcgcgatc

SEQ ID NO: 54 - DNA, 15 bp, Artificial Sequence
  HIV-1 target sequence
aaattcggttaaggc

SEQ ID NO: 55 - DNA, 17 bp, Artificial Sequence
  U1 snRNA target-annealing sequence
atgccttaaccgaattt

SEQ ID NO: 56 - DNA, 15 bp, Artificial Sequence
  HIV-1 target sequence
gatcttcagacctgg

SEQ ID NO: 57 - DNA, 17 bp, Artificial Sequence
  U1 snRNA target-annealing sequence
atccaggtctgaagatc

SEQ ID NO: 58 - DNA, 15 bp, Artificial Sequence
  HIV-1 target sequence
ttacacaagcttaat

SEQ ID NO: 59 - DNA, 17 bp, Artificial Sequence
  U1 snRNA target-annealing sequence
atattaagcttgtgtaa

SEQ ID NO: 60 - DNA, 15 bp, Artificial Sequence
  HIV-1 target sequence
tagtagacataatag

SEQ ID NO: 61 - DNA, 17 bp, Artificial Sequence
  U1 snRNA target-annealing sequence
atctattatgtctacta

SEQ ID NO: 62 - DNA, 9 bp, Artificial Sequence
  Control U1 snRNA target sequence
ctacaggaa

SEQ ID NO: 63 - DNA, 11 bp, Artificial Sequence
  U1 snRNA target-annealing sequence
atttcctgtag

SEQ ID NO: 64 - DNA, 9 bp, Artificial Sequence
  Control U1 snRNA target sequence
tcatctgtg

SEQ ID NO: 65 - DNA, 11 bp, Artificial Sequence
  U1 snRNA target-annealing sequence
atcacagatga

SEQ ID NO: 66 - DNA, 153 bp, Artificial Sequence
  modified U1 snRNA sequence (main U1 snRNA sequence [clover leaf] (nt 410-562) of the U1_256 sequence)
gcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctg

SEQ ID NO: 67 - DNA, 673 bp, Human immunodeficiency virus type 1
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaaga

SEQ ID NO: 68 - DNA, 996 bp, Human immunodeficiency virus type 2
cagtcgctctgcggagaggctggcagatcgagccctgagaggttctctccagcactagcaggtagagcctgggtgttccctgctggactctcaccagtacttggccggtactgggcagacggctccacgcttgcttgcttaaagacctcttcaataaagctgccagttagaagcaagttaagtgtgtgttcccatctctcctagtcgccgcctggtcattcggtgttcatctgagtaacaagaccctggtctgttaggacccttctcgctttgggaatccaaggcaggaaaatccctagcaggttggcgcccgaacagggacttgaagaggactgagaagccctggaactcggctgagtgaaggcagtaagggcggcaggaacaaaccacgacggagtgctcctagaaaggcgcgggccgaggtaccaaaggcggcgtgtggagcgggagtgaaagaggcctccgggtgaaggtaagtacctacaccaaaaactgtagccagaaaaggcttgttatcctacctttagacaggtagaagattgtgggagatgggcgcgagaaactccgtcttgagagggaaaaaagcagacgaattagaaaaagttaggttacggcccggcggaaagaaaaagtacaggttaaaacatattgtgtgggcagcgaatgaattggataaattcggattggcagagagcctgttggagtcaaaagaaggttgccaaaagattctcagagttttagatccattagtaccaacagggtcagaaaatttaaaaagcctttttaataccgtctgcgtcatttggtgcttgcacgcagaagagaaagtgaaagatactgaggaagcaaagaaactagcacagagacatctagtggcagaaactggaactgcagagaaaatgccaaatacaagtagaccaacagcaccacctagtgggaaaagaggaaactaccccgtgcaacaagcgggtggcaactatgtccatgtgccactga

SEQ ID NO: 69 - DNA, 630 bp, Equine infectious anemia virus
gggactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagatgggagaccctttgacatggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatc

SEQ ID NO: 70 - DNA, 781 bp, Simian immunodeficiency virus
cagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtactatattgcttgcacaaggaacagaaagtgaaagacacagaggaagcagtagcaacagtaagacaacactgccatctagtggaaaaagaaaaaagtgc

SEQ ID NO: 71 - DNA, 865 bp, Simian immunodeficiency virus
cagtcgctctgcggagaggctggcagattgagccctgggaggttctctccagcactagcaggtagagcctgggtgttccctgctagactctcaccagcacttggccagtgctgggcagagtggctccacgcttgcttgcttaaagacctcttcaataaagctgccattttagaagtaagccagtgtgtgttcccatctctcctagtcgccgcctggtcaactcggtactcggtaataagaagaccctggtctgttaggaccctttctgctttgagaaaccgaagcaggaaaatccctagcagattggcgcccgaacaggacttgaaggagagtgagagactcctgagtacggctgagtgaaggcagtaagggcggcaggaaccaaccacgacggagtgctcctataaaggcgcgggtcggtaccagacggcgtgaggagcgggagaggaggaggcctccggttgcaggtaagtgcaacacaaaaaagaaatagctgtcttgttatccaggaagggataataagatagagtgggagatgggcgcgagaaactccgtcttgtcagggaagaaagcagatgaattagaaaaaattaggctacgacccggcggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtg

SEQ ID NO: 72 - DNA, 764 bp, Feline immunodeficiency virus
gagtctctttgttgaggacttttgagttctcccttgaggctcccacagatacaataaatatttgagattgaaccctgtcgagtatctgtgtaatcttttttacctgtgaggtctcggaatccgggccgagaacttcgcagttggcgcccgaacagggacttgattgagagtgattgaggaagtgaagctagagcaatagaaagctgttaagcagaactcctgctgacctaaatagggaagcagtagcagacgctgctaacagtgagtatctctagtgaagcggactcgagctcataatcaagtcattgtttaaaggcccagataaattacatctggtgactcttcgcggaccttcaagccaggagattcgccgagggacagtcaacaaggtaggagagattctacagcaacatggggaatggacaggggcgagattggaaaatggccattaagagatgtagtaatgttgctgtaggagtaggggggaagagtaaaaaatttggagaagggaatttcagatgggccattagaatggctaatgtatctacaggacgagaacctggtgatataccagagactttagatcaactaaggttggttatttgcgatttacaagaaagaagagaaaaatttggatctagcaaagaaattgatatggcaattgtgacattaaaagtctttgcggtagcaggacttttaaatatgacggtgtctactgctgctgcagctgaaaatatgtattctcaaatgggattagacactag

SEQ ID NO: 73 - DNA, 15 bp, Artificial Sequence
  HIV-1 target sequence
agctctctcgacgca

SEQ ID NO: 74 - DNA, 17 bp, Artificial Sequence
  U1 snRNA target-annealing sequence
attgcgtcgagagagct

SEQ ID NO: 75 - DNA, 21 bp, Artificial Sequence
  target in LV genome
ctgaagcgcgcacggcaagag

SEQ ID NO: 76 - DNA, 17 bp, Artificial Sequence
  variant 256U1 target sequence
atttgccgtgcgcgctt

SEQ ID NO: 77 - DNA, 14 bp, Artificial Sequence
  variant 256U1 target sequence
atgccgtgcgcgct

SEQ ID NO: 78 - DNA, 15 bp, Artificial Sequence
  variant 256U1 target sequence
aggccgtgcgcgctt

SEQ ID NO: 79 - DNA, 16 bp, Artificial Sequence
  variant 256U1 target sequence
caagccgtgcgcgctt

SEQ ID NO: 80 - DNA, 15 bp, Artificial Sequence
  variant 256U1 target sequence
acgccgtgcgcgctt

SEQ ID NO: 81 - DNA, 15 bp, Artificial Sequence
  variant 256U1 target sequence
gggccgtgcgcgctt

SEQ ID NO: 82 - DNA, 15 bp, Artificial Sequence
  variant 256U1 target sequence
gagccgtgcgcgctt

SEQ ID NO: 83 - DNA, 14 bp, Artificial Sequence
  variant 256U1 target sequence
gtgccgtgcgcgct

SEQ ID NO: 84 - DNA, 15 bp, Artificial Sequence
  variant 256U1 target sequence
gcgccgtgcgcgctt

SEQ ID NO: 85 - DNA, 15 bp, Artificial Sequence
  variant 256U1 target sequence
tggccgtgcgcgctt

SEQ ID NO: 86 - DNA, 15 bp, Artificial Sequence
  variant 256U1 target sequence
cggccgtgcgcgctt

SEQ ID NO: 87 - DNA, 16 bp, Artificial Sequence
  variant 256U1 target sequence
cccgccgtgcgcgctt

SEQ ID NO: 88 - DNA, 13 bp, Artificial Sequence
  variant 256U1 target sequence
gccgtgcgcgctt

SEQ ID NO: 89 - DNA, 14 bp, Artificial Sequence
  variant 256U1 target sequence
gacgtgcgcgcttc

SEQ ID NO: 90 - DNA, 14 bp, Artificial Sequence
  variant 256U1 target sequence
gtcgtgcgcgcttc

SEQ ID NO: 91 - DNA, 14 bp, Artificial Sequence
  variant 256U1 target sequence
ggcgtgcgcgcttc

SEQ ID NO: 92 - DNA, 22 bp, Artificial Sequence
  U1 promoter transcription start site
  misc_feature (8)..(22): n is a, c, g, or t
atctcatnnnnnnnnnnnnnnn

SEQ ID NO: 93 - DNA, 13 bp, Artificial Sequence
  HIV-1 target sequence
cagcaagccgagt

SEQ ID NO: 94 - DNA, 15 bp, Artificial Sequence
  U1 snRNA target-annealing sequence
atactcggcttgctg

SEQ ID NO: 95 - DNA, 13 bp, Artificial Sequence
  HIV-1 target sequence
tcggcttgctgaa

SEQ ID NO: 96 - DNA, 14 bp, Artificial Sequence
  U1 snRNA target-annealing sequence
attcagcaagccga

SEQ ID NO: 97 - DNA, 13 bp, Artificial Sequence
  HIV-1 target sequence
ggcttgctgaagc

SEQ ID NO: 98 - DNA, 15 bp, Artificial Sequence
  U1 snRNA target-annealing sequence
atgcttcagcaagcc

SEQ ID NO: 99 - DNA, 13 bp, Artificial Sequence
  HIV-1 target sequence
cttgctgaagcgc

SEQ ID NO: 100 - DNA, 15 bp, Artificial Sequence
  U1 snRNA target-annealing sequence
atgcgcttcagcaag

SEQ ID NO: 101 - DNA, 13 bp, Artificial Sequence
  HIV-1 target sequence
tgctgaagcgcgc

SEQ ID NO: 102 - DNA, 15 bp, Artificial Sequence
  U1 snRNA target-annealing sequence
atgcgcgcttcagca

SEQ ID NO: 103 - DNA, 13 bp, Artificial Sequence
  HIV-1 target sequence
ctgaagcgcgcac

SEQ ID NO: 104 - DNA, 15 bp, Artificial Sequence
  U1 snRNA target-annealing sequence
atgtgcgcgcttcag

SEQ ID NO: 105 - DNA, 13 bp, Artificial Sequence
  HIV-1 target sequence
gaagcgcgcacgg

SEQ ID NO: 106 - DNA, 15 bp, Artificial Sequence
  U1 snRNA target-annealing sequence
atccgtgcgcgcttc

SEQ ID NO: 107 - DNA, 15 bp, Artificial Sequence
  HIV-1 target sequence
aagcgcgcacggcaa

SEQ ID NO: 108 - DNA, 17 bp, Artificial Sequence
  U1 snRNA target-annealing sequence
atttgccgtgcgcgctt

SEQ ID NO: 109 - DNA, 13 bp, Artificial Sequence
  HIV-1 target sequence
cgcgcacggcaag

SEQ ID NO: 110 - DNA, 15 bp, Artificial Sequence
  U1 snRNA target-annealing sequence
atcttgccgtgcgcg

SEQ ID NO: 111 - DNA, 13 bp, Artificial Sequence
  HIV-1 target sequence
cgcacggcaagag

SEQ ID NO: 112 - DNA, 15 bp, Artificial Sequence
  U1 snRNA target-annealing sequence
atctcttgccgtgcg

SEQ ID NO: 113 - DNA, 13 bp, Artificial Sequence
  HIV-1 target sequence
cacggcaagaggc

SEQ ID NO: 114 - DNA, 15 bp, Artificial Sequence
  U1 snRNA target-annealing sequence
atgcctcttgccgtg

SEQ ID NO: 115 - DNA, 13 bp, Artificial Sequence
  HIV-1 target sequence
cggcaagaggcga

SEQ ID NO: 116 - DNA, 14 bp, Artificial Sequence
  U1 snRNA target-annealing sequence
atcgcctcttgccg

SEQ ID NO: 117 - DNA, 13 bp, Artificial Sequence
  HIV-1 target sequence
gcaagaggcgagg

SEQ ID NO: 118 - DNA, 15 bp, Artificial Sequence
  U1 snRNA target-annealing sequence
atcctcgcctcttgc

SEQ ID NO: 119 - DNA, 13 bp, Artificial Sequence
  HIV-1 target sequence
aagaggcgagggg

SEQ ID NO: 120 - DNA, 15 bp, Artificial Sequence
  U1 snRNA target-annealing sequence
atcccctcgcctctt

SEQ ID NO: 121 - DNA, 15 bp, Artificial Sequence
  EIAV target sequence
aattctctactcagt

SEQ ID NO: 122 - DNA, 17 bp, Artificial Sequence
  U1 snRNA target-annealing sequence
atactgagtagagaatt

SEQ ID NO: 123 - DNA, 15 bp, Artificial Sequence
  EIAV target sequence
agtttgtctgttcga

SEQ ID NO: 124 - DNA, 17 bp, Artificial Sequence
  U1 snRNA target-annealing sequence
attcgaacagacaaact

SEQ ID NO: 125 - DNA, 15 bp, Artificial Sequence
  EIAV target sequence
cgcccgaacagggac

SEQ ID NO: 126 - DNA, 17 bp, Artificial Sequence
  U1 snRNA target-annealing sequence
atgtccctgttcgggcg

SEQ ID NO: 127 - DNA, 15 bp, Artificial Sequence
  EIAV target sequence
accctacctgttgaa

SEQ ID NO: 128 - DNA, 17 bp, Artificial Sequence
  U1 snRNA target-annealing sequence
atttcaacaggtagggt

SEQ ID NO: 129 - DNA, 15 bp, Artificial Sequence
  EIAV target sequence
cctggctgatcgtag

SEQ ID NO: 130 - DNA, 17 bp, Artificial Sequence
  U1 snRNA target-annealing sequence
atctacgatcagccagg

SEQ ID NO: 131 - DNA, 15 bp, Artificial Sequence
  EIAV target sequence
gatccccgggacagc

SEQ ID NO: 132 - DNA, 17 bp, Artificial Sequence
  U1 snRNA target-annealing sequence
atgctgtcccggggatc

SEQ ID NO: 133 - DNA, 15 bp, Artificial Sequence
  EIAV target sequence
gaggagaacttacag

SEQ ID NO: 134 - DNA, 17 bp, Artificial Sequence
  U1 snRNA target-annealing sequence
atctgtaagttctcctc

SEQ ID NO: 135 - DNA, 15 bp, Artificial Sequence
  EIAV target sequence
aagtcttctggaggt

SEQ ID NO: 136 - DNA, 17 bp, Artificial Sequence
  U1 snRNA target-annealing sequence
atacctccagaagactt

SEQ ID NO: 137 - DNA, 15 bp, Artificial Sequence
  EIAV target sequence
ttcctggccagaaca

SEQ ID NO: 138 - DNA, 17 bp, Artificial Sequence
  U1 snRNA target-annealing sequence
attgttctggccaggaa

SEQ ID NO: 139 - DNA, 15 bp, Artificial Sequence
  EIAV target sequence
caggaggacaggtaa

SEQ ID NO: 140 - DNA, 17 bp, Artificial Sequence
  U1 snRNA target-annealing sequence
atttacctgtcctcctg

SEQ ID NO: 141 - DNA, 15 bp, Artificial Sequence
  EIAV target sequence
ttgggagaccctttg

SEQ ID NO: 142 - DNA, 17 bp, Artificial Sequence
  U1 snRNA target-annealing sequence
atcaaagggtctcccaa

SEQ ID NO: 143 - DNA, 15 bp, Artificial Sequence
  EIAV target sequence
gcgctcaagaagtta

SEQ ID NO: 144 - DNA, 17 bp, Artificial Sequence
  U1 snRNA target-annealing sequence
attaacttcttgagcgc

SEQ ID NO: 145 - DNA, 15 bp, Artificial Sequence
  SIVagm target sequence
cttggcttagaaagc

SEQ ID NO: 146 - DNA, 17 bp, Artificial Sequence
  U1 snRNA target-annealing sequence
atgctttctaagccaag

SEQ ID NO: 147 - DNA, 15 bp, Artificial Sequence
  SIVagm target sequence
ctgcattagagctta

SEQ ID NO: 148 - DNA, 17 bp, Artificial Sequence
  U1 snRNA target-annealing sequence
attaagctctaatgcag

SEQ ID NO: 149 - DNA, 15 bp, Artificial Sequence
  SIVagm target sequence
aagtgtcctcattga

SEQ ID NO: 150 - DNA, 17 bp, Artificial Sequence
  U1 snRNA target-annealing sequence
attcaatgaggacactt

SEQ ID NO: 151 - DNA, 15 bp, Artificial Sequence
  SIVagm target sequence
tctcttgaacgggaa

SEQ ID NO: 152 - DNA, 17 bp, Artificial Sequence
  U1 snRNA target-annealing sequence
atttcccgttcaagaga

SEQ ID NO: 153 - DNA, 15 bp, Artificial Sequence
  SIVagm target sequence
ccttactgggttctc

SEQ ID NO: 154 - DNA, 17 bp, Artificial Sequence
  U1 snRNA target-annealing sequence
atgagaacccagtaagg

SEQ ID NO: 155 - DNA, 15 bp, Artificial Sequence
  SIVagm target sequence
gacccaggcgagaga

SEQ ID NO: 156 - DNA, 17 bp, Artificial Sequence
  U1 snRNA target-annealing sequence
attctctcgcctgggtc

SEQ ID NO: 157 - DNA, 15 bp, Artificial Sequence
  SIVagm target sequence
gtggcgcccgaacag

SEQ ID NO: 158 - DNA, 17 bp, Artificial Sequence
  U1 snRNA target-annealing sequence
atctgttcgggcgccac

SEQ ID NO: 159 - DNA, 15 bp, Artificial Sequence
  SIVagm target sequence
ggacttgagtgagag

SEQ ID NO: 160 - DNA, 17 bp, Artificial Sequence
  U1 snRNA target-annealing sequence
atctctcactcaagtcc

SEQ ID NO: 161 - DNA, 15 bp, Artificial Sequence
  SIVagm target sequence
tgtaggcacgtacag

SEQ ID NO: 162 - DNA, 17 bp, Artificial Sequence
  U1 snRNA target-annealing sequence
atctgtacgtgcctaca

SEQ ID NO: 163 - DNA, 15 bp, Artificial Sequence
  SIVagm target sequence
tgagaaggcgtcgga

SEQ ID NO: 164 - DNA, 17 bp, Artificial Sequence
  U1 snRNA target-annealing sequence
attccgacgccttctca

SEQ ID NO: 165 - DNA, 15 bp, Artificial Sequence
  SIVagm target sequence
cgaaggaagcgcggg

SEQ ID NO: 166 - DNA, 17 bp, Artificial Sequence
  U1 snRNA target-annealing sequence
atcccgcgcttccttcg

SEQ ID NO: 167 - DNA, 15 bp, Artificial Sequence
  SIVagm target sequence
tgcgacgcgaccaag

SEQ ID NO: 168 - DNA, 17 bp, Artificial Sequence
  U1 snRNA target-annealing sequence
atcttggtcgcgtcgca

SEQ ID NO: 169 - DNA, 15 bp, Artificial Sequence
  SIVagm target sequence
aaggagacttggtga

SEQ ID NO: 170 - DNA, 17 bp, Artificial Sequence
  U1 snRNA target-annealing sequence
attcaccaagtctcctt

SEQ ID NO: 171 - DNA, 15 bp, Artificial Sequence
  SIVagm target sequence
aggcttctcgagtgc

SEQ ID NO: 172 - DNA, 17 bp, Artificial Sequence
  U1 snRNA target-annealing sequence
atgcactcgagaagcct

SEQ ID NO: 173 - DNA, 15 bp, Artificial Sequence
  SIVagm target sequence
aagctcgagcctagt

SEQ ID NO: 174 - DNA, 17 bp, Artificial Sequence
  U1 snRNA target-annealing sequence
atactaggctcgagctt

SEQ ID NO: 175 - DNA, 15 bp, Artificial Sequence
  SIVagm target sequence
aggactaggagaggc

SEQ ID NO: 176 - DNA, 17 bp, Artificial Sequence
  U1 snRNA target-annealing sequence
atgcctctcctagtcct

SEQ ID NO: 177 - DNA, 15 bp, Artificial Sequence
  SIVagm target sequence
gtagccgtaactact

SEQ ID NO: 178 - DNA, 17 bp, Artificial Sequence
  U1 snRNA target-annealing sequence
atagtagttacggctac

SEQ ID NO: 179 - DNA, 15 bp, Artificial Sequence
  SIVagm target sequence
gcaggcggtgggtac

SEQ ID NO: 180 - DNA, 17 bp, Artificial Sequence
  U1 snRNA target-annealing sequence
atgtacccaccgcctgc

SEQ ID NO: 181 - RNA, 164 nt, Artificial Sequence
  U1 snRNA molecule
auacuuaccuggcaggggagauaccaugaucacgaaggugguuuucccagggcgaggcuuauccauugcacuccggaugugcugaccccugcgauuuccccaaaugugggaaacucuacugcauaauuugugguagugggggacugcguucgcgcuuuccccug

SEQ ID NO: 182 - RNA, 12 nt, Artificial Sequence
  70K WT sequence
ugaucacgaagu

SEQ ID NO: 183 - RNA, 12 nt, Artificial Sequence
  70K m1 sequence
uugacacgaauu

SEQ ID NO: 184 - RNA, 12 nt, Artificial Sequence
  70K m2 sequence
acuagugcuucc

SEQ ID NO: 185 - RNA, 10 nt, Artificial Sequence
  Sm WT sequence
auaauuugug

SEQ ID NO: 186 - RNA, 172 nt, Artificial Sequence
  modified U1A5 snRNA variant sequence
auggccaguuggcagagugaucagaacacacauaacauuuauugaugaaguuugcuaccuugggcuuauccauugcacucuggaugugcugaccccugcaauuuuccacaaaugugagaaacuugacugcauaauuuaugguagugggaggcugcguuugcacucuccccug

SEQ ID NO: 187 - RNA, 166 nt, Artificial Sequence
  modified U1A6 snRNA variant sequence
augucuucguggcgggggagagacgcuguugguccugauaaaguguuuuucuugggggaggguuuuuacuuuguguuugggauguguuugcucccgcgauuuccccgaaugagaaaacucggcugcauaacuugugguagugggggacugcuuuugcgcuuuucug

SEQ ID NO: 188 - RNA, 165 nt, Artificial Sequence
  modified U1A7 snRNA variant sequence
auacuuaacuggcaagggaaauaacaugaucauuaaagguggauuuuucagggugagguuugucucuugcauuguggaugugcugaccccugugguuuucuacaaaugugggaaacuuaauugcauaauuugugguaguggggacuauguugguucucuccccug

SEQ ID NO: 189 - DNA, 13 bp, Artificial Sequence
  stem loop 2 sequence mutation, deltaSL2
ggggcgcaaaaat

SEQ ID NO: 190 - RNA, 170 nt, Artificial Sequence
  256U1 snRNA molecule sequence
auuugccgugcgcgcuucuaggggagauaccaugaucacgaaggugguuuucccagggcgaggcuuauccauugcacuccggaugugcugaccccugcgauuuccccaaaugugggaaacucgacugcauaauuugugguagugggggacugcguucgcgcuuuccccug

SEQ ID NO: 191 - protein, 124 aa, Mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTSVTVSS

SEQ ID NO: 192 - protein, 114 aa, Homo sapiens
ELVLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRRS